(19) United States
(12) Patent Application Publication
(10) Pub. No.: US 2026/0356929 A1
(43) Pub. Date: Nov. 12, 2026

(54) VEGF ANTIBODIES

(71) Applicant: THE UNIVERSITY OF NOTTINGHAM, Nottingham (GB)

(72) Inventor: David BATES, Nottingham (GB)

(21) Appl. No.: 19/119,274

(22) PCT Filed: Oct. 13, 2023
(86) PCT No.: PCT/GB2023/052666

§ 371 Date: Apr. 8, 2025

(30) Foreign Application Priority Data
Oct. 13, 2022 (GB) 2215115.3

(51) Int. Cl. C07K 16/22; A61K 39/00; A61K 47/60; A61K 47/68; A61P 9/10; G01N 33/68

(52) U.S. Cl. CPC C07K 16/22; A61K 47/60; A61K 47/6845; A61P 9/10; G01N 33/6854; A61K 2039/505; C07K 2317/24; C07K 2317/76; C07K 2317/94

(57) ABSTRACT

The present invention relates to anti-VEGF-A165b antibodies. The invention also relates to pharmaceutical compositions, kits and methods of use in therapy.

### FIELD OF THE INVENTION

The present invention relates to targeting of VEGF-A₁₆₅b and monoclonal antibodies, such as monoclonal antibodies (mAbs), which bind VEGF-A₁₆₅b.

### BACKGROUND

Peripheral vascular disease is the leading cause of amputations, and a highly prevalent contributor to cardiovascular deaths. Lack of neovascularisation has been implicated in obese and diabetic patients due to heightened monocyte derived Wnt5a signalling to increase the anti-angiogenic forms of VEGF (Kikuchi et al 2014, Nat Med).

Growth of new blood vessels (angiogenesis), required for all tumour growth, is stimulated by the expression of vascular endothelial growth factor (VEGF). VEGF is upregulated in all known solid tumours but also in atherosclerosis, diabetic retinopathy, arthritis, and many other conditions. Conventional VEGF isoforms have been widely described as pro-angiogenic cytokines. A splice variant of VEGF-A called VEGF-A₁₆₅b is expressed as a protein in normal cells and tissues and circulates in human plasma (see FIG. 34). VEGF-A₁₆₅b binds VEGF receptor 2 with the same affinity as VEGF-A₁₆₅a but does not activate it or stimulate the same downstream signalling pathways. Moreover, it impairs VEGF-A₁₆₅a-mediated VEGF receptor 2 phosphorylation and signalling in cultured cells. VEGF-A₁₆₅b is not angiogenic and it inhibits VEGF-A₁₆₅a-mediated angiogenesis in rabbit cornea, rat mesentery, mouse mammary glands, dorsal chambers and ovaries, as well as in proliferative oxygen-induced retinopathy, choroidal neovascularisation, and cancer growth. VEGF-A₁₆₅a expressing tumours grow significantly more slowly than VEGF-A₁₆₅a-expressing tumours, indicating that a switch in splice variant expression from VEGF-A₁₆₅a to VEGF-A₁₆₅b can inhibit tumour growth.

It has also been previously found that an anti-VEGF-A₁₆₅b mouse monoclonal antibody stimulates revascularisation in a model of hindlimb ischemia in mice who are genetically obese (ob/ob) or who have been fed a high fat high sucrose diet for 12 weeks (Kikuchi et al 2014. Nat Med.; 20 (12): 1464-1471, which is incorporated herein by reference), and that an anti-VEGF-A₁₆₅b mouse monoclonal antibody stimulates revascularisation in a model of impaired hindlimb ischemia in mice who are genetically eNOS deficient, or who overexpress myoglobin (Kuppuswamy et al., Cells 2022, 11, 2676). However, there is a need for therapeutically effective antibodies preferably effective for use in humans.

### SUMMARY OF THE INVENTION

An aim of the present invention is to provide a therapeutic monoclonal antibody against VEGF-A₁₆₅b.

A first aspect of the invention relates to an antibody or antigen-binding fragment thereof with specificity for a splice variant of vascular endothelial growth factor (VEGF), wherein the splice variant is VEGF-A₁₆₅b or any VEGF sequence containing the sequence encoded by Exon 8b of the VEGF gene (such as VEGF-Ax and VEGF₁₈₉b).

In some embodiments, the antibody is an intact antibody. In some embodiments, the antigen-binding fragment is selected from the group consisting of Fv fragments and Fab-like fragments (e.g. Fab fragments, Fab′ fragments and F(ab)₂ fragments). By “Fv fragments”, we include single chain FV, disulphide-bonded Fv and domain antibodies. By “Fab-like fragments”, we include Fab fragments, Fab′ fragments and F(ab)₂ fragments.

In some embodiments, the antibody or antigen-binding fragment thereof is recombinant. In some embodiments, the antibody or antigen-binding fragment thereof is monoclonal. In some embodiments, the antibody or antigen-binding fragment thereof is polyclonal. In some embodiments, the antibody or antigen-binding fragment thereof is murine. In some embodiments, the antibody or antigen-binding fragment thereof is chimeric. In some embodiments, the antibody or antigen-binding fragment thereof is human or humanised.

In some embodiments, the antibody or antigen-binding fragment thereof comprises or consists of the following Complementarity determining regions (CDRs), as determined by the IMGT determination method:

a. V_{H} CDR1:
(SEQ ID NO: 1)
GFDFSRYW;
b. V_{H} CDR2:
(SEQ ID NO: 2)
IHPYSSTI;
c. V_{H} CDR3:
(SEQ ID NO: 3)
ARAFAY;
d. V_{L} CDR1:
(SEQ ID NO: 4)
QSLLDSDGKTY;
e. V_{L} CDR2:
(SEQ ID NO: 5)
LVS;
and/or
f. V_{L} CDR3:
(SEQ ID NO: 6)
WQGTHFPYT.

In some embodiments, the antibody or antigen-binding fragment thereof comprises or consists of the following CDRs, as determined by the Kabat determination method:

a. V_{H} CDR1:
(SEQ ID NO: 7)
RYWMSW;
b. V_{H} CDR2:
(SEQ ID NO: 8)
EIHPYSSTINYTPSVKD;
c. V_{H} CDR3:
(SEQ ID NO: 9)
AFAY;
d. V_{L} CDR1:
(SEQ ID NO: 10)
RSSQSLLDSDGKTYLN;
or
(SEQ ID NO: 11)
KSSQSLLDSDGKTYLN;
e. V_{L} CDR2:
(SEQ ID NO: 12)
LVSKLDS;
and/or
f. V_{L} CDR3:
(SEQ ID NO: 13)
WQGTHFPYT

In some embodiments, the VL CDR1, as determined by the Kabat determination method, is *SSQSLLDSDGKTYLN (SEQ ID NO: 14), wherein * is R or K.

In some embodiments, the CDR sequences are determined by the Kabat determination method. In some embodiments, the CDR sequences are determined by the IMGT determination method.

In some embodiments, the antibody or antigen-binding fragment thereof comprises or consists of the following variable heavy chains:

a.
(SEQ ID NO: 15; also referred to herein as the V_{H} chain
of the parental mouse mAb clone “58/8/31/1/13”)
EVKLLESGGGLVQPGGSLKLSCAASGEDESRYWMSWVRQAPGKGLEWIGEIHPYSSTINYTPSVKDKFI
ISRDNAKNTLYLQMSEVRSEDTALYYCARAFAYWGQGTLVTVSA;
b.
(SEQ ID NO: 15); also referred to herein as “VH0”)
EVKLLESGGGLVQPGGSLKLSCAASGFDFSRYWMSWVRQAPGKGLEWIGEIHPYSSTINYTPSVKDKFI
ISRDNAKNTLYLQMSEVRSEDTALYYCARAFAYWGQGTLVTVSA;
c.
(SEQ ID NO: 16; also referred to herein as “VH1”)
EVQLLESGGGLVKPGGSLRLSCAASGEDESRYWMSWIRQAPGKGLEWVSEIHPYSSTINYTPSVKDRFT
ISRDNAKNSLYLQMNSLRAEDTAVYYCARAFAYWGQGTLVTVSS;
d.
(SEQ ID NO: 17; also referred to herein as “VH2”)
EVQLVESGGGLVQPGGSLRLSCAASGFDFSRYWMSWVRQAPGKGLEWVAEIHPYSSTINYTPSVKDRFI
ISRDNAKNSVYLQLNSLRAEDTAVYYCARAFAYWGQGTLVTVSS;
e.
(SEQ ID NO: 18; also referred to herein as “VH3”)
EVQLVESGGGLVQPGGSLRLSCAASGEDESRYWMSWVRQAPGKGLEWISEIHPYSSTINYTPSVKDRET
ISRDNAKNSLYLQMNSLRDEDTALYYCARAFAYWGQGTLVTVSS;
f.
(SEQ ID NO: 19; also referred to herein as “VH4”)
EVQLVESGGGLVQPGGSLRLSCAASGEDESRYWMSWVRQAPGKGLEWVAEIHPYSSTINYTPSVKDRET
ISRDNAKNSLYLQMNSLRSEDTAVYYCARAFAYWGQGTLVTVSS;
or
g.
(SEQ ID NO: 20; also referred to herein as “VH5”)
EVQLVESGGGLVQPGGSLRLSCAASGEDESRYWMSWVRQAPGKGLEWVSEIHPYSSTINYTPSVKDRET
ISRDNAKNSLYLQMNSLRAEDTAVYYCARAFAYWGQGTLVTVSA.

In some embodiments, the antibody or antigen-binding fragment thereof comprises or consists of the following consensus variable heavy chain sequence, wherein substitutions or deletions in particular positions are indicated in parenthesis, deletions being marked as ‘X’;

(SEQ ID NO: 21)
EVKLLESGGGLVQPGGS(L/P)KLSCAASGFDESRYW(M/R)
SWVRQAPGK(G/E)LEWIGEIHPYSSTINYTPSVKDK(F/L)IISRD
(N/S)AKNTLYLQMSEVRSEDTALYYCARAFAYWGQGTLVTVSA

In some embodiments, the antibody or antigen-binding fragment thereof comprises or consists of the following consensus heavy chain sequence in which the most common amino acids are indicated at each position (SEQ ID NO: 22) (variable domain is highlighted

(SEQ ID NO: 22)
EVKLLESGGGLVQPGGSLKLSCAASGFDESRYWMSWVRQAPGKGLEWIGE
IHPYSSTINYTPSVKDKFIISRDNAKNTLYLQMSEVRSEDTALYYCARAF
AYWGQGTLVTVSAAKTTPPSVYPLAPGSAAQTNSMVTLGCLVKGYF;

In some embodiments, the antibody or antigen-binding fragment thereof comprises or consists of the following consensus variable heavy chain sequence in which the most common amino acids are indicated at each position (SEQ ID NO: 15):
EVKLLESGGGLVOPGGSLKLSCAASGFDFSRYWMSWVRQAPGKGLEWIGEIHPYSSTINYTPSVKDKFI ISRDNAKNTLYLQMSEVRSEDTALYYCARAFAYWGQGTLVTVSA (SEQ ID NO: 15).

In some embodiments, the antibody or antigen-binding fragment thereof comprises or consists of the following variable light chains:

a.
(SEQ ID NO: 23; also referred to herein as the V_{L} chain
of the parental mouse mAb clone “58/8/31/1/13”)
DIVMTQTPLTLSVTIGQPASISCKSSQSLLDSDGKTYINWLLQRPGQSPKRLIYLVSKLDSGVPDRETG
SGSGTDFTLKISRVEAEDLGVYYCWQGTHFPYTFGGGTKVEIKR;
b.
(SEQ ID NO: 23; also referred to herein as “VL0”)
DIVMTQTPLTLSVTIGQPASISCKSSQSLLDSDGKTYLNWLLQRPGQSPKRLIYLVSKLDSGVPDRFTG
SGSGTDFTLKISRVEAEDLGVYYCWQGTHFPYTFGGGTKVEIKR;
c.
(SEQ ID NO: 24; also referred to herein as “VL1”)
DVVMTQSPLSLPVTLGQPASISCKSSQSLLDSDGKTYLNWFQQRPGQSPRRLIYLVSKLDSGVPDRESG
SGSGTDFTLKISRVEAEDVGVYYCWQGTHFPYTFGGGTKVEIKR;
d.
(SEQ ID NO: 25; also referred to herein as “VL2”)
DVVMTQSPLSLPVTLGQPASISCRSSQSLLDSDGKTYINWELQRPGQSPRRLIYLVSKLDSGVPDRESG
SGSGTDFTLKISRVEAEDVGVYYCWQGTHEPYTFGQGTKVEIKR;
e.
(SEQ ID NO: 26; also referred to herein as “VL3”)
DIVMTQTPLSLSVTPGQPASISCKSSQSLLDSDGKTYLNWYLQKPGQSPQLLIYLVSKLDSGVPDRESG
SGSGTDFTLKISRVEPEDVGVYYCWQGTHFPYTFGGGTKVEVKR;
f.
(SEQ ID NO: 27; also referred to herein as “VL4”)
DIVMTQTPLSSPVTLGQPASISCRSSQSLLDSDGKTYLNWLQQRPGQPPRLLIYLVSKLDSGVPDRESG
SGAGTDFTLKISRVEAEDVGVYYCWQGTHEPYTEGQGTKLEIKR;
or
g.
(SEQ ID NO: 28; also referred to herein as “VL5”)
DIVMTQTPLSLSVTPGQPASISCKSSQSLLDSDGKTYLNWYLQKPGQSPQLLIYLVSKLDSGVPDRFSG
SGSGTDFTLKISRVEAEDVGVYYCWQGTHFPYTFGGGTKVEIKR.

In some embodiments, the antibody or antigen-binding fragment thereof comprises or consists of the following consensus variable light chain sequence, wherein substitutions or deletions are particular positions are indicated in parenthesis, deletions being marked as ‘X’:

(SEQ ID NO: 29)
D(I/V)VMTQ(T/S)PL(T/S)(L/S)(S/P)VT(I/L/P)GQPASISC
(K/R)SSQSLLDSDGKTYLNW(L/F/E/Y)(L/Q)Q(R/K)PGQ(S/P)
P(K/R/Q)(R/L)LIYLVSKLDSGVPDRF(T/S)GSG(S/A)GTDFTLK
ISRVE(A/P)ED(L/V)GVYYCWQGTHEPYTFG(G/Q)GTK(V/L)E
(I/V)KR

In some embodiments, the antibody or antigen-binding fragment thereof comprises or consists of the following consensus variable chain sequence in which the most common amino acids are indicated at each position (SEQ ID NO: 30) (variable domain is highlighted in bold, and CDRs double underlined, as determined by IMGT):

(SEQ ID NO: 30)
DIVMTQTPLTLSVTIGQPASISCKSSQSLLDSDGKTYLNWLLQRPGQSPK
RLIYLVSKLDSGVPDRFTGSGSGIDFTLKISRVEAEDLGVYYCWQGTHEP
YTFGGGTKVEIKRADAAPTVSIFPPSSEQLTSGGASVVCFLNNFYPR;

In some embodiments, the antibody or antigen-binding fragment thereof comprises or consists of the following consensus variable light chain sequence in which the most common amino acids are indicated at each position (SEQ ID NO: 31):

(SEQ ID NO: 31)
DIVMTQTPLTLSVTIGQPASISCKSSQSLLDSDGKTYLNWLLQRPGQSPK
RLIYLVSKLDSGVPDRFTGSGSGTDFTLKISRVEAEDLGVYYCWQGTHEP
YTFGGGTKVEIKRADAAPTVSIFPPSSEQL.

In some embodiments, the variable heavy chain sequence and/or the variable light chain sequence further comprises a signal sequence, for example peptide MGWTLVFLFLLSVTAGVHS (SEQ ID NO: 32).

Accordingly, antibodies or antigen-binding fragments thereof can be formed by combining any VH specified above with any VL specified above. For example, VH4 or VH5 may be combined with VL2, as follows (which may optionally comprise a linker, for example (G4S) 3, between the two domains):

VH4-VL2
(SEQ ID NO: 33)
EVQLVESGGGLVQPGGSLRLSCAASGEDFSRYWMSWVRQAPGKGLEWVAEIHPYSSTINYTPSVKDRFT
ISRDNAKNSLYLQMNSLRSEDTAVYYCARAFAYWGQGTLVTVSS[linker]DVVMTQSPLSLPVTLGQ
PASISCRSSQSLLDSDGKTYLNWELQRPGQSPRRLIYLVSKLDSGVPDRFSGSGSGTDFTLKISRVEAE
DVGVYYCWQGTHEPYTFGQGTKVEIKR
VH5-VL2
(SEQ ID NO: 34)
EVQLVESGGGLVQPGGSLRLSCAASGEDFSRYWMSWVRQAPGKGLEWVSEIHPYSSTINYTPSVKDRFT
ISRDNAKNSLYLQMNSLRAEDTAVYYCARAFAYWGQGTLVTVSA[linker]DVVMTQSPLSLPVTLGQ
PASISCRSSQSLLDSDGKTYLNWELQRPGQSPRRLIYLVSKLDSGVPDRFSGSGSGTDFTLKISRVEAE
DVGVYYCWQGTHEPYTFGQGTKVEIKR

It will be understood that any of the VH domains may be combined with any of the VL domains. In some embodiments, VH0 is combined with VL0, VL1, VL2, VL3, VL4 or VL5.

In some embodiments, VH1 is combined with VL0, VL1, VL2, VL3, VL4 or VL5. In some embodiments, VH2 is combined with VL0, VL1, VL2, VL3, VL4 or VL5, In some embodiments, VH3 is combined with VL0, VL1, VL2, VL3, VL4 or VL5, In some embodiments, VH4 is combined with VL0, VL1, VL2, VL3, VL4 or VL5. In some embodiments, VH5 is combined with VL0, VL1, VL2, VL3, VL4 or VL5.

In some embodiments, the antibody or antigen-binding fragment thereof further comprises a constant domain. For example, the constant domain may correspond to the following sequence:

### Heavy Chain Constant Domain:

(SEQ ID NO: 35)
ASTKGPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLMISR
TPEVTCVVVDVSHEDPEVKENWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSLSPGK
Light chain constant domain:
(SEQ ID NO: 36)
TVAAPSVFIFPPSDEQLKSGTASVVCLINNFYPREAKVQWKVDNALQSGNSQESVTEQDSKDSTYSLSS
TLTLSKADYEKHKVYACEVTHQGLSSPVTKSENRGEC

Where a VH # or VL # further comprises the corresponding heavy or light chain constant domain, it is referred to as HC # or LC #, respectively. For example, taking VH4 and including the heavy chain constant domain results in HC4; and taking VL2 and Including the light chain constant domain results in LC2. Combining the heavy and light chain domains for this example therefore results in HC4-LC2 (also referred to herein as “HC4LC2” or “HC4 LC2”, all of which are used interchangeably).

In some embodiments, the antibody or antigen-binding fragment thereof comprises or consists of the VH4-VL2 CDR sequences, and/or comprises or consists of the VH4-VL2 VH and/or VL sequences, optionally further comprising a heavy chain constant domain and/or light chain constant domain as specified herein (i.e. the antibody or antigen-binding fragment thereof may comprise or consist of the HC4-LC2 sequences). In some embodiments, the antibody or antigen-binding fragment thereof comprises or consists of the VH5-VL2 CDR sequences, and/or comprises or consists of the VH5-VL2 VH and/or VL sequences, optionally further comprising a heavy chain constant domain and/or light chain constant domain as specified herein (i.e. the antibody or antigen-binding fragment thereof may comprise or consist of the HC5-LC2 sequences).

In some embodiments, the antibody or antigen-binding fragment thereof does not have specificity for any other VEGF splice variants not containing exon 8b. An example of a VEGF splice variant that does not contain exon 8b is VEGF-A₁₆₅a. Therefore, in some embodiments, the antibody or antigen-binding fragment thereof does not have specificity for VEGF-A₁₆₅a.

In some embodiments, the antibody or antigen-binding fragment thereof further comprises a moiety. The moiety may be an amino acid sequence motif, optionally contained in the heavy chain constant region. The moiety may be for increasing the in vivo half-life of the antibody or antigen-binding fragment thereof. The moiety may be selected from the group of post translational modifications consisting of polyethylene glycol (PEG), glycosylation, fatty acids, and dextran. The moiety may be a genetic fusion protein. For example, the genetic fusion protein may be to human serum albumin. Additionally, or alternatively, the genetic fusion protein may be to a cytokine, which may form an immunocytokine. In some embodiments, the moiety is a half-life extension motif, optionally wherein the half-life extension motif is contained in the Fc domain and not the variable domain.

In some embodiments, the antibody or antigen-binding fragment thereof is PEGylated.

In some embodiments, the moiety is a cytotoxic moiety. For example, the cytotoxic moiety may comprise or consist of a radioisotope, such as a radioisotope selected from the group consisting of Astatine-211, Bismuth-212, Bismuth-213, Iodine-131, Yttrium-90, Lutetium-177, Samarium-153 and Palladium-109. Alternatively, or additionally, the cytotoxic moiety may comprise or consist of a toxin, such as, but not limited to, saporin or calicheamicin. Alternatively, or additionally, the cytotoxic moiety may comprise or consist of a chemotherapeutic agent, such as an antimetabolite.

In some embodiments, the moiety is a detectable moiety. For example, the detectable moiety may comprise or consist of a radioisotope, such as a radioisotope selected from the group consisting of Technetium-99m; Indium-111; Gallium-67; Gallium-68; Arsenic-72; Zirconium-89; Iodine-12; Thallium-201. Alternatively, or additionally, the detectable moiety may comprise or consist of a paramagnetic isotope, such as a paramagnetic isotope selected from the group consisting of Gadolinium-157; Manganese-55, Dysprosium-162, Chromium-52; Iron-56.

A second aspect of the invention relates to a pharmaceutical composition comprising an effective amount of the antibody or antigen-binding fragment thereof according to the first aspect of the Invention, and a pharmaceutically-acceptable diluent, carrier or excipient.

In some embodiments, the pharmaceutical composition is adapted for a particular delivery route. For example, the delivery may be subcutaneous, intravenous, intramuscular, intracranial, or intraocular delivery.

A third aspect of the invention relates to a kit comprising the antibody or antigen-binding fragment thereof according to the first or second aspects of the invention. In some embodiments, the kit further comprises instructions for use. Any one or more part of the kit may be stored within a vial in a form in which reconstitution and/or mixing with other components is required prior to use. Any one or more part of the kit may be in lyophilised form.

A fourth aspect of the invention relates to the antibody or antigen-binding fragment thereof according to the first aspect of the invention, or the pharmaceutical composition according to the second aspect of the invention, for use as a medicament.

A fifth aspect of the invention relates to a use of the antibody or antigen-binding fragment thereof according to the first aspect of the invention, or the pharmaceutical composition according to the second aspect of the invention, for the manufacture of a medicament for the treatment of a disease, syndrome or condition as specified herein.

A sixth aspect of the invention relates to the antibody or antigen-binding fragment thereof according to the first aspect of the invention, or the pharmaceutical composition according to the second aspect of the invention, for use in treating or preventing a disease, syndrome or condition as specified herein.

A seventh aspect of the invention relates to a method of treatment or diagnosis, wherein the method comprises administering an effective amount of the antibody or antigen-binding fragment thereof according to the first aspect of the invention, or the pharmaceutical composition according to the second aspect of the invention.

An eighth aspect of the invention relates to a method of diagnosis, wherein the method comprises subjecting a sample to the antibody or antigen-binding fragment thereof according to the first aspect of the invention, or the pharmaceutical composition according to the second aspect of the invention. In some embodiments, the method is an in vitro or ex vivo method. In some embodiments, the sample is an isolated sample, such as a sample isolated from a subject. In some embodiments, the sample is selected from the group consisting of a bodily fluid, cell, population of cells, tissue, organ, plasma and serum.

In some embodiments, the disease, syndrome or condition is selected from the group consisting of:
VEGF-associated diseases (preferably VEGF-A, even more preferably VEGF-A₁₆₅b);
Ischemia (peripheral, intestinal/mesenteric, coronary/cardiac, cerebral/brain, retinal, limb, or renal);

Peripheral arterial disease (PAD);

Atherosclerosis;

Conditions associated with diabetes (such as diabetic retinopathy, diabetic nephropathy, diabetic neuropathic pain, diabetic neuropathy);
Sclerosis (e.g. systemic sclerosis/scleroderma);
Raynaud's syndrome;
Arthritis (e.g. rheumatoid arthritis, psoriatic arthritis, osteoarthritis);
Ischemia-associated cutaneous conditions (such as cyanosis and gangrene);
Retinal ischemic diseases (such as rhegmatogenous retinal detachment and proliferative vitreoretinopathy, retinal ischemia, central vein occlusion, branch vein occlusion, nonproliferative diabetic retinopathy);
Lung conditions associated with altered angiogenesis (e.g. Asthma, Pulmonary hypertension, Idiopathic Pulmonary Fibrosis, Chronic Obstructive Pulmonary Disease);
Inflammatory bowel disease (such as ulcerative colitis and Crohn's disease);
Neuronal ischemic conditions (e.g. chronic pain, peripheral neuropathy, traumatic neuropathy, chemotherapy induced peripheral neuropathy);
Stroke;
Pre-eclampsia;
Hypertension; Obesity;
Hair loss;
Kidney failure (e.g. IgA nephropathy, hereditary kidney conditions, e.g. Denys-Drash or Frasier Syndromes, chronic kidney disease, acute kidney disease, glomerular nephropathy);
Angiogenesis/revascularisation (including tumour-associated angiogenesis and VEGF-A₁₆₅a-mediated angiogenesis);
Cancer (e.g. solid tumours);
Deep vein thrombosis (DVT);
Refractory angina; and
Myocardial infarction (MI), or post-MI conditions,

### DETAILED DESCRIPTION OF THE INVENTION

All publications, patents and patent applications cited herein, whether supra or infra, are hereby incorporated by reference in their entirety.

It is to be understood that different applications of the disclosed antibodies, uses, methods, pharmaceutical compositions and kits may be tailored to the specific needs in the art. It is also to be understood that the terminology used herein is for the purpose of describing particular embodiments of the invention only and is not intended to be limiting.

In addition, as used in this specification and the appended claims, the singular forms “a”, “an”, and “the” include plural referents unless the content clearly dictates otherwise. Thus, for example, reference to “an antibody” includes “antibodies”, reference to “an antigen” includes two or more such antigens, reference to “a subject” includes two or more such subjects, and the like.

The present invention relates to an antibody or antigen-binding fragment thereof with specificity for a splice variant of vascular endothelial growth factor (VEGF). A number of splice variants of VEGF are known. Preferably, the splice variant is VEGF-A₁₆₅b or any VEGF sequence containing the sequence encoded by Exon 8b of the VEGF gene (such as VEGF-Ax, isoform 15 of VEGF-A, and VEGF₁₆₅b). By “splice variant”, we include the meaning that the gene that encodes VEGF may be alternatively spliced to result in different isoforms or versions that translate into alternative VEGF molecules.

Advantageously, the described anti-VEGF-A₁₆₅b antibodies are effective in reversing anti-angiogenic effects of monocytes from patients with peripheral arterial disease in vitro and in vivo. It was desired to produce a panel of humanised antibodies with an affinity <10 nM, and that can block VEGF-A₁₆₅b mediated suppression of human VEGF-A₁₆₅a mediated angiogenesis in endothelial cells in culture. The present invention includes a chimeric antibody (mouse variable domains VH and VL and human heavy and light chain constant domains, HC0LC0) and humanised versions of the antibody to VEGF-A₁₆₅b. The chimeric antibody has an affinity of 300 PM and the humanised versions have affinities of 600 μM (2 clones—HC4LC2 and HC5LC2), 900 μM (HC4 LC1), 5.0 nM (HCl LC1), 2.1 nM (HCl LC2), 4.4 nM (HC2 LC1), 3.6 nM (HC2 LC2), 1.5 nM (HC3 LC1), 1.17 nM (HC3 LC2) and 3.2 nM (HC5 LC1). Three of these antibodies have been tested in cell assays and were shown to have significantly higher potency (IC₅₀ HC0 LC0 0.44 μg/ml, HC4 LC2 0.137 g/ml, HC5 LC2 0.114 μg/ml) than the parental mouse antibody (IC₅₀>1 μg/ml). This represents a significant advancement in the antibody targeting of VEGF-A₁₆₅b and provides a potential new therapy for diseases associated with poor vasculature, such as ischemia.

In some embodiments, the antibody or antigen-binding fragment thereof has an affinity of 100 to 1000 μM, for example, 200 to 800 μM, preferably 300 to 600 μM. In some embodiments, the affinity is about 100 μM, 200 μM, 300 μM, 400 μM, 500 μM, 600 μM, 700 μM, 800 μM, 900 μM, 1 nM, 1.17 nM, 1.5 nM, 2.1 nM, 3.2 nM, 3.6 nM, 4.4 nM, 5 nM, or higher. By “higher” with respect to affinity, we mean that the affinity is increased. For example, 1 μM is a higher (i.e. increased, improved, better, greater) affinity than 1 nM.

The antibody or antigen-binding fragment thereof may be specific for VEGF-A₁₆₅b. The antibody or antigen-binding fragment thereof may not bind, or may not significantly bind, VEGF-A₁₆₅a. Preferably, the antibody or antigen-binding fragment thereof binds VEGF-A₁₆₅b and does not bind, or does not significantly bind, VEGF-A₁₆₅a or any other splice variant of VEGF that uses the proximal splice site in exon 8 encoding CDKPRR. The antibody may bind VEGF-A₁₆₅b with at least 50× or 100× greater affinity than VEGF-A₁₆₅a or any other exon 8 proximal splice variant of VEGF. In some embodiments, the antibody or antigen-binding fragment thereof does not bind if the splice variant excludes exon 8b.

In some embodiments, the antibody or antigen-binding fragment thereof may have an affinity/dissociation constant (k_{D}) for VEGF-A₁₆₅b binding that is less than 1 nanomolar. The antibody or antigen-binding fragment thereof may have an affinity/dissociation constant (k_{D}) for VEGF-A₁₆₅b binding that is less than about 0.8 nM. In another embodiment, the antibody or antigen-binding fragment thereof may have an affinity/dissociation constant (k_{D}) for VEGF-A₁₆₅b binding that is about 0.6 nM (600 μM) or less. The antibody or antigen-binding fragment thereof may have an affinity/dissociation constant (k_{D}) for VEGF-A₁₆₅b binding that is between about 0.3 nM and about 0.8 nM. The antibody or antigen-binding fragment thereof may have an affinity/dissociation constant (k_{D}) for VEGF-A₁₆₅b binding that is between about 0.5 nM and about 0.7 nM. The antibody or antigen-binding fragment thereof may have an affinity/dissociation constant (k_{D}) for VEGF-A₁₆₅b binding that is between about 0.55 nM and about 0.65 nM.

In some embodiments, the antibody or antigen-binding fragment thereof has one or more of the following properties:
a. reduces the affinity of VEGF-A₁₆₅b binding to VEGF receptor 2 (VEGFR2);
b. blocks VEGF-A₁₆₅b mediated inhibition of cell migration, for example when tested in vitro, in which the antibody or antigen-binding fragment thereof effectively inhibits cell migration comparable to or better than the parental mouse monoclonal and chimeric antibody as a control;
c. reverses diabetic peripheral arterial disease (PAD) human monocyte mediated inhibition of endothelial migration (for example as per the assay described in Example 4 in comparison with a control).

The present invention advantageously provides an antibody or antigen-binding fragment thereof that has a significantly high specificity for VEGF-A₁₆₅b in the picomolar range, and it is humanized for efficacy and safety in human treatment.

In a preferred embodiment, the VEGF-A₁₆₅b target of the antibody or antigen-binding fragment thereof is human VEGF-A₁₆₅b. The sequence of VEGF-A₁₆₅b may comprise of the C-terminal sequence of TCRSLTRKD (SEQ ID NO: 37). The antibody or antigen-binding fragment thereof may have specific affinity for a peptide comprising or consisting of the sequence TCRSLTRKD (SEQ ID NO: 37), optionally coupled to keyhole limpet hemocyanin (KLH). The sequence TCRSLTRKD (SEQ ID NO: 37) corresponds to the final 9 amino acids of human VEGF165b, which was used as an immunogen to generate the antibodies.

Variants of VEGF-A that fully or partially contain the TCRSLTRKD sequence include VEGFA_HUMAN Isoform 15 of Vascular endothelial growth factor A and vascular endothelial growth factor A isoform VEGF-Ax precursor, as follows:

>sp|P15692-15|VEGFA HUMAN Isoform 15 of Vascular endothelial growth
factor A OS = Homo sapiens OX = 9606 GN = VEGFA
(SEQ ID NO: 159)
MTDRQTDTAPSPSYHLLPGRRRTVDAAASRGQGPEPAPGGGVEGVGARGVALKLFVQLLGCSREGGAVV
RAGEAEPSGAARSASSGREEPQPEEGEEEEEKEEERGPQWRLGARKPGSWTGEAAVCADSAPAARAPQA
LARASGRGGRVARRGAEESGPPHSPSRRGSASRAGPGRASETMNFLLSWVHWSLALLLYLHHAKWSQAA
PMAEGGGQNHHEVVKFMDVYQRSYCHPIETLVDIFQEYPDEIEYIFKPSCVPLMRCGGCCNDEGLECVP
TEESNITMQIMRIKPHQGQHIGEMSFLQHNKCECRPKKDRARQENPCGPCSERRKHLFVQDPQTCKCSC
KNTDSRCKARQLELNERTCRSLTRKD
>NP_001303939.1 vascular endothelial growth factor A isoform VEGF-Ax
precursor [Homo sapiens]
(SEQ ID NO: 160)
MNFLLSWVHWSLALLLYLHHAKWSQAAPMAEGGGQNHHEVVKFMDVYQRSYCHPIETLVDIFQEYPDEI
EYIFKPSCVPLMRCGGCCNDEGLECVPTEESNITMQIMRIKPHQGQHIGEMSFLQHNKCECRPKKDRAR
QENPCGPCSERRKHLFVQDPQTCKCSCKNTDSRCKARQLELNERTCRCDKPRRSAGQEEGASLRVSGTR
SLTRKD

The term “antibody” as referred to herein includes whole antibodies and any antigen binding portion (i.e. “antigen-binding fragment”) or single chains thereof. An antibody refers to a glycoprotein comprising at least two heavy (H) chains and two light (L) chains inter-connected by disulfide bonds, or an antigen-binding portion thereof. Each heavy chain is comprised of a heavy chain variable region (abbreviated herein as VH) and a heavy chain constant region. Each light chain is comprised of a light chain variable region (abbreviated herein as VL) and a light chain constant region. The variable regions of the heavy and light chains contain a binding domain that interacts with an antigen. The VH and VL regions can be further subdivided into regions of hypervariability, termed complementarity determining regions (CDR), interspersed with regions that are more conserved, termed framework regions (FR). The constant regions of the antibodies may mediate the binding of the immunoglobulin to host tissues or factors, including various cells of the immune system (e.g. effector cells) and the first component (C1q) of the classical complement system.

Heavy chains can be of any isotype, including IgG (IgG1, IgG2, IgG3 and IgG4 subtypes), IgA (IgA1 and IgA2 subtypes), IgM and IgE. Light chains include kappa chains and lambda chains.

Therefore, in some embodiments, the variable regions (VH and VL) of antibodies of the present invention may further comprise (e.g. be linked to) antibody constant regions or parts thereof. For example, the variable regions of antibodies may be attached at their C-terminal end to antibody light chain constant domains including human Ck or CA chains. Similarly, the variable regions of antibodies may be attached at their C-terminal end to all, or part of, an immunoglobulin heavy chain derived from any antibody isotype, e.g. IgG, IgA, IgE and IgM and any of the isotype sub-classes, particularly IgG1, IgG2 and IgG4.

Of relevance are antibodies and their antigen-binding fragments that have been “isolated” so as to exist in a physical milieu distinct from that in which it may occur in nature or that have been modified so as to differ from a naturally occurring antibody in amino acid sequence.

The term “isolated” refers to the state in which specific antibodies or antigen-binding fragments of the invention, or nucleic acid encoding such, will preferably be, in accordance with the present invention. Antibodies and antigen-binding fragments thereof, and nucleic acids, will generally be free or substantially free of material with which they are naturally associated such as other polypeptides (e.g. host cell proteins) or nucleic acids with which they are found in their natural environment, or the environment in which they are prepared (e.g., cell culture) when such preparation is by recombinant DNA technology practised in vitro or in vivo. Specific monoclonal antibodies and nucleic acid may be formulated with diluents or adjuvants or excipients and still for practical purposes be isolated—for example, the members will normally be mixed with gelatin or other carriers if used to coat microtitre plates for use in immunoassays, or will be mixed with pharmaceutically acceptable carriers, diluents and/or excipients when used in diagnosis or therapy. Specific binding antibodies or antigen-binding fragments thereof may be glycosylated, either naturally or by systems of heterologous eukaryotic cells, or they may be aglycosylated (for example if produced by expression in a prokaryotic cell or mammalian cells deficient in specific glycosylation pathways or if the antibody or antigen binding fragment has been specifically engineered to remove glycosylation sites).

An antibody or antigen-binding portion thereof may be a polyclonal antibody or a monoclonal antibody. The antibody or antigen-binding portion thereof may be produced by any suitable method. For example, suitable methods for producing monoclonal antibodies are disclosed in “Monoclonal Antibodies; A manual of techniques”, H Zola (CRC Press, 1988) and in “Monoclonal Hybridoma Antibodies: Techniques and Application”, SGR Hurrell (CRC Press, 1982). Recombinant techniques may also be used.

The term “antigen-binding portion” or “antigen-binding fragment” of an antibody refers to one or more fragments of an antibody that retain the ability to specifically bind to an antigen, such as VEGF-A₁₆₅b. It has been shown that the antigen-binding function of an antibody can be performed by fragments of a full-length antibody. Examples of binding fragments encompassed within the term “antigen-binding portion” of an antibody include a Fab fragment, a F(ab′) 2 fragment, a Fab′ fragment, a Fd fragment, a Fv fragment, and a dAb fragment. Single chain antibodies such as scFv and heavy chain antibodies such as VHH and camelid antibodies termed nanobodies are also intended to be encompassed within the term “antigen-binding portion” of an antibody. These antibody fragments may be obtained using conventional techniques known to those of skill in the art, and the fragments may be screened for utility in the same manner as intact (i.e. full length) antibodies.

An antibody for use in the methods of the invention may be a human antibody. The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g. mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences-such antibodies are typically referred to as chimeric or humanised.

Methods for humanising non-human antibodies are well known in the art. Generally, the humanised antibody has one or more amino acid residues introduced into it from a source which is non-human. These non-human amino acid residues, often referred to as imported residues, are typically taken from an imported variable domain. Humanisation can be essentially performed as described (see, for example, Jones et al., 1986, Nature 321:522-525; Reichmann et al., 1988. Nature 332:323-327; Verhoeven et al., 1988, Science 239:1534-15361; U.S. Pat. No. 4,816,567, the disclosures of which are incorporated herein by reference) by substituting human complementarity determining regions with the corresponding rodent complementarity determining regions. In practice, humanised antibodies may be typically human antibodies in which some complementarity determining region residues and possibly some framework residues are substituted by residues from analogous sites in rodent (e.g. mice) antibodies. For example, a chimeric antibody can be one in which the VH and VL of the rodent antibody are fused to a human IgG constant region (CH and CL or CK), A humanised antibody, on the other hand, is one in which the CDRs from the rodent antibody replace the CDRs in a human antibody framework which has a high level of sequence identity/homology to the parental rodent antibody. This process is known as CDR grafting. It will be understood that such antibodies are not limited to analogous sites in rodent antibodies. Rather, the analogous sites may be from other non-human parental antibody species, for example including rabbits, llamas, monkeys, etc.

A human antibody for use the methods of the Invention is typically a human monoclonal antibody. Such a human monoclonal antibody may be produced by a hybridoma which includes a B cell obtained from a transgenic nonhuman animal, e.g. a transgenic mouse, having a genome comprising a human heavy chain transgene and a light chain transgene fused to an immortalised cell. Human antibodies may also be prepared by in vitro Immunisation of human lymphocytes followed by transformation of the lymphocytes with Epstein-Barr virus. The term “human antibody derivatives” refers to any modified form of the human antibody, e.g. a conjugate of the antibody and another agent or antibody.

An antibody or antigen-binding portion thereof according to the invention may alternatively be a humanised antibody.

The term “humanised” refers to an antibody molecule, generally prepared using recombinant techniques, having an antigen binding site derived from an immunoglobulin from a non-human species and a remaining immunoglobulin structure based upon the structure and/or sequence of a human immunoglobulin.

In one embodiment the antigen-binding site may comprise complete non-human antibody variable domains fused to human constant domains, in which case the antibody is “chimeric”. Chimeric antibodies are discussed by Neuberger et al (1998, 8^{th} International Biotechnology Symposium Part 2, 792-799).

Alternatively, in fully humanised antibodies (described as “humanised” herein) only the complementarity determining regions (CDRs) of such variable domains are grafted into the appropriate human framework regions of human variable domains. The framework residues of such humanised molecules may be wild type (e.g. fully human) or they may be modified to contain one or more amino acid substitutions not found in the human antibody whose sequence has served as the basis for humanisation. Humanisation lessens or eliminates the likelihood that the ‘humanised antibody’ will act as an immunogen in human individuals, but the possibility of an immune response to the foreign variable region remains (LoBuglio, A. F. et al. (1989) “Mouse/Human Chimeric Monoclonal Antibody In Man: Kinetics And Immune Response,” Proc. Natl. Acad. Sci. (U.S.A.) 86:4220-4224).

It will be appreciated by persons skilled in the art that for human therapy or diagnostics, human or humanised antibodies are preferably used. Humanised forms of non-human (e.g. murine) antibodies are genetically engineered chimeric antibodies or antibody fragments having preferably minimal amino acid residues derived from non-human antibodies. Humanised antibodies include antibodies in which complementary determining regions of a human antibody (recipient antibody) are replaced by residues from a complementary determining region of a non-human species (donor antibody) such as mouse, rat or rabbit having the desired functionality. In some instances, Fv framework residues of the human antibody are replaced by corresponding non-human residues. Humanised antibodies may also comprise residues which are found neither in the recipient antibody nor in the Imported complementarity determining region or framework sequences. In general, the humanised antibody will comprise substantially all of at least one, and typically two, variable domains, in which all or substantially all of the complementarity determining regions correspond to those of a non-human antibody and all, or substantially all, of the framework regions correspond to those of a relevant human consensus sequence. Humanised antibodies optimally also include at least a portion of an antibody constant region, such as an Fc region, typically derived from a human antibody (see, for example, Jones et al., 1986. Nature 321:522-525; Riechmann et al., 1988, Nature 332:323-329; Presta, 1992, Curr. Op. Struct. Biol. 2:593-596).

Methods for humanising non-human antibodies are well known in the art. Generally, the humanised antibody has one or more amino acid residues introduced into it from a source which is non-human. These non-human amino acid residues, often referred to as imported residues, are typically taken from an imported variable domain. Humanisation can be essentially performed as described (see, for example, Jones et al., 1986, Nature 321:522-525; Reichmann et al., 1988. Nature 332:323-327; Verhoeyen et al., 1988, Science 239:1534-15361; U.S. Pat. No. 4,816,567) by substituting human complementarity determining regions with corresponding rodent complementarity determining regions. In practice, humanised antibodies may be typically human antibodies in which some complementarity determining region residues and possibly some framework residues are substituted by residues from analogous sites in rodent antibodies.

Human antibodies can also be identified using various techniques known in the art, including from phage display libraries (see, for example, Hoogenboom & Winter, 1991, J. Mol. Biol. 227:381; Marks et al., 1991, J. Mol. Biol. 222:581; Cole et al., 1985, In: Monoclonal antibodies and Cancer Therapy, Alan R. Liss, pp. 77; Boerner et al., 1991. J. Immunol. 147:86-95).

Another approach focuses not only on providing human-derived constant regions, but modifying the variable regions as well so as to reshape them as closely as possible to human form. It is known that the variable regions of both heavy and light chains contain three complementarity-determining regions (CDRs) which vary in response to the antigens in question and determine binding affinity, flanked by four framework regions (FRs) which are relatively conserved in a given species and which putatively provide a scaffold for the CDRs. When nonhuman antibodies are prepared with respect to a particular antigen, the variable regions can be “reshaped” or “humanised” by grafting CDRs derived from nonhuman antibody into the FRs present in the human antibody to be modified. Application of this approach to various antibodies has been reported by Sato, K. et al. (1993) Cancer Res 53:851-856. Riechmann, L. et al. (1988) “Reshaping Human Antibodies for Therapy,” Nature 332:323-327; Verhoeyen, M. et al. (1988) “Reshaping Human Antibodies: Grafting An Antilysozyme Activity,” Science 239:1534-1536; Kettleborough, C. A. et al. (1991) “Humanization Of A Mouse Monoclonal Antibody By CDR-Grafting: The Importance Of Framework Residues On Loop Conformation,” Protein Engineering 4:773-3783; Maeda, H. et al. (1991) “Construction Of Reshaped Human Antibodies With HIV-Neutralizing Activity,” Human Antibodies Hybridoma 2:124-134; Gorman, S. D. et al. (1991) “Reshaping A Therapeutic CD4 Antibody,” Proc. Natl. Acad. Sci. (U.S.A.) 88:4181-4185; Tempest, P. R. et al. (1991) “Reshaping A Human Monoclonal Antibody To Inhibit Human Respiratory Syncytial Virus Infection in vivo,” Bio/Technology 9:266-271; Co, M. S. et al. (1991) “Humanized Antibodies For Antiviral Therapy,” Proc. Natl. Acad. Sci. (U.S.A.) 88:2869-2873; Carter, P. et al. (1992) “Humanization Of An Anti-p185her2 Antibody For Human Cancer Therapy,” Proc. Natl. Acad. Sci. (U.S.A.) 89:4285-4289; and Co, M. S. et al. (1992) “Chimeric And Humanized Antibodies With Specificity For The CD33 Antigen,” J. Immunol. 148:1149-1154.

In some embodiments, humanised antibodies preserve all six CDR sequences (for example, a humanised mouse antibody which contains all six CDRs from the mouse antibodies). In other embodiments, humanised antibodies have one or more CDRs (one, two, three, four, five, six) which are altered with respect to the original antibody, which are also termed one or more CDRs “derived from” one or more CDRs from the original antibody. The ability to humanise an antibody is well known (see, e.g., U.S. Pat. Nos. 5,225,539; 5,530,101; 5,585,089; 5,859,205; 6,407,213; 6,881,557).

Any antibody referred to herein may be provided in isolated form or may optionally be provided linked (directly or indirectly) to another moiety. The other moiety may be a therapeutic molecule such as a cytotoxic moiety, antibiotic or a drug.

The therapeutic molecule may be directly attached, for example by chemical conjugation, to an antibody or antigen-binding fragment of the invention. Methods for conjugating molecules to such are known in the art. For example, carbodlimide conjugation (Bauminger & Wilchek (1980) Methods Enzymol. 70, 151-159) may be used to conjugate a variety of agents, including doxorubicin, to antibodies or peptides. The water-soluble carbodiimide, 1-ethyl-3-(3-dimethylaminopropyl) carbodiimide (EDC) is particularly useful for conjugating a functional moiety to a binding moiety.

Other methods for conjugating a moiety to antibodies can also be used. For example, sodium periodate oxidation followed by reductive alkylation of appropriate reactants can be used, as can glutaraldehyde cross-linking. However, it is recognised that, regardless of which method of producing a conjugate of the invention is selected, a determination must be made that the antibody maintains its targeting ability and that the functional moiety maintains its relevant function.

A cytotoxic moiety may be directly and/or indirectly cytotoxic. By “directly cytotoxic” it is meant that the moiety is one which on its own is cytotoxic. By “indirectly cytotoxic” it is meant that the moiety is one which, although is not itself cytotoxic, can induce cytotoxicity, for example by its action on a further molecule or by further action on it. The cytotoxic moiety may be cytotoxic only when intracellular and is preferably not cytotoxic when extracellular.

The antibody or antigen-binding portion thereof may be linked to a cytotoxic moiety which is a directly cytotoxic chemotherapeutic agent. Optionally, the cytotoxic moiety is a directly cytotoxic polypeptide. Cytotoxic chemotherapeutic agents are well known in the art. In this context of the present invention, the antibody or antigen-binding portion thereof with a cytotoxic chemotherapeutic agent is for use in combination with a distinct chemotherapy. In some embodiments, the antibody or antigen-binding portion thereof is not conjugated to a cytotoxic chemotherapeutic agent.

Cytotoxic chemotherapeutic agents, whether for conjugated to the antibody or antigen-binding-portion thereof or for separate use as chemotherapy, such as anticancer agents, include: alkylating agents including nitrogen mustards such as mechlorethamine (HN2), cyclophosphamide, ifosfamide, melphalan (L-sarcolysin) and chlorambucil; ethyleneimines and methylmelamines such as hexamethylmelamine, thiotepa; alkyl sulphonates such as busulfane; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin). Natural products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; and biological response modifiers such as interferon alphenomes. Miscellaneous agents including platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MIH); and adrenocortical suppressant such as mitotane (o,p′-DDD) and aminoglutethimide; taxol and analogues/derivatives; and hormone agonists/antagonists such as flutamide and tamoxifen.

The cytotoxic moiety may be a cytotoxic peptide or polypeptide moiety which leads to cell death. Cytotoxic peptide and polypeptide moieties are well known in the art and include, for example, ricin, abrin, Pseudomonas exotoxin, tissue factor and the like. Methods for linking them to targeting moieties such as antibodies are also known in the art. Other ribosome inactivating proteins are described as cytotoxic agents in WO 96/06641. Pseudomonas exotoxin may also be used as the cytotoxic polypeptide. Certain cytokines, such as TNFα and IL-2, may also be useful as cytotoxic agents.

Certain radioactive atoms may also be cytotoxic if delivered in sufficient doses. Thus, the cytotoxic moiety may comprise a radioactive atom which, in use, delivers a sufficient quantity of radioactivity to the target site so as to be cytotoxic. Suitable radioactive atoms include Phosphorus-32, Iodine-125, Iodine-131, Indium-111, Rhenium-186, Rhenium-188 or Yttrium-90, or any other isotope which emits enough energy to destroy neighbouring cells, organelles or nucleic acid. Preferably, the isotopes and density of radioactive atoms in the agents of the invention are such that a dose of more than 4000 cGy (preferably at least 6000, 8000 or 10000 cGy) is delivered to the target site and, preferably, to the cells at the target site and their organelles, particularly the nucleus.

The radioactive atom may be attached to the antibody, antigen-binding fragment, variant, fusion or derivative thereof in known ways. For example, EDTA or another chelating agent may be attached to the binding moiety and used to attach 111 In or 90Y. Tyrosine residues may be directly labelled with 1251 or 131I.

The cytotoxic moiety may be a suitable indirectly cytotoxic polypeptide. The indirectly cytotoxic polypeptide may be a polypeptide which has enzymatic activity and can convert a non-toxic and/or relatively non-toxic prodrug into a cytotoxic drug. With antibodies, this type of system is often referred to as ADEPT (Antibody-Directed Enzyme Prodrug Therapy). The system requires that the antibody locates the enzymatic portion to the desired site in the body of the patient and after allowing time for the enzyme to localise at the site, administering a prodrug which is a substrate for the enzyme, the end product of the catalysis being a cytotoxic compound. The object of the approach is to maximise the concentration of drug at the desired site and to minimise the concentration of drug in normal tissues. The cytotoxic moiety may be capable of converting a non-cytotoxic prodrug into a cytotoxic drug.

The enzyme and prodrug of the system using a targeted enzyme as described herein may be any of those previously proposed. The cytotoxic substance may be any existing anti-cancer drug such as an alkylating agent; an agent which intercalates in DNA; an agent which inhibits any key enzymes such as dihydrofolate reductase, thymidine synthetase, ribonucleotide reductase, nucleoside kinases or topoisomerase; or an agent which effects cell death by interacting with any other cellular constituent, Etoposide is an example of a topoisomerase inhibitor.

Reported prodrug systems include those listed in Table A.

TABLE A
Reported prodrug systems.

| Enzyme | Prodrug |
|---|---|
| Carboxypeptidase G2 | Derivatives of L-glutamic acid and benzoic acid |
| | mustards, aniline mustards, phenol mustards and |
| | phenylenediamine mustards; fluorinated |
| | derivatives of these |
| Alkaline phosphatase | Etoposide phosphate |
| | Mitomycin phosphate |
| Beta-glucuronidase | p-Hydroxyaniline mustard-glucuronide |
| | Epirubicin-glucuronide |
| Penicillin-V-amidase | Adriamycin-N phenoxyacetyl |
| Penicillin-G-amidase | N-(4′-hydroxyphenyl acetyl) palytoxin |
| | Doxorubicin and melphalan |
| Beta-lactamase | Nitrogen mustard-cephalosporin |
| | p-phenylenediamine; doxorubicin derivatives; |
| | vinblastine derivative-cephalosporin, |
| | cephalosporin mustard; a taxol derivative |
| Beta-glucosidase | Cyanophenylmethyl-beta-D-gluco- |
| | pyranosiduronic acid |
| Nitroreductase | 5-(Azaridin-1-yl-)-2,4-dinitrobenzamide |
| Cytosine deaminase | 5-Fluorocytosine |
| Carboxypeptidase A | Methotrexate-alanine |

Suitable enzymes for forming part of an enzymatic portion include: exopeptidases, such as carboxypeptidases G, G1 and G2 (for glutamylated mustard prodrugs), carboxypeptidases A and B (for MTX-based prodrugs) and aminopeptidases (for 2-a-aminocyl MTC prodrugs); endopeptidases, such as e.g. thrombolysin (for thrombin prodrugs); hydrolases, such as phosphatases (e.g. alkaline phosphatase) or sulphatases (e.g. aryl sulphatases) (for phosphylated or sulphated prodrugs); amidases, such as penicillin amidases and arylacyl amidase; lactamases, such as β-lactamases; glycosidases, such as β-glucuronidase (for β-glucuronomide anthracyclines), α-galactosidase (for amygdalin) and β-galactosidase (for β-galactose anthracycline); deaminases, such as cytosine deaminase (for 5FC); kinases, such as urokinase and thymidine kinase (for gancyclovir); reductases, such as nitroreductase (for CB1954 and analogues), azoreductase (for azobenzene mustards) and DT-diaphorase (for CB1954); oxidases, such as glucose oxidase (for glucose), xanthine oxidase (for xanthine) and lactoperoxidase; DL-racemases, catalytic antibodies and cyclodextrins.

Preferably, the prodrug is relatively non-toxic compared to the cytotoxic drug. Typically, it has less than 10% of the toxicity, preferably less than 1% of the toxicity as measured In a suitable in vitro cytotoxicity test.

It is likely that the moiety which is able to convert a prodrug to a cytotoxic drug will be active in isolation from the rest of the agent of the invention but it is necessary only for it to be active when (a) it is in combination with the rest of the agent of the invention and (b) the agent of the Invention is attached to, adjacent to or internalised in target cells.

When each moiety is a polypeptide, the two portions may be linked together by any of the conventional ways of cross-linking polypeptides. For example, the antibody or antigen-binding portion thereof may be enriched with thiol groups and the further moiety reacted with a bifunctional agent capable of reacting with those thiol groups, for example the N-hydroxysuccinimide ester of lodoacetic acid (NHIA) or N-succinimidyl-3-(2-pyridyldithio) propionate (SPDP). Amide and thioether bonds, for example achieved with m-maleimidobenzoyl-N-hydroxysuccinimide ester, are generally more stable in vivo than disulphide bonds.

The cytotoxic moiety may be a radiosensitiser. Radiosensitisers include fluoropyrimidines, thymidine analogues, hydroxyurea, gemcitabine, fludarabine, nicotinamide, halogenated pyrimidines, 3-aminobenzamide, 3-aminobenzodiamide, etanixadole, pimonidazole and misonidazole. Also, delivery of genes into cells can radiosensitise them, for example delivery of the p53 gene or cyclin D. The further moiety may be one which becomes cytotoxic, or releases a cytotoxic moiety, upon irradiation. For example, the boron-10 isotope, when appropriately irradiated, releases a particles which are cytotoxic. Similarly, the cytotoxic moiety may be one which is useful in photodynamic therapy such as photofrin.

By “therapeutically effective amount”, “effective amount” or “therapeutically effective”, it is meant that a given substance is administered to a subject suffering from a condition, in an amount sufficient to cure, alleviate or partially arrest the condition or one or more of its symptoms. Such therapeutic treatment may result in a decrease in severity of disease symptoms, or an increase in frequency or duration of symptom-free periods. Effective amounts for a given purpose and a given agent will depend on the severity of the disease or injury as well as the weight and general state of the subject. This may be a predetermined quantity of active antibody calculated to produce a desired therapeutic effect in association with the required additive and diluent, i.e. a carrier or administration vehicle. Further, it is intended to mean an amount sufficient to reduce or prevent a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent. A therapeutically effective amount can be determined by the ordinary skilled medical or veterinary worker based on patient characteristics, such as age, weight, sex, condition, complications, other diseases, etc., as is well known in the art.

The optimal dose can be determined by physicians based on a number of parameters including, for example, age, sex, weight, severity of the condition being treated, the active ingredient being administered and the route of administration. In general, a serum concentration of polypeptides and antibodies that permits saturation of receptors is desirable. A concentration in excess of approximately 0.1 nM is normally sufficient. For example, a dose of 100 mg/kg of antibody provides a serum concentration of approximately 20 nM for approximately eight days. As a rough guideline, doses of antibodies may be given weekly in amounts of 10-300 mg/kg.

The dose of the composition will be dependent upon the properties of the monoclonal antibody, e.g. its binding affinity and in vivo plasma half-life, the concentration of the polypeptide in the formulation, the administration route, the site and rate of dosage, target organ, the clinical tolerance of the patient involved, the pathological condition afflicting the patient and the like, as is well within the skill of the physician. For example, doses of 300 μg of antibody per patient per administration may be provided, although dosages may range from about 10 μg to 6 g per dose. Different dosages are utilised during a series of sequential inoculations; the practitioner may administer an initial inoculation and then boost with relatively smaller doses of antibody.

The term “subject”, (which herein is used interchangeably with “patient”) includes any animal, including a human, that is in need of treatment with an antibody or antigen-binding portion thereof of the present invention. The subject or patient may be mammalian or non-mammalian, Preferably, the subject is mammalian, such as a horse, or a cow, or a sheep, or a pig, or a camel, or a dog, or a cat. Most preferably, the mammalian patient is a human.

In some embodiments, the antibody or antigen-binding portion thereof is formulated and/or adapted for delivery by a route selected from the group comprising: intravenous;

intramuscular; subcutaneous; intracranial; and intraocular. For example, the antibody or antigen-binding portion thereof may be formulated and/or adapted for intravenous (i.e. “i.v” or “IV”) delivery.

In some embodiments, the antibody or antigen-binding portion thereof is delivered to the subject by a route selected from the group comprising: intravenous; intramuscular; subcutaneous; intracranial; and intraocular. For example, the antibody or antigen-binding portion thereof may be delivered intravenously.

It is envisaged that injections will be the primary route for therapeutic administration of the compositions although delivery through a catheter or other surgical tubing is also used. Some suitable routes of administration include intravenous, subcutaneous, intraperitoneal, intradermal intramuscular, intracranial and intraocular administration. Liquid formulations may be utilised after reconstitution from powder or lyophilized formulations.

For intravenous injection, or injection via other parenteral routes, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required, and as further described herein.

The composition may be administered in a localised manner or systemically.

Depending on the route of administration, the antibody or antigen-binding portion thereof may be coated in a material to protect the agent(s) from the action of acids and other natural conditions that may inactivate or denature the antibody or antigen-binding portion thereof. Preferred pharmaceutically acceptable carriers comprise aqueous carriers or diluents. Examples of suitable aqueous carriers that may be employed in the pharmaceutical compositions of the invention include water, buffered water and saline. Examples of other carriers include ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, using coating materials, such as lecithin, by the maintenance of the required particle size in the case of dispersions, and using surfactants. In many cases, it will be preferable to include isotonic agents, for example, sugars, polyalcohols such as mannitol, sorbitol, or sodium chloride in the composition.

Methods and formulations for various routes of administration are well known in the art.

The antibody or antigen-binding fragment thereof may be defined by binding affinity. The term “binding activity” is intended to refer to the tendency of an antibody molecule to bind or not to bind to a target. Binding affinity may be quantified by determining the dissociation constant (Kd) for an antibody and its target. Similarly, the specificity of binding of an antibody to its target may be defined in terms of the comparative dissociation constants (Kd) of the antibody for its target as compared to the dissociation constant with respect to the antibody and another, non-target molecule.

Typically, the Kd for the antibody with respect to the target will be 2-fold, preferably 5-fold, more preferably 10-fold lower than Kd with respect to the other, non-target molecule such as unrelated material or accompanying material in the environment. More preferably, the Kd will be 50-fold less, even more preferably 100-fold less, for example, 200-fold less, and yet more preferably 1000-fold less.

The value of this dissociation constant can be determined directly by well-known methods and can be computed even for complex mixtures by methods such as those, for example, set forth in Caceci et al. (Byte 9:340-362, 1984). For example, the Kd may be established using a double-filter nitrocellulose filter binding assay such as that disclosed by Wong & Lohman (Proc. Natl. Acad. Sci. USA 90, 5428-5432, 1993). Other standard assays to evaluate the binding ability of ligands such as antibodies towards targets are known in the art, including for example, ELISAs, Western blots, RIAs, and flow cytometry analysis. The binding kinetics (e.g., binding affinity) of the antibody also can be assessed by standard assays known in the art, such as by BIAcore™ (SPR) system analysis or biolayer interferometry (BLI) using an Octet system.

A competitive binding assay can be conducted in which the binding of the antibody to the target is compared to the binding of the target by another, known ligand of that target, such as another antibody. The concentration at which 50% inhibition occurs is known as the Ki. Under ideal conditions, the Ki is equivalent to Kd. The Ki value will never be less than the Kd, so measurement of Ki can conveniently be substituted to provide an upper limit for Kd.

The antibodies or antigen-binding fragments thereof described herein are preferably capable of binding to its target with an affinity that is at least two-fold, 10-fold, 50-fold, 100-fold or greater than its affinity for binding to another non-target molecule.

The antibody may be or may comprise a variant or a fragment of one of the specific anti-VEGF-A₁₆₅b antibodies disclosed herein, provided that said variant or fragment retains specificity for VEGF-A₁₆₅b.

A fragment is preferably an antigen-binding portion of an antibody as described herein. A fragment may be made by truncation, e.g. by removal of one or more amino acids from the N and/or C-terminal ends of a polypeptide. Up to 10, up to 20, up to 30, up to 40 or more amino acids may be removed from the N and/or C terminal in this way, Fragments may also be generated by one or more internal deletions.

A variant may comprise one or more substitutions, deletions or additions with respect to the sequences of a specific anti-VEGF-A₁₆₅b antibody disclosed herein. A variant may comprise 1, 2, 3, 4, 5, up to 10, up to 20, up to 30 or more amino acid substitutions and/or deletions from the specific sequences disclosed herein. “Deletion” variants may comprise the deletion of individual amino acids, deletion of small groups of amino acids such as 2, 3, 4 or 5 amino acids, or deletion of larger amino acid regions, such as the deletion of specific amino acid domains or other features. “Substitution” variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid.

In some embodiments, the variant comprises or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 substitutions relative to any sequence disclosed herein. In some embodiments, the variant comprises or consists of 1, 2, 3, 4, 5, 6, 7, 8, 9, 10, 11, 12, 13, 14, 15, 16, 17, 18, 19, 20, 21, 22 or 23 deletions relative to any sequence disclosed herein. In some embodiments, the substitutions and/or deletions are in the variable heavy chain sequence. In some embodiments, the substitutions and/or deletions are in the variable light chain sequence. In some embodiments, the substitutions and/or deletions are in the variable heavy chain sequence and the variable light chain sequence. In some embodiments, the substitutions and/or deletions are not in the CDR sequences of the heavy and/or light chain sequences. For example, the variant may comprise or consist of 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 substitutions in SEQ ID NOS: 19, 20 and/or 25. Alternatively, or additionally, the variant may comprise or consist of 1, 2, 3, 4, 5, 6, 7, 8, 9, 10, 11, 12, 13, 14, 15, 16, 17, 18, 19, 20, 21, 22 or 23 deletions in SEQ ID NOs: 19, 20 and/or 25.

In some embodiments, the variant comprises or consists of a percentage of sequence identity with the sequences (for example, the variable heavy chain sequences, the variable light chain sequences, and/or the CDR sequences) disclosed herein. In some embodiments, the variant comprises or consists of 99%, 98%, 97%, 96%, 95%, 94%, 93%, 92%, 91%, 90%, 85%, 80%, 75%, 70%, 65% or 60% sequence identity to the sequences (for example, the variable heavy chain sequences, the variable light chain sequences, and/or the CDR sequences) disclosed herein. In some embodiments, the variant comprises or consists of a percentage of sequence identity with the variable heavy chain sequence and/or variable light chain sequence while retaining identical CDR sequences with the corresponding variable heavy chain sequence and/or variable light chain sequence, respectively.

In some embodiments, the antibody or antigen-binding fragment thereof may comprise a variable heavy chain sequence selected from SEQ ID NOs: 15-22 or an amino acid sequence having at least 60% sequence identity therewith, for example at least 65%, 70%, 75%, 80%, 85%, 90%, 95%, or 99% sequence identity. In some embodiments, the antibody or antigen-binding fragment thereof may comprise a variable light chain sequence selected from SEQ ID NOS: 23-31 or an amino acid sequence having at least 60% sequence identity therewith, for example at least 65%, 70%, 75%, 80%, 85%, 90%, 95%, or 99% sequence identity. In some embodiments, the antibody or antigen-binding fragment thereof may comprise a CDR sequence selected from SEQ ID NOs: 1-6 or an amino acid sequence having at least 60% sequence identity therewith, for example at least 65%, 70%, 75%, 80%, 85%, 90%, 95%, or 99% sequence identity. In some embodiments, the antibody or antigen-binding fragment thereof may comprise a CDR sequence selected from SEQ ID NOS: 7-13 or an amino acid sequence having at least 60% sequence identity therewith, for example at least 65%, 70%, 75%, 80%, 85%, 90%, 95%, or 99% sequence Identity. In some embodiments, the antibody or antigen-binding fragment thereof may comprise a V_{L} CDR1 sequence selected from SEQ ID NOs: 14 or an amino acid sequence having at least 60% sequence identity therewith, for example at least 65%, 70%, 75%, 80%, 85%, 90%, 95%, or 99% sequence identity. In some embodiments, the V_{H} CDR1 sequence comprises or consists of 1, 2, 3, 4, 5 or 6 substitutions. In some embodiments, the V_{H} CDR2 sequence comprises or consists of 1, 2, 3, 4, 5, 6, 7, 8, 9, 10, 11, 12, 13, 14, 15, 16 or 17 substitutions. In some embodiments, the V_{H} CDR3 sequence comprises or consists of 1, 2, 3 or 4 substitutions. In some embodiments, the V_{L} CDR1 sequence comprises or consists of 1, 2, 3, 4, 5, 6, 7, 8, 9, 10, 11, 12, 13, 14, 15 or 16 substitutions. In some embodiments, the V_{L} CDR2 sequence comprises or consists of 1, 2, 3, 4, 5, 6 or 7 substitutions. In some embodiments, the V_{L} CDR3 sequence comprises or consists of 1, 2, 3, 4, 5, 6, 7, 8 or 9 substitutions.

In some embodiments, the antibody or antigen-binding fragment thereof comprises VH4-VL2 (SEQ ID NO: 33) or an amino acid sequence having at least 60% sequence identity therewith, for example at least 65%, 70%, 75%, 80%, 85%, 90%, 95%, or 99% sequence identity. In some embodiments, the antibody or antigen-binding fragment thereof comprises VH5-VL2 (SEQ ID NO: 34) or an amino acid sequence having at least 60% sequence identity therewith, for example at least 65%, 70%, 75%, 80%, 85%, 90%, 95%, or 99% sequence identity.

In some embodiments, the antibody or antigen-binding fragment thereof may comprise the CDRs of any one or more of the variable heavy chain sequences selected from SEQ ID NOs: 15-22. In some embodiments, the antibody or antigen-binding fragment thereof may comprise the CDRs of any one or more of the variable light chain sequences selected from SEQ ID NOs: 23-31.

Some properties of the 20 main amino acids which can be used to select suitable substituents are as follows:

| | | | |
|---|---|---|---|
| Ala (A) | aliphatic, hydrophobic, | Met (M) | hydrophobic, neutral |
| | neutral | | |
| Cys (C) | polar, hydrophobic, | Asn (N) | polar, hydrophilic, |
| | neutral | | neutral |
| Asp (D) | polar, hydrophilic, | Pro (P) | hydrophobic, neutral |
| | charged (−) | | |
| Glu (E) | polar, hydrophilic, | Gln (Q) | polar, hydrophilic, |
| | charged (−) | | neutral |
| Phe (F) | aromatic, hydrophobic, | Arg (R) | polar, hydrophilic, |
| | neutra | | charged (+) |
| Gly (G) | aliphatic, neutral | Ser (S) | polar, hydrophilic, |
| | | | neutral |
| His (H) | aromatic, polar, | Thr (T) | polar, hydrophilic, |
| | hydrophilic, | | neutral |
| | charged (+) | | |
| Ile (I) | aliphatic, hydrophobic, | Val (V) | aliphatic, hydrophobic, |
| | neutral | | neutral |
| Lys (K) | \|polar, hydrophilic, | Trp (W) | aromatic, hydrophobic, |
| | charged (+) | | neutral |
| Leu (L) | aliphatic, hydrophobic, | Tyr (Y) | aromatic, polar, |
| | neutral | | hydrophobic |

Preferred “variants” include those in which instead of the naturally occurring amino acid the amino acid which appears in the sequence is a structural analog thereof. Amino acids used in the sequences may also be derivatized or modified, e.g. labelled, providing the function of the antibody is not significantly adversely affected.

Variants may be prepared during synthesis of the antibody or by post-production modification, or when the antibody is in recombinant form using the known techniques of site-directed mutagenesis, or enzymatic cleavage and/or ligation of nucleic acids.

The invention also includes within its scope polypeptides and polynucleotides described herein and sequences having substantial identity thereto, for example, at least 70%, 80%, 85%, 90%, 95% or 99% identity thereto. In one embodiment, the CDR sequences of the sequences may be the same, but the variable regions outside of the CDR (for example, any of the VH and/or VL sequences specified herein) may be varied for example by at least 70%, 80%, 85%, 90%, 95% or 99% identity for a given variable sequence. In one embodiment, the CDR sequences of the sequences may be the same, but the framework regions may be varied for example by at least 70%, 80%, 85%, 90%, 95% or 99% identity for a given framework sequence. In another embodiment, the framework regions may be varied by 1, 2, 3, 4 or 5 amino acid modifications, such as substitutions, deletions or insertions. The variants may retain their target binding function.

The percent identity of two amino acid sequences or of two nucleic acid sequences is generally determined by aligning the sequences for optimal comparison purposes (e.g. gaps can be introduced in the first sequence for best alignment with the second sequence) and comparing the amino acid residues or nucleotides at corresponding positions. The “best alignment” is an alignment of two sequences that results in the highest percent identity. The percent identity is determined by comparing the number of identical amino acid residues or nucleotides within the sequences (I.e. % identity=number of Identical positions/total number of positions×100).

Preferably variant antibodies have an amino acid sequence which has more than 60%, or more than 70%, e.g. 75% or 80%, preferably more than 85%, e.g. more than 90% or 95% amino acid identity to the VL or VH domain of an antibody disclosed herein. This level of amino acid identity may be seen across the full length of the relevant SEQ ID NO sequence or over a part of the sequence, such as across 20, 30, 50, 75, 100, 150, 200 or more amino acids, depending on the size of the full-length polypeptide.

The determination of percent identity between two sequences can be accomplished using a mathematical algorithm known to those of skill in the art. An example of a mathematical algorithm for comparing two sequences is the algorithm of Karlin and Altschul (1990), modified as in Karlin and Altschul (1993). The NBLAST and XBLAST programs of Altschul et al. (1990) have incorporated such an algorithm. BLAST nucleotide searches can be performed with the NBLAST program, score=100, word length=12 to obtain nucleotide sequences homologous to nucleic acid molecules of the invention. BLAST protein searches can be performed with the XBLAST program, score=50, word length=3 to obtain amino acid sequences homologous to a protein molecule of the invention. To obtain gapped alignments for comparison purposes, Gapped BLAST can be utilized as described in Altschul et al. (1997). Alternatively, PSI-Blast can be used to perform an iterated search that detects distant relationships between molecules (Id.). When utilizing BLAST, GappedBLAST, and PSI-Blast programs, the default parameters of the respective programs (e.g., XBLAST and NBLAST) can be used, See http://www.ncbi.nlm.nih.gov. Another example of a mathematical algorithm utilized for the comparison of sequences is the algorithm of Myers and Miller. The ALIGN program (version 2.0) which is part of the GCG sequence alignment software package has incorporated such an algorithm. Other algorithms for sequence analysis known in the art include ADVANCE and ADAM as described in Torellis and Robotti (1994); and FASTA algorithm described in Pearson and Lipman (1988). Within FASTA, ktup is a control option that sets the sensitivity and speed of the search.

The antibodies and antigen-binding fragments thereof described herein may be modified by mutation to remove one or more sites of N-linked glycosylation (HCDR2), Met oxidation (HCDR1), Trp oxidation or Asp isomerization. Advantageously, modifying the antibodies and antigen-binding fragments thereof, for example to remove one or more sites of N-linked glycosylation (HCDR2), Met oxidation (HCDR1), Trp oxidation or Asp isomerization, may avoid liabilities arising from such post-translation modifications that can decrease antigen binding affinity, lead to product heterogeneity and aggregation, or negatively impact their expression level during production or manufacturing.

The residue numbering herein may be determined by the Kabat numbering system. For example, the heavy chains HC4 or HC5 of the antibodies described herein may comprise the mutations N59Q and/or T61A. Equivalent residues may be mutated in any of the heavy chains described herein. Additionally, or alternatively, the light chains LC1 or LC2 of the antibodies described herein may comprise one or more of the mutations selected from S32A, D33E, G34A and D60E. Equivalent residues may be mutated in any of the light chains described herein.

| Mutant chain | SEQ ID | Mutant chain | SEQ ID |
|---|---|---|---|
| HC4 | 129 | LC1 S32A | 111 |
| HC5 | 130 | LC2 S32A | 112 |
| HC4 N59Q | 104 | LC1 D33E | 113 |
| HC5 N59Q | 105 | LC2 D33E | 114 |
| HC4 T61A | 106 | LC1 G34A | 115 |
| HC5 T61A | 107 | LC2 G34A | 116 |
| LC1 | 132 | LC1 D60E | 117 |
| LC2 | 133 | LC2 D60E | 118 |

In one embodiment, the antibody or antigen-binding fragment thereof described herein may be selected from one or more of the following in which a specified mutation of the heavy chain or light chain is indicated in parenthesis following the relevant chain: HC4 (N59Q) LC1, HC4 (N59Q) LC2, HC5 (N59Q) LC2, HC4 (T61A) LC2, HC5 (T61A) LC2, HC4 LC1 (S32A), HC4 LC2 (S32A), HC5 LC2 (S32A), HC4 LC1 (D33E), HC4 LC2 (D33E), HC5 LC2 (D33E), HC4 LC1 (G34A), HC4 LC2 (G34A), HC5 LC2 (G34A).

In some embodiments, the antibody or antigen-binding fragment thereof comprises HC4 (N59Q). In some embodiments, the antibody or antigen-binding fragment thereof comprises HC5 (N59Q), In some embodiments, the antibody or antigen-binding fragment thereof comprises HC4 (T61A). In some embodiments, the antibody or antigen-binding fragment thereof comprises HC5 (T61A). In some embodiments, the antibody or antigen-binding fragment thereof comprises LC1 (S32A). In some embodiments, the antibody or antigen-binding fragment thereof comprises LC2 (S32A). In some embodiments, the antibody or antigen-binding fragment thereof comprises LC1 (D33E). In some embodiments, the antibody or antigen-binding fragment thereof comprises LC2 (D33E). In some embodiments, the antibody or antigen-binding fragment thereof comprises LC1 (G34A), In some embodiments, the antibody or antigen-binding fragment thereof comprises LC2 (G34A). In some embodiments, the antibody or antigen-binding fragment thereof comprises LC1 (D60E). In some embodiments, the antibody or antigen-binding fragment thereof comprises LC2 (D60E). In some embodiments, the antibody or antigen-binding fragment thereof comprises HC4 (D270A). In some embodiments, the antibody or antigen-binding fragment thereof comprises HC4 (P329A). In some embodiments, the antibody or antigen-binding fragment thereof comprises HC4 (P331S).

In some embodiments, the antibody or antigen-binding fragment thereof comprises a combination of any of the aforementioned mutations. For example, in some embodiments, the antibody or antigen-binding fragment thereof comprises: (i) HC4 (N59Q) and LC1 (S32A); (ii) HC4 (N59Q) and LC1 (D33E); (iii) HC4 (N59Q) and LC1 (G34A); (iv) HC4 (N59Q) and LC1 (D60E); (v) HC4 (T61A) and LC1 (S32A); (vi) HC4 (T61A) and LC1 (D33E); (vii) HC4 (T61A) and LC1 (G34A); or (viii) HC4 (T61A) and LC1 (D60E). In some embodiments the antibody or antigen-binding fragment thereof comprises: (i) HC5 (N59Q) and LC1 (S32A); (ii) HC5 (N59Q) and LC1 (D33E); (iii) HC5 (N59Q) and LC1 (G34A); (iv) HC5 (N59Q) and LC1 (D60E); (v) HC5 (T61A) and LC1 (S32A); (vi) HC5 (T61A) and LC1 (D33E); (vii) HC5 (T61A) and LC1 (G34A); or (viii) HC5 (T61A) and LC1 (D60E).

In some embodiments, a linker is present between the HC and LC domains. For example, in some embodiments, the antibody or antigen-binding fragment thereof comprises the domain combinations selected from the list consisting of: SEQ ID NO: 129+linker+SEQ ID NO: 132; SEQ ID NO: 130+linker+SEQ ID NO: 132; SEQ ID NO: 104+linker+SEQ ID NO: 132; SEQ ID NO: 105+linker+SEQ ID NO: 132; SEQ ID NO: 106+linker+SEQ ID NO: 132; SEQ ID NO: 107+linker+SEQ ID NO: 132; SEQ ID NO: 129+linker+SEQ ID NO: 133; SEQ ID NO: 130+linker+SEQ ID NO: 133; SEQ ID NO: 104+linker+SEQ ID NO: 133; SEQ ID NO: 105+linker+SEQ ID NO: 133; SEQ ID NO: 106+linker+SEQ ID NO: 133; SEQ ID NO: 107+linker+SEQ ID NO: 133; SEQ ID NO: 129+linker+SEQ ID NO: 111; SEQ ID NO: 130+linker+SEQ ID NO: 111; SEQ ID NO: 104+linker+SEQ ID NO: 111; SEQ ID NO: 105+linker+SEQ ID NO: 111; SEQ ID NO: 106+linker+SEQ ID NO: 111; SEQ ID NO: 107+linker+SEQ ID NO: 111; SEQ ID NO: 129+linker+SEQ ID NO: 112; SEQ ID NO: 130+linker+SEQ ID NO: 112; SEQ ID NO: 104+linker+SEQ ID NO: 112; SEQ ID NO: 105+linker+SEQ ID NO: 112; SEQ ID NO: 106+linker+SEQ ID NO: 112; SEQ ID NO: 107+linker+SEQ ID NO: 112; SEQ ID NO: 129+linker+SEQ ID NO: 113; SEQ ID NO: 130+linker+SEQ ID NO: 113; SEQ ID NO: 104+linker+SEQ ID NO: 113; SEQ ID NO: 105+linker+SEQ ID NO: 113; SEQ ID NO: 106+linker+SEQ ID NO: 113; SEQ ID NO: 107+linker+SEQ ID NO: 113; SEQ ID NO: 129+linker+SEQ ID NO: 114; SEQ ID NO: 130+linker+SEQ ID NO: 114; SEQ ID NO: 104+linker+SEQ ID NO: 114; SEQ ID NO: 105+linker+SEQ ID NO: 114; SEQ ID NO: 106+linker+SEQ ID NO: 114; SEQ ID NO: 107+linker+SEQ ID NO: 114; SEQ ID NO: 129+linker+SEQ ID NO: 115; SEQ ID NO: 130+linker+SEQ ID NO: 115; SEQ ID NO: 104+linker+SEQ ID NO: 115; SEQ ID NO: 105+linker+SEQ ID NO: 115; SEQ ID NO: 106+linker+SEQ ID NO: 115; SEQ ID NO: 107+linker+SEQ ID NO: 115; SEQ ID NO: 129+linker+SEQ ID NO: 116; SEQ ID NO: 130+linker+SEQ ID NO: 116; SEQ ID NO: 104+linker+SEQ ID NO: 116; SEQ ID NO: 105+linker+SEQ ID NO: 116; SEQ ID NO: 106+linker+SEQ ID NO: 116; SEQ ID NO: 107+linker+SEQ ID NO: 116; SEQ ID NO: 129+linker+SEQ ID NO: 117; SEQ ID NO: 130+linker+SEQ ID NO: 117; SEQ ID NO: 104+linker+SEQ ID NO: 117; SEQ ID NO: 105+linker+SEQ ID NO: 117; SEQ ID NO: 106+linker+SEQ ID NO: 117; SEQ ID NO: 107+linker+SEQ ID NO: 117; SEQ ID NO: 129+linker+SEQ ID NO: 118; SEQ ID NO: 130+linker+SEQ ID NO: 118; SEQ ID NO: 104+linker+SEQ ID NO: 118; SEQ ID NO: 105+linker+SEQ ID NO: 118; SEQ ID NO: 106+linker+SEQ ID NO: 118; and SEQ ID NO: 107+linker+SEQ ID NO: 118.

In some embodiments, the heavy chain is selected from one of SEQ ID NOs: 99-107. In some embodiments, the light chain is selected from one of SEQ ID NOs: 108-118. Any combination of heavy chain and light chain may be used to create an antibody or antigen-binding fragment thereof.

It can be beneficial to eliminate antibody Fc function for example, in the case of using a receptor agonist to crosslink receptors and induce signalling, or a receptor antagonist to block receptor: ligand interactions to prevent signalling. Fc engagement of Fcγ receptors on effector cells or engagement of C1q may not be wanted, as it can lead to undesired killing of biologically important cells expressing the receptor through ADCC or CDC. A single mutation of Leu235Glu in the CH2 domain of the Fc portion of human IgG1 was found to be sufficient for knocking out binding to Fc receptors on U937 cells (Wines et al., 2000, The IgG fc contains distinct fc receptor (fcr) binding sites: the leukocyte receptors fcγri and fcγriia bind to a region in the fc distinct from that recognized by neonatal for and protein a. J Immunol, May 15, 2000, 164 (10) 5313-5318). After discovery of this initial mutation, it was found that a combination of Leu234Ala and Leu235Ala (commonly called LALA mutations) eliminated FcγRIIa binding (Lund et al., 1991, Human FcγRI and FcγRII interact with distinct but overlapping sites on human IgG, J. Immunol. 147:2657; Lund et al, 1992, Multiple binding sites on the CH2 domain of IgG for mouse FcγRII, Mol. Immunol. 29:53). These two mutations were later shown to eliminate detectable binding to FcγRI, IIa, and IIIa of both IgG1 and IgG4 (Sarmay et al, 1992, Mapping and comparison of the interaction sites on the Fc region of IgG responsible for triggering antibody dependent cellular cytotoxicity (ADCC) through different types of human Fcγ receptor, Mol. Immunol. 29:633). Other mutations are known in the art, for example in Saunders K. O Conceptual Approaches to Modulating Antibody Effector Functions and Circulation Half-Life 2019 Front. Immunol., 7 Jun. 2019 Sec. Comparative Immunology.

Therefore, in one embodiment wherein the antibody or antigen-binding fragment thereof comprises an Fc portion, the antibody or antigen-binding fragment thereof according to the invention may comprise LALA (SEQ ID NO: 121) modifications in the CH2 domain of the Fc portion. The antibodies according to the invention may comprise Leu234Ala and Leu235Ala modifications in the CH2 domain of the Fc portion, or equivalent residues thereof (e.g. equivalent Leu residues which may not be numbered 234 or 235 respectively).

In one embodiment wherein the antibody or antigen-binding fragment thereof comprises an Fc portion, the antibody or antigen-binding fragment thereof according to the invention may comprise an Fc portion mutated to reduce cytopathic off-target effects on specialized epithelial cells in eye and kidney due to presence of surface bound VEGF-A₁₆₅b. For example, the Fc region may be selected from the group consisting of SEQ ID NOS: 164-166. Exemplary HC4 sequences with such Fc mutations may be selected from the group consisting of SEQ ID NOs: 161-163.

In connection with amino acid sequences, “sequence identity” refers to sequences which have the stated value when assessed using ClustalW (Thompson et al., 1994, supra) with the following parameters:
Pairwise alignment parameters-Method: accurate, Matrix: PAM, Gap open penalty: 10.00, Gap extension penalty: 0.10;
Multiple alignment parameters-Matrix: PAM, Gap open penalty: 10.00, % identity for delay: 30, Penalize end gaps: on, Gap separation distance: 0, Negative matrix: no, Gap extension penalty: 0.20, Residue-specific gap penalties: on, Hydrophilic gap penalties: on, Hydrophilic residues: GPSNDQEKR (SEQ ID NO: 120). Sequence identity at a particular residue is intended to include identical residues which have simply been derivatized.

An anti-VEGF-A₁₆₅b antibody of the invention may bind to the same epitope as a specific antibody as disclosed herein, since such an antibody is likely to mimic the action of the disclosed antibody. Whether or not an antibody binds to the same epitope as another antibody may be determined by routine methods. For example, the binding of each antibody to a target may be using a competitive binding assay. Methods for carrying out competitive binding assays are well known in the art. For example, they may involve contacting together an antibody and a target molecule under conditions under which the antibody can bind to the target molecule. The antibody/target complex may then be contacted with a second (test) antibody and the extent to which the test antibody is able to displace the first antibody from antibody/target complexes may be assessed. Such assessment may use any suitable technique, including, for example, Surface Plasmon Resonance, ELISA, or flow cytometry. The ability of a test antibody to inhibit the binding of a first antibody to the target demonstrates that the test antibody can compete with said first antibody for binding to the target and thus that the test antibody binds to the same epitope or region on the target as the first antibody, and may therefore mimic the action of the first antibody.

The invention also provides a kit comprising the antibody or antigen-binding fragment thereof according to the first aspect of the invention, or the pharmaceutical composition according to the second aspect of the invention. For example, the kit may comprise (a) a therapeutically effective amount of an antibody or antigen-binding portion thereof that specifically binds to VEGF-A₁₆₅b as described herein.

The kits of the invention may additionally comprise one or more other reagents or instruments which enable any of the embodiments mentioned above to be carried out. Such reagents or instruments include one or more of the following: suitable buffer(s) (aqueous solutions) and means to administer the anti-VEGF-A₁₆₅b antibody (such as a vessel or an instrument comprising a needle). The kit may include instructions for performing a combination therapy or method as described herein.

The anti-VEGF-A₁₆₅b antibody described herein, or provided in the kits of the invention, may be provided as a pharmaceutical composition formulated together with a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible and are also compatible with the required routes of administration.

A pharmaceutical composition may include a pharmaceutically acceptable antioxidant. These compositions may also contain excipients and adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminium monostearate and gelatin.

Pharmaceutical compositions typically must be sterile and stable under the conditions of manufacture and storage. The composition can be formulated as a solution, microemulsion, liposome, or other ordered structure suitable for high drug concentration. Sterile injectable solutions can be prepared by Incorporating the active agent (e.g. antibody) in the required amount in an appropriate solvent with one or a combination of ingredients enumerated above, as required, followed by sterilization microfiltration, Generally, dispersions are prepared by incorporating the active agent into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active agent plus any additional desired ingredient from a previously sterile-filtered solution thereof. Pharmaceutical compositions may comprise additional active ingredients as well as those mentioned above.

Suitable pharmaceutically acceptable buffers, diluents, carriers and excipients are well-known in the art (see Remington's Pharmaceutical Sciences, 18th edition, A. R Gennaro, Ed., Mack Publishing Company (1990) and handbook of Pharmaceutical Excipients, 3rd edition, A. Kibbe, Ed., Pharmaceutical Press (2000), the disclosures of which are incorporated herein by reference).

The term “buffer” is intended to include an aqueous solution containing an acid-base mixture with the purpose of stabilising pH. Examples of buffers are Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP; AMPD, AMPSO, BES, CABS, cacodylate, CHES, DIPSO, EPPS, ethanolamine, glycine, HEPPSO, imidazole, imidazolelacetic acid, PIPES, SSC, SSPE, POPSO, TAPS, TABS, TAPSO and TES.

The term “diluent” is intended to include an aqueous or non-aqueous solution with the purpose of diluting the agent in the pharmaceutical preparation. The diluent may be one or more of saline, water, polyethylene glycol, propylene glycol, ethanol or oils (such as safflower oil, corn oil, peanut oil, cottonseed oil, olive oil or sesame oil).

The term “adjuvant” is intended to include any compound added to the formulation to increase the biological effect of the agent of the invention. The adjuvant may be one or more of zinc, copper or silver salts with different anions, for example, but not limited to fluoride, chloride, bromide, iodide, tiocyanate, sulfite, hydroxide, phosphate, carbonate, lactate, glycolate, citrate, borate, tartrate, and acetates of different acyl composition. The adjuvant may also be cationic polymers such as cationic cellulose ethers, cationic cellulose esters, deacetylated hyaluronic acid, chitosan, cationic dendrimers, cationic synthetic polymers such as poly(vinyl imidazole), and cationic polypeptides such as polyhistidine, polylysine, polyarginine, and peptides containing these amino acids.

The excipient may be one or more of carbohydrates, polymers, lipids and minerals, Examples of carbohydrates include lactose, glucose, sucrose, mannitol, and cyclodextrins, which are added to the composition, e.g., for facilitating lyophilisation. Examples of polymers are starch, cellulose ethers, cellulose carboxymethylcellulose, hydroxypropylmethyl cellulose, hydroxyethyl cellulose, ethylhydroxyethyl cellulose, alginates, carrageenans, hyaluronic acid and derivatives thereof, polyacrylic acid, polysulphonate, polyethylenglycol/polyethylene oxide, polyethyleneoxide/polypropylene oxide copolymers, polyvinylalcohol/polyvinylacetate of different degree of hydrolysis, and polyvinylpyrrolidone, all of different molecular weight, which are added to the composition, e.g., for viscosity control, for achieving bioadhesion, or for protecting the lipid from chemical and proteolytic degradation. Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

The active antibody-based agents of the invention may be formulated into any type of pharmaceutical composition known in the art to be suitable for the delivery thereof.

In one embodiment, the pharmaceutical compositions of the invention may be in the form of a liposome, in which the agent is combined, in addition to other pharmaceutically acceptable carriers, with amphipathic agents such as lipids, which exist in aggregated forms as micelles, insoluble monolayers and liquid crystals. Suitable lipids for liposomal formulation include, without limitation, monoglycerides, diglycerides, sulfatides, lysolecithin, phospholipids, saponin, bile acids, and the like. Suitable lipids also include the lipids above modified by poly(ethylene glycol) in the polar headgroup for prolonging bloodstream circulation time. Preparation of such liposomal formulations can be found in for example U.S. Pat. No. 4,235,871.

The pharmaceutical compositions of the invention may also be in the form of biodegradable microspheres. Aliphatic polyesters, such as poly(lactic acid) (PLA), poly(glycolic acid) (PGA), copolymers of PLA and PGA (PLGA) or poly(caprolactone) (PCL), and polyanhydrides have been widely used as biodegradable polymers in the production of microspheres. Preparations of such microspheres can be found in U.S. Pat. No. 5,851,451 and in EP 0 213 303.

In a further embodiment, the pharmaceutical compositions of the invention are provided in the form of nanoparticles, for example based on poly-gamma glutamic acid. Details of the preparation and use of such nanoparticles can be found in WO 2011/128642. It will be appreciated by persons skilled in the art that one or more of the active components of the combination therapies of the present invention may be formulated in separate nanoparticles, or both active components may be formulated in the same nanoparticles.

In a further embodiment, the pharmaceutical compositions of the invention are provided in the form of polymer gels, where polymers such as starch, cellulose ethers, cellulose carboxymethylcellulose, hydroxypropylmethyl cellulose, hydroxyethyl cellulose, ethylhydroxyethyl cellulose, alginates, carrageenans, hyaluronic acid and derivatives thereof, polyacrylic imidazole, acid, polyvinyl polysulphonate, polyethylenglycol/polyethylene oxide, polyethyleneoxide/polypropylene oxide copolymers, polyvinylalcohol/polyvinylacetate of different degree of hydrolysis, and polyvinylpyrrolidone are used for thickening of the solution containing the agent. The polymers may also comprise gelatin or collagen.

Alternatively, the agents may simply be dissolved in saline, water, polyethylene glycol, propylene glycol, ethanol or oils (such as safflower oil, corn oil, peanut oil, cottonseed oil or sesame oil), tragacanth gum, and/or various buffers.

It will be appreciated that the pharmaceutical compositions of the invention may include ions and a defined pH for potentiation of action of the active agent. Additionally, the compositions may be subjected to conventional pharmaceutical operations such as sterilisation and/or may contain conventional adjuvants such as preservatives, stabilisers, wetting agents, emulsifiers, buffers, fillers, etc.

According to another aspect of the present invention, there is provided the antibody or antigen-binding fragment thereof according to the first aspect of the invention, or the pharmaceutical composition according to the second aspect of the invention, for use in the treatment or prevention of ischemia in a subject.

According to another aspect of the present invention, there is provided the use of the antibody or antigen-binding fragment thereof according to the first aspect of the invention, or the pharmaceutical composition according to the second aspect of the invention, in the manufacture of a medicament for the treatment or prevention of ischemia in a subject.

According to another aspect of the present invention, there is provided a method of treating or preventing ischemia, comprising administering the antibody or antigen-binding fragment thereof according to the first aspect of the invention, or the pharmaceutical composition according to the second aspect of the invention, to a subject in need thereof.

The ischemia may be the result of peripheral artery disease (PAD) (also called peripheral arterial disease). In one embodiment, the disease to be treated or prevented is a condition associated with ischemia. In one embodiment the disease to be treated or prevented is peripheral artery disease (PAD) and/or conditions associated with PAD.

The ischemia may be selected from cardiac or coronary ischemia, intestinal ischemia, brain Ischemia, limb ischemia, retinal ischemia, renal ischemia and associated cutaneous conditions such as cyanosis and gangrene.

According to another aspect of the present invention, there is provided the monoclonal antibody of the invention for use in the treatment or prevention of diseases in which VEGF-A₁₆₅b is raised relative to VEGF-A₁₆₅a, including but not limited to myocardial infarction (MI) including ST Elevated MI, angina, systemic sclerosis/scleroderma, Raynaud's syndrome, ulcerative colitis, Crohn's disease, Inflammatory bowel disease, diabetic retinopathy, diabetic nephropathy, diabetic neuropathic pain, diabetic neuropathy, stroke, pre-eclampsia, hypertension, obesity or hair loss.

According to another aspect of the present invention, there is provided the use of the antibody or antigen-binding fragment thereof according to the first aspect of the invention, or the pharmaceutical composition according to the second aspect of the invention, in the manufacture of a medicament for the treatment or prevention of diseases in which VEGF-A₁₆₅b is raised relative to VEGF-A₁₆₅a, including but not limited to myocardial infarction (MI) including ST Elevated MI, angina, systemic sclerosis/scleroderma, Raynaud's syndrome, ulcerative colitis, Crohn's disease, inflammatory bowel disease, diabetic retinopathy, diabetic nephropathy, diabetic neuropathic pain, diabetic neuropathy, stroke, pre-eclampsia, hypertension, obesity or hair loss.

According to another aspect of the present invention, there is provided a method of treating or preventing diseases in which VEGF-A₁₆₅b is raised relative to VEGF-A₁₆₅a, including but not limited to myocardial infarction (MI) including ST Elevated MI, angina, systemic sclerosis/scleroderma, Raynaud's syndrome, ulcerative colitis, Crohn's disease, inflammatory bowel disease, diabetic retinopathy, diabetic nephropathy, diabetic neuropathic pain, diabetic neuropathy, stroke, pre-eclampsia, hypertension, obesity or hair loss, comprising administering the antibody or antigen-binding fragment thereof according to the first aspect of the invention, or the pharmaceutical composition according to the second aspect of the invention, to a subject in need thereof.

A convenient way of producing an antibody or antigen-binding portion according to the present invention is to express the nucleic acid encoding it, by use of nucleic acid in an expression system.

Therefore, in further aspects, the invention provides nucleic acid which comprises a sequence encoding the antibody or antigen-binding fragment thereof according to the first aspect of the invention, or the heavy chain and/or light chain thereof.

The present invention further provides an isolated nucleic acid encoding a specific monoclonal antibody of the present invention. Nucleic acid Includes DNA and RNA. In a preferred aspect, the present invention provides a nucleic acid which codes for a specific antibody or antigen-binding portion of the Invention as defined above. The skilled person will be able to determine substitutions, deletions and/or additions to such nucleic acids which will still provide an antibody or antigen-binding portion thereof in accordance with the present invention.

The present invention also provides constructs in the form of plasmids, vectors, transcription or expression cassettes which comprise at least one nucleic acid as described above. The present invention also provides a recombinant host cell which comprises one or more constructs as above. As mentioned, a nucleic acid encoding an antibody or antigen-binding portion of the invention forms an aspect of the present invention, as does a method of production of such, which method comprises expression from encoding nucleic acid therefor. Expression may conveniently be achieved by culturing under appropriate conditions recombinant host cells containing the nucleic acid. Following production by expression, a specific monoclonal antibody may be isolated and/or purified using any suitable technique, then used as appropriate.

Systems for cloning and expression of a polypeptide in a variety of different host cells are well known. Suitable host cells include bacteria, mammalian cells, yeast and insect cell (baculovirus) systems. Mammalian cell lines available in the art for expression of a heterologous polypeptide include Chinese hamster ovary cells, Hela cells, baby hamster kidney cells, NSO mouse melanoma cells and many others. Expression in eukaryotic cells in culture is available to those skilled in the art as an option for production of a specific monoclonal antibody, see for example Frenzel A, Hust M, Schirrmann T. Expression of recombinant antibodies. Front Immunol. 2013 Jul. 29; 4:217. doi: 10.3389/fimmu.2013.00217. PMID: 23908655; PMCID: PMC3725456.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral, e.g. phage, or phagemid, as appropriate. For further details see, for example, Sambrook et al., (1989). Many known techniques and protocols for manipulation of nucleic acid, for example in preparation of nucleic acid constructs, mutagenesis, sequencing, introduction of DNA into cells and gene expression, and analysis of proteins, are described in detail in Ausubel et al., (1992).

Therefore, according to another aspect of the present invention, there is provided a cell comprising the aforementioned nucleic acid. In one embodiment, the cell is a mammalian cell, such as a CHO or HEK cell. The antibody or antigen-binding fragment thereof may be recombinantly expressed in any of the cells selected from CHO, NSO, Sp2/0, HEK293, and PER.C6.

A further aspect of the present invention provides a host cell containing nucleic acid as disclosed herein. In one embodiment, the nucleic acid of the invention is integrated into the genome (e.g. chromosome) of the host cell. Integration may be promoted by inclusion of sequences which promote recombination with the genome, in accordance with standard techniques.

In further aspects, the invention provides methods of preparing specific antibodies or antigen-binding fragments of the invention which comprise expressing said nucleic acids under conditions to bring about expression of said antibody or antigen-binding fragment in a cell, and recovering the antibody or antigen-binding fragment.

Specific antibodies or antigen-binding fragments according to the invention may be used in a method of treatment of the human or animal body, such as a method of treatment of ischemia in a patient (preferably human) which comprises administering to said patient an effective amount of a specific antibody or antigen-binding fragment of the Invention.

### BRIEF DESCRIPTION OF THE FIGURES

FIG. 1: PCR using several combinations of Ig variable domain primers.

FIG. 2: Sequence alignment of heavy chains using a Clustal W 2.1 multiple sequence alignment tool (www.expasy.ch).

FIG. 3: 2D representation or Collier de Perles of the V-REGION of the heavy chain CDR loops (Lefranc, M,-P, et al., Dev. Comp. Immunol., 27, 55-77 (2003) PMID: 12477501 Amino acids are shown in the one-letter abbreviation. The CDRs assigned using the IMGT numbering system are limited by amino acids shown in squares (anchor positions), which belong to the neighbouring FR-IMGT. Hatched circles correspond to missing positions according to the IMGT unique numbering, Arrows indicate the direction of the beta strands and their different in 3D structures (from IMGT repertoire, http://imgt.cines.fr).

FIG. 4: Sequence alignment of light chains using a Clustal W 2.1 multiple sequence alignment tool (www.expasy.ch).

FIG. 5: 2D representation or Collier de Perles of the V-REGION of the light chain CDR loops (Lefranc, M.-P. eta/., Dev, Comp. Immunol., 27, 55-77 (2003) PMID: 12477501). Amino acids are shown in the one-letter abbreviation. The CDRs assigned using the IMGT numbering system are limited by amino acids shown in squares (anchor positions), which belong to the neighbouring FR-IMGT. Hatched circles correspond to missing positions according to the IMGT unique numbering. Arrows indicate the direction of the beta strands and their t different designations=structures (from IMGT repertoire, http://imgt.cines.fr).

FIG. 6: 4-20% denaturing, reducing (A) and non-reducing (B), SDS-PAGE analysis of Ab 1126 HC0 LC0 to HC1 LC5 #250719. Molecular weight marker shown in kilodaltons. Lanes are as follows:

| Lane | | | Amount | |
|---|---|---|---|---|
| number | Sample | Lot | (μg) | Conditions* |
| 1 | PageRule (Thermo | NA | NA | R/NR |
| | Fisher) | | | |
| 2 | Ab 1126 HC0 LC0 | 250719 | 2 | R/NR |
| 3 | Ab 1126 HC1 LC1 | 250719 | 2 | R/NR |
| 4 | Ab 1126 HC1 LC2 | 250719 | 2 | R/NR |
| 5 | Ab 1126 HC1 LC3 | 250719 | 2 | R/NR |
| 6 | Ab 1126 HC1 LC4 | 250719 | 2 | R/NR |
| 7 | Ab 1126 HC1 LC5 | 250719 | 2 | R/NR |

*R = reducing; NR = non-reducing.

FIG. 7: 4-20% denaturing, reducing (A) and non-reducing (B), SDS-PAGE analysis of Ab 1126 HC2 LC1 to HC2 LC5 #250719 and #080819. Molecular weight marker shown in kilodaltons. Lanes are as follows:

| Lane | | | Amount | |
|---|---|---|---|---|
| number | Sample | Lot | (μg) | Conditions* |
| 1 | PageRule (Thermo | NA | NA | R/NR |
| | Fisher) | | | |
| 2 | Ab 1126 HC2 LC1 | 250719 | 2 | R/NR |
| 3 | Ab 1126 HC2 LC2 | 250719 | 2 | R/NR |
| 4 | Ab 1126 HC2 LC3 | 080819 | 2 | R/NR |
| 5 | Ab 1126 HC2 LC4 | 250719 | 2 | R/NR |
| 6 | Ab 1126 HC2 LC5 | 250719 | 2 | R/NR |

*R = reducing; NR = non-reducing.

FIG. 8: 4-20% denaturing, reducing (A) and non-reducing (B), SDS-PAGE analysis of Ab 1126 HC3 LC1 to HC3 LC5 #250719. Molecular weight marker shown in kilodaltons. Lanes are as follows:

| Lane | | | Amount | |
|---|---|---|---|---|
| number | Sample | Lot | (μg) | Conditions* |
| 1 | PageRule (Thermo | NA | NA | R/NR |
| | Fisher) | | | |
| 2 | Ab 1126 HC3 LC1 | 250719 | 2 | R/NR |
| 3 | Ab 1126 HC3 LC2 | 250719 | 2 | R/NR |
| 4 | Ab 1126 HC3 LC3 | 250719 | 2 | R/NR |
| 5 | Ab 1126 HC3 LC4 | 250719 | 2 | R/NR |
| 6 | Ab 1126 HC3 LC5 | 250719 | 2 | R/NR |

*R = reducing; NR = non-reducing.

FIG. 9: 4-20% denaturing, reducing (A) and non-reducing (B), SDS-PAGE analysis of Ab 1126 HC4 LC1 to HC4 LC5 #250719. Molecular weight marker shown in kilodaltons. Lanes are as follows:

| Lane | | | Amount | |
|---|---|---|---|---|
| number | Sample | Lot | (μg) | Conditions* |
| 1 | PageRule (Thermo | NA | NA | R/NR |
| | Fisher) | | | |
| 2 | Ab 1126 HC4 LC1 | 250719 | 2 | R/NR |
| 3 | Ab 1126 HC4 LC2 | 250719 | 2 | R/NR |
| 4 | Ab 1126 HC4 LC3 | 250719 | 2 | R/NR |
| 5 | Ab 1126 HC4 LC4 | 250719 | 2 | R/NR |
| 6 | Ab 1126 HC4 LC5 | 250719 | 2 | R/NR |

*R = reducing; NR = non-reducing.

FIG. 10: 4-20% denaturing, reducing (A) and non-reducing (B), SDS-PAGE analysis of Ab 1126 HC5 LC1 to HC5 LC5 #250719. Molecular weight marker shown in kilodaltons. Lanes are as follows:

| Lane | | | Amount | |
|---|---|---|---|---|
| number | Sample | Lot | (μg) | Conditions* |
| 1 | PageRule (Thermo | NA | NA | R/NR |
| | Fisher) | | | |
| 2 | Ab 1126 HC5 LC1 | 250719 | 2 | R/NR |
| 3 | Ab 1126 HC5 LC2 | 250719 | 2 | R/NR |
| 4 | Ab 1126 HC5 LC3 | 250719 | 2 | R/NR |
| 5 | Ab 1126 HC5 LC4 | 250719 | 2 | R/NR |
| 6 | Ab 1126 HC5 LC5 | 250719 | 2 | R/NR |

*R = reducing; NR = non-reducing.

FIG. 11: Characterization of IgG: antigen interactions. Reference corrected BLI binding curves (black), monitored on a surface of noncovalently immobilized antibody (variant ID shown below sensorgram) for various antigen concentrations at 25 °C in running buffer. The apparent dissociation rate constant (ka) and association rate (Kₐ) constants were determined by globally fitting a 1:1 binding model to the sensorgrams using the software supplied with the instrument. Global fitting results are summarized in Table 2.

FIG. 12: 4-20% denaturing, reducing and non-reducing, SDS-PAGE analysis of Ab 1126 HC0 LC0 lot #011020. Molecular weight marker shown in kilodaltons. Lanes are as follows:

| Lane | | | Amount | |
|---|---|---|---|---|
| number | Sample | Lot | (μg) | Conditions |
| 1 | PageRule (Thermo | NA | NA | Reducing |
| | Fisher) | | | |
| 2 | Ab 1126 HC0 LC0 | 011020 | 2 | Reducing |
| 3 | Blank | NA | NA | NA |
| 4 | Ab 1126 HC0 LC0 | 011020 | 2 | Non-reducing |

FIG. 13: 4-20% denaturing, reducing and non-reducing, SDS-PAGE analysis of Ab 1126 HC4 LC2 lot #061020. Molecular weight marker shown in kilodaltons. Lanes are as follows:

| Lane | | | Amount | |
|---|---|---|---|---|
| number | Sample | Lot | (μg) | Conditions |
| 1 | PageRule (Thermo | NA | NA | Reducing |
| | Fisher) | | | |
| 2 | Ab 1126 HC4 LC2 | 061020 | 2 | Reducing |
| 3 | Blank | NA | NA | NA |
| 4 | Ab 1126 HC4 LC2 | 061020 | 2 | Non-reducing |

FIG. 14: 4-20% denaturing, reducing and non-reducing, SDS-PAGE analysis of Ab 1126 HC5 LC2 lot #061020. Molecular weight marker shown in kilodaltons. Lanes are as follows:

| Lane | | | Amount | |
|---|---|---|---|---|
| number | Sample | Lot | (μg) | Conditions |
| 1 | PageRule (Thermo | NA | NA | Reducing |
| | Fisher) | | | |
| 2 | Ab 1126 HC5 LC2 | 061020 | 2 | Reducing |
| 3 | Blank | NA | NA | NA |
| 4 | Ab 1126 HC5 LC2 | 061020 | 2 | Non-reducing |

FIG. 15: Analytical gel-filtration elution profile observed for Ab 1126 HC0 LC0. The sample Ab 1126 HC0 LC0 lot 011020 was analysed, and a chromatogram was obtained. The Chromatogram is shown, and the integration results are shown in corresponding table. The peak corresponding to the monomeric fraction (based on the retention volume of control proteins [see FIGS. 18 and 19]) is >98%. SEC analysis of Ab 1126 variant HC0 LC0 using a Superdex 200 Increase 10/300 GL column (see FIG. 18). Acetone control samples were run before and after samples and were within experimental error, identical (see FIG. 19),

FIG. 16: Analytical gel-filtration elution profile observed for Ab 1126 HC4 LC2. The sample Ab 1126 HC4 LC2 lot 061020 was analysed, and a chromatogram was obtained. The Chromatogram is shown, and the integration results are shown in corresponding table. The peak corresponding to the monomeric fraction (based on the retention volume of control proteins [see FIGS. 18 and 19]) is >98%. SEC analysis of Ab 1126 variant HC4 LC2 using a Superdex 200 Increase 10/300 GL column (see FIG. 18). Acetone control samples were run before and after samples and were within experimental error, identical (see FIG. 19).

FIG. 17: Analytical gel-filtration elution profile observed for Ab 1126 HC5 LC2. The sample Ab 1126 HC5 LC2 lot 061020 was analysed, and a chromatogram was obtained. The Chromatogram is shown, and the integration results are shown in corresponding table. The peak corresponding to the monomeric fraction (based on the retention volume of control proteins [see FIGS. 18 and 19]) is >98%, SEC analysis of Ab 1126 variant HC5 LC2 using a Superdex 200 Increase 10/300 GL column (see FIG. 18). Acetone control samples were run before and after samples and were within experimental error, identical (see FIG. 19).

FIG. 18: HMW calibration kit. Calibration data for Superdex 200 Increase 10/300 GL.

| | | Stokes | | | |
|---|---|---|---|---|---|
| | Approx. | Radius | Retention | Area | |
| Peak | MW (Da) | (nm) | (mL) | (mL*mAU) | Area % |
| Ferritin | 880,000 | — | 8.47 | 11.17 | 8.58 |
| (dimer) | | | | | |
| Thyroglobulin | 669,000 | 8.6 | 8.98 | 22.01 | 16.9 |
| Ferritin | 440,000 | 6.1 | 10.13 | 7.41 | 5.68 |
| Aldolase | 158,000 | 4.8 | 12.28 | 31.93 | 24.52 |
| Conalbumin | 75,000 | 3.5 | 13.76 | 32.63 | 25.05 |
| Ovalbumin | 43,000 | 2.8 | 14.68 | 25.10 | 19.27 |
| Typical IgG | 158,000 | 5.2 | 11.8-12.3 | — | — |

FIG. 19: SEC analysis of acetone control using a Superdex 200 Increase 10/300 GL column. SEC analysis for acetone control samples, performed before and after sample analysis.

FIG. 20: Model of Fv domain of humanized VH1/VL1 predicted by Abodybuilder. Potential sequence liabilities are circled (N-glycosylation; Met oxidation; Trp oxidation; Asp isomerization). The same potential sequence liabilities are present in all of the humanized variants.

FIG. 21: (A) A pictographic portrayal of anti-VEGF-A, anti-VEGF₁₆₅b, VEGF₁₆₅b and rh-Fc-VEGFR2. (B) The inhibitory effect of anti-VEGF: 65b (HC4 LC2) on VEGF₁₆₅b binding to fc-VEGF receptor 2.

FIG. 22: Cell migration assay for chimeric anti-VEGF₁₆₅b (HC0 LC0) and humanised anti-VEGF₁₆₅b (HC4 LC2 Vexobicizumab) and mouse anti-VEGF₁₆₅b antibody (mx165b) in the presence of VEGF₁₆₅a.

FIG. 23: Cell migration assay for mα165b, chimeric anti-VEGF₁₆₅b (HC0 LC0) and humanised anti-VEGF₁₆₅b (HC4 LC2 Vexobicizumab) and previously published mouse anti-VEGF₁₆₅b antibody (MRVL56/1, AbCam) in the presence of VEGF₁₆₅a. B. IC50 for each of the antibodies (Vex=HC4 LC2).

FIG. 24: (A) Pictographic representation of a cell migration assay in diabetic PAD human monocyte mediated inhibition of endothelial migration. (B) Results of the cell migration assay,

FIG. 25: A schematic of the mouse experiment described in Example 4.

FIG. 26: Murine analysis of revascularisation in hind limb ischemia. Examples of mice fed a high fat high sucrose (HFHS) diet for 12 weeks followed by blood flow imaging by laser speckle of mice before (Pre-Op) and after (Post-Op) femoral artery ligation, and on subsequent days.

FIG. 27: Graphical quantification of the murine analysis of blood flow in hind limb ischemia. Speckle intensity was calculated as the ipsilateral (ischemic) relative to the contralateral flow. Blood flow relative to the ratio pre-op. **=p<0.05 compared with IgG on the same day, Two-way ANOVA.

FIG. 28: (A) Examples of mouse gastrocnemius muscle 28 days after ischemia and treatment with either IgG of Vexobicizumab; (B) Angiogenesis was calculated as the ipsilateral (ischemic) relative to the contralateral IB4+ve vessels; (C) Arteriogenesis was calculated as the ipsilateral (ischemic) relative to the contralateral smooth muscle actin (SMA)+ve vessels. **=p<0.01, ***=p<0.001 compared with IgG, Unpaired t test.

FIG. 29: A schematic of the rat experiment described in Example 4.

FIG. 30: Rat analysis of revascularisation in hind limb ischemia. Examples of paw blood flow imaging by laser speckle of rats before (Pre-Op) and after (Post-Op) femoral artery ligation, and on subsequent days. Speckle intensity was calculated as the ipsilateral (ischemic) relative to contralateral flow.

FIG. 31: Graphical quantification of the rat analysis of blood flow in hind limb ischemia, *=p<0.05 compared with IgG on the same day, #=p<0.05 versus pre-op, ###=P<0.001 versus pre-op, Two way ANOVA.

FIG. 32: (A) Examples of STZ rat gastrocnemius muscle 28 days after ischemia and treatment with either IgG of Vexobicizumab; (B) Angiogenesis was calculated as the ipsilateral (ischemic) relative to the contralateral IB4+ve vessels; (C) Arteriogenesis was calculated as the ipsilateral (ischemic) relative to the contralateral smooth muscle actin (SMA)+ve vessels. **=p<0.01, ***=p<0.001 compared with IgG, Unpaired t test.

FIG. 33: A. Human monocytes inhibit migration of endothelial cells across a porous membrane. 1 μg/ml HC4 LC2 significantly reverses this inhibitory effect on migration of human umbilical vein cells, N=3, cells from a single subject. One way ANOVA, B. Monocytes from 7 patients with peripheral vascular disease inhibit migration towards VEGF-A₁₆₅a. C-D. Migration is increased by 1 μg/ml and 5 μg/ml HC4LC2. Paired t tests. *=p<0.05, **=p<0.01

FIG. 34: A single gene can result in opposing isoforms of VEGF-proangiogenic VEGF-A₁₆₅a and antiangiogenic VEGF-A₁₆₅b.

FIG. 35: A key to show the antibody derivation from parental hybridomas.

FIG. 36: To determine the neutralizing effect of the antibodies, VEGFR2 was bound to an ELISA plate and the anti-VEGF-A₁₆₅b antibodies mixed with rhVEGF-A₁₆₅b at increasing concentrations of VEGF-A₁₆₅b antibody. Humanised (HC4LC2) and chimeric (HC0LC0) anti-VEGF₁₆₅b were both able to significantly reduce the affinity of VEGF₁₆₅b to VEGF receptor 2 (VEGFR2), again, to a slightly lesser extent than the positive control of G6-31 (a pan VEGF antibody that binds to the receptor binding site of VEGF-A).

FIG. 37: Cell migration assay for humanised anti-VEGF₁₆₅b (HC5 LC2) and mouse anti-VEGF₁₆₅b antibody (mα165b-referred to as “56/8” in the figure) in the presence of VEGF₁₆₅a.

### EXAMPLES

### Example 1: Monoclonal Sequencing

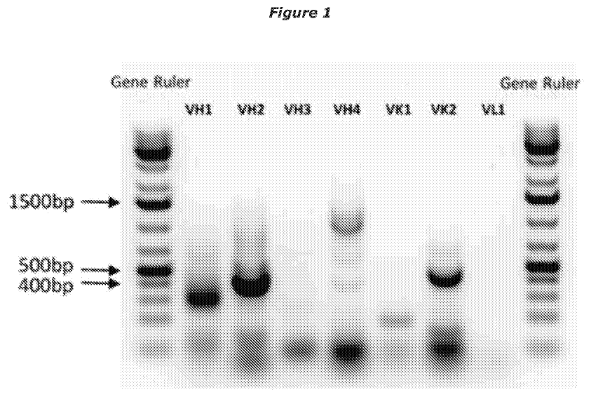

mRNA was extracted from hybridoma cell pellets. Total RNA was extracted from the pellets using Fusion Antibodies Pie in-house RNA extraction protocol, CDNA was reverse transcribed from the RNA with an oligo (dT) primer. PCR reactions were set up using variable domain primers to amplify both the VH and VL regions of the monoclonal antibody DNA. Agarose gel electrophoresis of the amplified PCR products are detailed in FIG. 1.

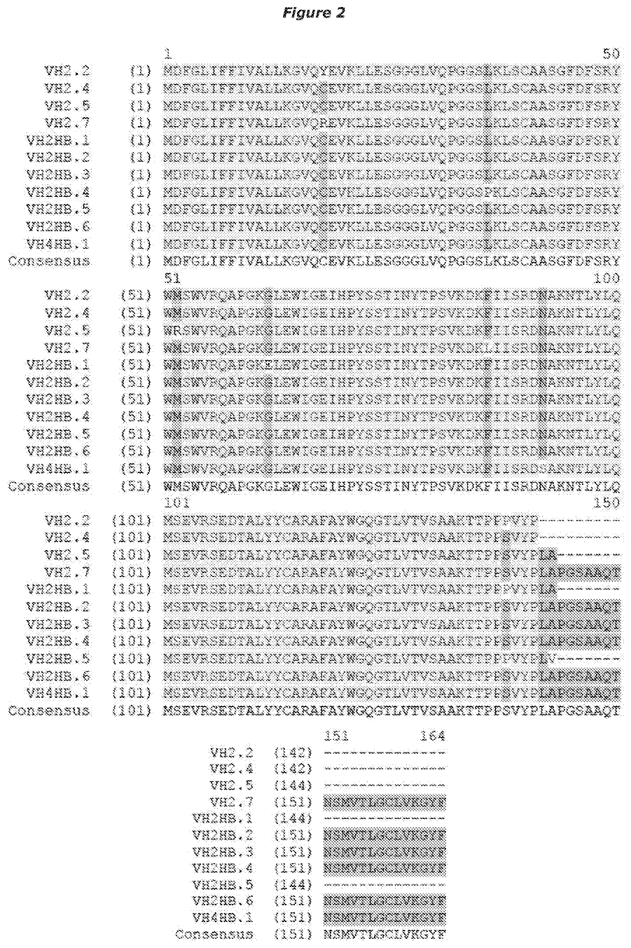

The VH and VL products were cloned into the Thermo Fisher sequencing vector pCR™2.1. TOP 10 cells were transformed with the cloned plasmid and screened by PCR for positive transformants. Selected colonies were picked and analysed by DNA sequencing on an ABI3130xI Genetic Analyzer, the result may be seen in FIG. 2, the sequences of which are reproduced below.

Heavy chain
VH2.2 protein
(SEQ ID NO: 38)
EVKLLESGGGLVQPGGSLKLSCAASGFDFSRYWMSWVRQAPGKGLEWIGEIHPYSSTINYTPSVKDKFIISR
DNAKNTLYLQMSEVRSEDTALYYCARAFAYWGQGTLVTVSAAKTTPPPVYP
VH2.2 nucleic acid
(SEQ ID NO: 39)
GAGGTGAAGCTTCTCGAATCTGGAGGTGGCCTGGTGCAGCCTGGAGGATCCCTGAAACTCTCCTGTGCAGCC
TCAGGATTCGATTTTAGTAGATACTGGATGAGTTGGGTCCGGCAGGCTCCAGGGAAAGGGCTAGAATGGATT
GGAGAAATTCATCCATATAGCAGTACGATAAACTATACGCCATCTGTAAAGGATAAATTCATCATCTCCAGA
GACAACGCCAAAAATACGCTGTACCTGCAAATGAGCGAAGTGAGATCTGAGGACACAGCCCTTTATTACTGT
GCAAGGGCGTTTGCTTACTGGGGCCAAGGGACTCTGGTCACTGTCTCTGCAGCCAAAACGACACCCCCACCC
GTTTATCCT
VH2.4 protein
(SEQ ID NO: 40)
EVKLLESGGGLVQPGGSLKLSCAASGFDFSRYWMSWVRQAPGKGLEWIGEIHPYSSTINYTPSVKDKFIISR
DNAKNTLYLQMSEVRSEDTALYYCARAFAYWGQGTLVTVSAAKTIPPSVYP
VH2.4 nucleic acid
(SEQ ID NO: 41)
GAGGTGAAGCTTCTCGAATCTGGAGGTGGCCTGGTGCAGCCTGGAGGATCCCTGAAACTCTCCTGTGCAGCC
TCAGGATTCGATTTTAGTAGATACTGGATGAGTTGGGTCCGGCAGGCTCCAGGGAAAGGGCTAGAATGGATT
GGAGAAATTCATCCATATAGCAGTACGATAAACTATACGCCATCTGTAAAGGATAAATTCATCATCTCCAGA
GACAACGCCAAAAATACGCTGTACCTGCAAATGAGCGAAGTGAGATCTGAGGACACAGCCCTTTATTACTGT
GCAAGGGCGTTTGCTTACTGGGGCCAAGGGACTCTGGTCACTGTCTCTGCAGCCAAAACGACACCCCCATCC
GTCTATCCC
VH2.5 protein
(SEQ ID NO: 42)
EVKLLESGGGLVQPGGSLKLSCAASGFDFSRYWRSWVRQAPGKGLEWIGEIHPYSSTINYTPSVKDKFIISR
DNAKNTLYLQMSEVRSEDTALYYCARAFAYWGQGTLVTVSAAKTTPPSVYPLA
VH2.5 nucleic acid
(SEQ ID NO: 43)
GAGGTGAAGCTTCTCGAATCTGGAGGTGGCCTGGTGCAGCCTGGAGGATCCCTGAAACTCTCCTGTGCAGCC
TCAGGATTCGATTTTAGTAGATACTGGAGGAGTTGGGTCCGGCAGGCTCCAGGGAAAGGGCTAGAATGGATT
GGAGAAATTCATCCATATAGTAGTACGATAAACTATACGCCATCTGTAAAGGATAAATTCATCATCTCCAGA
GACAACGCCAAAAATACGCTGTACCTGCAAATGAGCGAAGTGAGATCTGAGGACACAGCCCTTTATTACTGT
GCAAGGGCGTTTGCTTACTGGGGCCAAGGGACTCTGGTCACTGTCTCTGCAGCCAAAACGACACCCCCATCC
GTCTATCCCTTGGCC
VH2.7 protein
(SEQ ID NO: 44)
EVKLLESGGGLVQPGGSLKLSCAASGFDFSRYWMSWVRQAPGKGLEWIGEIHPYSSTINYTPSVKDKLIISR
DNAKNTLYLQMSEVRSEDTALYYCARAFAYWGQGTLVTVSAAKTTPPSVYPLAPGSAAQTNSMVTLGCLVKG
YF
VH2.7 nucleic acid
(SEQ ID NO: 45)
GAGGTGAAGCTTCTCGAATCTGGAGGTGGTCTGGTGCAGCCTGGAGGATCCCTGAAACTCTCCTGTGCAGCC
TCAGGATTCGATTTTAGTAGATACTGGATGAGTTGGGTCCGGCAGGCTCCAGGGAAAGGGCTAGAATGGATT
GGAGAAATTCATCCATATAGCAGTACGATAAACTATACGCCATCTGTAAAGGATAAATTAATCATCTCCAGA
GACAACGCCAAAAATACGCTGTACCTGCAAATGAGCGAAGTGAGATCTGAGGACACAGCCCTTTATTACTGT
GCAAGGGCGTTTGCTTACTGGGGCCAAGGGACTCTGGTCACTGTCTCTGCAGCCAAAACGACACCCCCATCT
GTCTATCCACTGGCCCCTGGATCTGCTGCCCAAACTAACTCCATGGTGACCCTGGGATGCCTTGTCAAAGGT
TATTTC
VH2HB.1 protein
(SEQ ID NO: 46)
EVKLLESGGGLVQPGGSLKLSCAASGFDFSRYWMSWVRQAPGKELEWIGEIHPYSSTINYTPSVKDKFIISR
DNAKNTLYLQMSEVRSEDTALYYCARAFAYWGQGTLVTVSAAKTTPPPVYPLA
VH2HB. 1 nucleic acid
(SEQ ID NO: 47)
GAGGTGAAGCTTCTCGAATCTGGAGGTGGCCTGGTGCAGCCTGGAGGATCCCTGAAACTCTCCTGTGCAGCC
TCAGGATTCGATTTTAGTAGATACTGGATGAGTTGGGTCCGGCAGGCTCCAGGGAAAGAGCTAGAATGGATT
GGAGAAATTCATCCATATAGCAGTACGATAAACTATACGCCATCTGTAAAGGATAAATTCATCATCTCCAGA
GACAACGCCAAAAATACGCTGTACCTGCAAATGAGCGAAGTGAGATCTGAGGACACAGCCCTTTATTACTGT
GCAAGGGCGTTTGCTTACTGGGGCCAAGGGACTCTGGTCACTGTCTCTGCAGCCAAAACGACACCCCCACCC
GTTTATCCATTGGCC
VH2HB.2 protein
(SEQ ID NO: 48)
EVKLLESGGGLVQPGGSLKLSCAASGEDESRYWMSWVRQAPGKGLEWIGEIHPYSSTINYTPSVKDKFIISR
DNAKNTLYLQMSEVRSEDTALYYCARAFAYWGQGTLVTVSAAKTTPPSVYPLAPGSAAQTNSMVTLGCLVKG
YF
VH2HB.2 nucleic acid
(SEQ ID NO: 49)
GAGGTGAAGCTTCTCGAATCTGGAGGTGGCCTGGTGCAGCCTGGAGGATCCCTGAAACTCTCCTGTGCAGCC
TCAGGATTCGATTTTAGTAGATACTGGATGAGTTGGGTCCGGCAGGCTCCAGGGAAAGGGCTAGAATGGATT
GGAGAAATTCATCCATATAGCAGTACGATAAACTATACGCCATCCGTAAAGGATAAATTCATCATCTCCAGA
GACAACGCCAAAAATACGCTGTACCTGCAAATGAGCGAAGTGAGATCTGAGGACACAGCCCTTTATTACTGT
GCAAGGGCGTTTGCTTACTGGGGCCAAGGGACTCTGGTCACTGTCTCTGCAGCCAAAACGACACCCCCATCT
GTCTATCCACTGGCCCCTGGATCTGCTGCCCAAACTAACTCCATGGTGACCCTGGGATGCCTGGTCAAAGGT
TATTTC
VH2HB.3 protein
(SEQ ID NO: 48)
EVKLLESGGGLVQPGGSLKLSCAASGFDFSRYWMSWVRQAPGKGLEWIGEIHPYSSTINYTPSVKDKFIISR
DNAKNTLYLQMSEVRSEDTALYYCARAFAYWGQGTLVTVSAAKTTPPSVYPLAPGSAAQTNSMVTLGCLVKG
YF
VH2HB.3 nucleic acid
(SEQ ID NO: 50)
GAGGTGAAGCTTCTCGAATCTGGAGGTGGCCTGGTGCAGCCTGGAGGATCCCTGAAACTCTCCTGTGCAGCC
TCAGGATTCGATTTTAGTAGATACTGGATGAGTTGGGTCCGGCAGGCTCCAGGGAAAGGGCTAGAATGGATT
GGAGAAATTCATCCATATAGCAGTACGATAAACTATACGCCATCTGTAAAGGATAAATTCATCATCTCCAGA
GACAACGCCAAAAATACGCTGTACCTGCAAATGAGCGAAGTGAGATCTGAGGACACAGCCCTTTATTACTGT
GCAAGGGCGTTTGCTTACTGGGGCCAAGGGACTCTGGTCACTGTCTCTGCAGCCAAAACGACACCCCCATCT
GTCTATCCACTGGCCCCTGGATCTGCTGCCCAAACTAACTCCATGGTGACCCTGGGATGCCTTGTCAAAGGT
TATTTC
VH2HB.4 protein
(SEQ ID NO: 51)
EVKLLESGGGLVQPGGSPKLSCAASGFDFSRYWMSWVRQAPGKGLEWIGEIHPYSSTINYTPSVKDKFIISR
DNAKNTLYLQMSEVRSEDTALYYCARAFAYWGQGTLVTVSAAKTTPPSVYPLAPGSAAQTNSMVTLGCLVKG
YF
VH2HB.4 nucleic acid
(SEQ ID NO: 52)
GAGGTGAAGCTTCTCGAATCTGGAGGTGGCCTGGTGCAGCCTGGAGGATCCCCGAAACTCTCCTGTGCAGCC
TCAGGATTCGATTTTAGTAGATACTGGATGAGTTGGGTCCGGCAGGCTCCAGGGAAAGGGCTAGAATGGATT
GGAGAAATTCATCCATATAGCAGTACGATAAACTATACGCCATCTGTAAAGGATAAATTCATCATCTCCAGA
GACAACGCCAAAAATACGCTGTACCTGCAAATGAGCGAAGTGAGATCTGAGGACACAGCCCTTTATTACTGT
GCAAGGGCGTTTGCTTACTGGGGCCAAGGGACTCTGGTCACTGTCTCTGCAGCCAAAACGACACCCCCATCC
GTCTATCCACTGGCCCCTGGATCTGCTGCCCAAACTAACTCCATGGTGACCCTGGGATGCCTGGTCAAAGGT
TATTTC
VH2HB.5 protein
(SEQ ID NO: 53)
EVKLLESGGGLVQPGGSLKLSCAASGFDFSRYWMSWVRQAPGKGLEWIGEIHPYSSTINYTPSVKDKFIISR
DNAKNTLYLQMSEVRSEDTALYYCARAFAYWGQGTLVTVSAAKTTPPPVYPLV
VH2HB.5 nucleic acid
(SEQ ID NO: 54)
GAGGTGAAGCTTCTCGAATCTGGAGGTGGCCTGGTGCAGCCTGGAGGATCCCTGAAACTCTCCTGTGCAGCC
TCAGGATTCGATTTTAGTAGATACTGGATGAGTTGGGTCCGGCAGGCTCCAGGGAAAGGGCTAGAATGGATT
GGAGAAATTCATCCATATAGCAGTACGATAAACTATACGCCATCTGTAAAGGATAAATTCATCATCTCCAGA
GACAACGCCAAAAATACGCTGTACCTGCAAATGAGCGAAGTGAGATCTGAGGACACAGCCCTTTATTACTGT
GCAAGGGCGTTTGCTTACTGGGGCCAAGGGACTCTGGTCACTGTCTCTGCAGCCAAAACGACACCCCCACCC
GTCTATCCCTTGGTC
VH2HB.6 protein
(SEQ ID NO: 48)
EVKLLESGGGLVQPGGSLKLSCAASGEDESRYWMSWVRQAPGKGLEWIGEIHPYSSTINYTPSVKDKFIISR
DNAKNTLYLQMSEVRSEDTALYYCARAFAYWGQGTLVTVSAAKTTPPSVYPLAPGSAAQTNSMVTLGCLVKG
YF
VH2HB.6 nucleic acid
(SEQ ID NO: 55)
GAGGTGAAGCTTCTCGAATCTGGAGGTGGCCTGGTGCAGCCTGGAGGATCCCTGAAACTCTCCTGTGCAGCC
TCAGGATTCGATTTTAGTAGATACTGGATGAGTTGGGTCCGGCAGGCTCCAGGGAAAGGGCTAGAATGGATT
GGAGAAATTCATCCATATAGCAGTACGATAAACTATACGCCATCTGTAAAGGATAAATTCATCATCTCCAGA
GACAACGCCAAAAATACGCTGTACCTGCAAATGAGCGAAGTGAGATCTGAGGACACAGCCCTTTATTACTGT
GCAAGGGCGTTTGCTTACTGGGGCCAAGGGACTCTGGTCACTGTCTCTGCAGCCAAAACGACACCCCCATCT
GTCTATCCACTGGCCCCTGGATCTGCTGCCCAAACTAACTCCATGGTGACCCTGGGATGCCTGGTCAAGGGT
TATTTC
VH4HB.1 protein
(SEQ ID NO: 56)
EVKLLESGGGLVQPGGSLKLSCAASGFDFSRYWMSWVRQAPGKGLEWIGEIHPYSSTINYTPSVKDKFIISR
DSAKNTLYLQMSEVRSEDTALYYCARAFAYWGQGTLVTVSAAKTTPPSVYPLAPGSAAQTNSMVTLGCLVKG
YF
VH4HB. 1 nucleic acid
(SEQ ID NO: 57)
GAGGTGAAGCTTCTCGAATCTGGAGGTGGCCTGGTGCAGCCTGGAGGATCCCTGAAACTCTCCTGTGCAGCC
TCAGGATTCGATTTTAGTAGATACTGGATGAGTTGGGTCCGGCAGGCTCCAGGGAAAGGGCTAGAATGGATT
GGAGAAATTCATCCATATAGCAGTACGATAAACTATACGCCATCTGTAAAGGATAAATTCATCATCTCCAGA
GACAGCGCCAAAAATACGCTGTACCTGCAAATGAGCGAAGTGAGATCTGAGGACACAGCCCTTTATTACTGT
GCAAGGGCGTTTGCTTACTGGGGCCAAGGGACTCTGGTCACTGTCTCTGCAGCCAAAACGACACCCCCATCT
GTCTATCCACTGGCCCCTGGATCTGCTGCCCAAACTAACTCCATGGTGACCCTGGGATGCCTGGTCAAGGGC
TATTTC

Consensus (SEQ ID NO: 58) (positions in parenthesis indicate potential substitutions at that position, and X=blank)

EVKLLESGGGLVQPGGS(L/P)KLSCAASGEDESRY
W(M/R)SWVRQAPGK(G/E)LEWIGEIHPYSSTINY
TPSVKDK(F/L)IISRD(N/S)AKNTLYLQMSEVRS
EDTALYYCARAFAYWGQGTLVTVSAAKTTPP(S/P)
VYP(L/X)(A/V/X)(P/X)(G/X)(S/X)(A/X)
(A/X)(Q/X)(T/X)(N/X)(S/X)(M/X)(V/X)
(T/X)(L/X)(G/X)(C/X)(L/X)(V/X)(K/X)
(G/X)(Y/X)(F/X)

Consensus sequence in which the most common amino acids are indicated at each position (SEQ ID NO: 48) (variable domain is highlighted in bold, with sequencing primers underlined, and CDRs double underlined)

EVKLLESGGGLVQPGGSLKLSCAASGFDFSRYWMSW
VRQAPGKGLEWIGEIHPYSSTINYTPSVKDKFIISR
DNAKNTLYLQMSEVRSEDTALYYCARAFAYWGQGTL
VTVSAAKTTPPSVYPLAPGSAAQTNSMVTLGCLVKG
YF

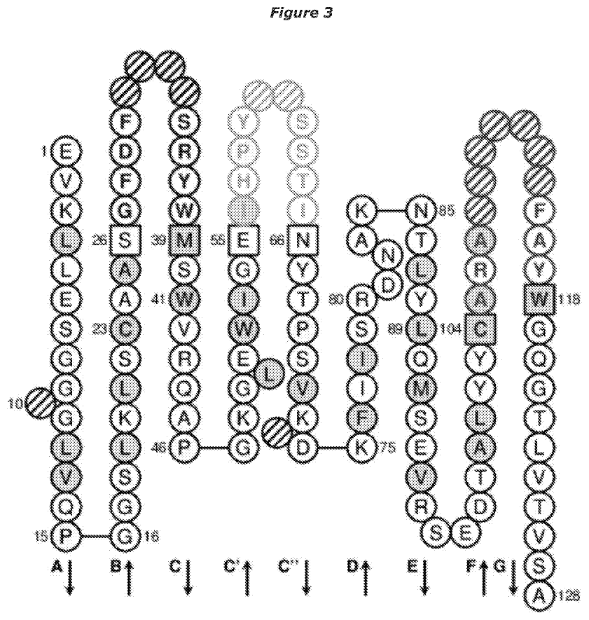

The complementarity determining regions (CDRs) indicated by double underlining were determined by the IMGT numbering system (Lefranc, M.-P, et al., Nucleic Acids Research, 27, 209-272 (1999)). The consensus amino acid sequence from VH sequencing was obtained from at least 5 amino acid sequences as shown by the alignment in FIG. 2. A graphical representation of the variable domain with IMGT labelling is shown in FIG. 3.

Light chain
VL2.1 protein
(SEQ ID NO: 137)
DIVMTQTPLTLSVTIGQPASISCKSSQSLLDSDGKTYLNWLLQRPGQSPKRLIYLVSKLDSGVPDRFTGSGS
GTDFTLKISRVEAEDLGVYYCWQGTHEPYTFGGGTKVEIKRADAAPTVSIFPPSSEQLTSGGASVVCFLNNF
VL2.1 nucleic acid
(SEQ ID NO: 149)
GATATTGTGATGACCCAGACTCCACTCACTTTGTCGGTTACCATTGGACAGCCAGCCTCCATCTCTTGCAAG
TCAAGTCAGAGCCTCTTAGATAGTGATGGAAAGACATATTTGAATTGGTTGTTACAGAGGCCAGGCCAGTCT
CCAAAGCGCCTAATCTATCTGGTGTCTAAACTGGACTCTGGAGTCCCTGACAGGTTCACTGGCAGTGGATCA
GGGACAGATTTCACACTGAAAATCAGCAGAGTGGAGGCTGAGGATTTGGGAGTTTATTATTGCTGGCAAGGT
ACACATTTTCCGTACACGTTCGGAGGGGGGACCAAGGTGGAAATAAAACGGGCTGATGCTGCACCAACTGTA
TCCATCTTCCCACCATCCAGTGAGCAGTTAACATCTGGAGGTGCCTCAGTCGTGTGCTTCTTGAACAACTTC
T
VL2.2 protein
(SEQ ID NO: 138)
DIVMTQTPLTLSVTIGQPASISCKSSQSLLDSDGKTYLNWLLQRPGQSPKRLIYLVSKLDSGVPDRFTGSGS
GTDFTLKISRVEAEDLGVYYCWQGTHEPYTFGGGTKVEIKRADAAPTVSIFPPSSEQLTSGGASVVC
VL2.2 nucleic acid
(SEQ ID NO: 150)
GATATTGTGATGACCCAGACTCCACTCACTTTGTCGGTTACCATTGGACAGCCAGCCTCCATCTCTTGCAAG
TCAAGTCAGAGCCTCTTAGATAGTGATGGAAAGACATATTTGAATTGGTTGTTACAGAGGCCAGGCCAGTCT
CCAAAGCGCCTAATCTATCTGGTGTCTAAACTGGACTCTGGAGTCCCTGACAGGTTCACTGGCAGTGGATCA
GGGACAGATTTCACACTGAAAATCAGCAGAGTGGAGGCTGAGGATTTGGGAGTTTATTATTGCTGGCAAGGT
ACACATTTTCCGTACACGTTCGGAGGGGGGACCAAGGTGGAAATAAAACGGGCTGATGCTGCACCAACTGTA
TCCATCTTCCCACCATCCAGTGAGCAGTTAACATCTGGAGGTGCCTCAGTCGTGTGC
VL2.3 protein
(SEQ ID NO: 139)
DIVMTQTPLTLSVTIGQPASISCKSSQSLLDSDGKTYLNWLLQRPGQSPKRLIYLVSKLDSGVPDRFTGSGS
GTDFTLKISRVEAEDLGVYYCWQGTHFPYTFGGGTKVEIKRADAAPTVSIFPPSSEQLTSGGASVVCFINNE
YPK
VL2.3 nucleic acid
(SEQ ID NO: 151)
GATATTGTGATGACCCAGACTCCACTCACTTTGTCGGTTACCATTGGACAGCCAGCCTCCATCTCTTGCAAG
TCAAGTCAGAGCCTCTTAGATAGTGATGGAAAGACATATTTGAATTGGTTGTTACAGAGGCCAGGCCAGTCT
CCAAAGCGCCTAATCTATCTGGTGTCTAAACTGGACTCTGGAGTCCCTGACAGGTTCACTGGCAGTGGATCA
GGGACAGATTTCACACTGAAAATCAGCAGAGTGGAGGCTGAGGATTTGGGAGTTTATTATTGCTGGCAAGGT
ACACATTTTCCGTACACGTTCGGAGGGGGGACCAAGGTGGAAATAAAACGGGCTGATGCTGCACCAACTGTA
TCCATCTTCCCACCATCCAGTGAGCAGTTAACATCCGGAGGTGCCTCAGTCGTGTGCTTCTTGAACAACTTC
TACCCCAAA
VL2.6 protein
(SEQ ID NO: 140)
DIVMTQTPLTLSVTIGQPASISCKSSQCLLDSDGKTYLNWLLQRPGQSPKRLIYLVSKLDSGVPDRFTGSGS
GTDFTLKISRVEAEDLGVYYCWQGTHEPYTFGGGTKVEIKRADAAPTVSIFPPSSEQLTSGGASVVCFINNE
YPR
VL2.6 nucleic acid
(SEQ ID NO: 152)
GATATTGTGATGACCCAGACTCCACTCACTTTGTCGGTTACCATTGGACAGCCAGCCTCCATCTCTTGCAAG
TCAAGTCAGTGCCTCTTAGATAGTGATGGAAAGACATATTTGAATTGGTTGTTACAGAGGCCAGGCCAGTCT
CCAAAGCGCCTAATCTATCTGGTGTCTAAACTGGACTCTGGAGTCCCTGACAGGTTCACTGGCAGTGGATCA
GGGACAGATTTCACGCTGAAAATCAGCAGAGTGGAGGCTGAGGATTTGGGAGTTTATTATTGCTGGCAAGGT
ACACATTTTCCGTACACGTTCGGAGGGGGGACCAAGGTGGAAATAAAACGGGCTGATGCTGCACCAACTGTA
TCCATCTTCCCACCATCCAGTGAGCAGTTAACATCTGGAGGTGCCTCAGTCGTGTGCTTCTTGAACAACTTC
TACCCCAGA
VL2.7 protein
(SEQ ID NO: 141)
DIVMTQTPLTLSVTIGQPASISCKSSQSLLDSDGKTYLNWLLQRPGQSPKRLIYLVSKLDSGVPDRFTGSGS
GTDFTLKISRVEAEDLGVYYCWQGTHEPYTFGGGTKVEIKRADAAPTVSIFPPSSEQLTSGGASVVCFLNNF
YPR
VL2.7 nucleic acid
(SEQ ID NO: 153)
GATATTGTGATGACCCAGACTCCACTCACTTTGTCGGTTACCATTGGACAGCCAGCCTCCATCTCTTGCAAG
TCAAGTCAGAGCCTCTTAGATAGTGATGGAAAGACATATTTGAATTGGTTGTTACAGAGGCCAGGCCAGTCT
CCAAAGCGCCTAATCTATCTGGTGTCTAAACTGGACTCTGGAGTCCCTGACAGGTTCACTGGCAGTGGATCA
GGGACAGATTTCACACTGAAAATCAGCAGAGTGGAGGCTGAGGATTTGGGAGTTTATTATTGCTGGCAAGGT
ACACATTTTCCGTACACGTTCGGAGGGGGGACCAAGGTGGAAATAAAACGGGCTGATGCTGCACCAACTGTA
TCCATCTTCCCACCATCCAGTGAGCAGTTAACATCTGGAGGTGCCTCAGTCGTGTGCTTCTTGAACAACTTC
TACCCCAGA
VL2.8 protein
(SEQ ID NO: 142)
DIVMTQTPLTLSVTIGQPASISCKSSQSLLDSDGKTYLNWLLQRPGQSPKRLIYLVSKLDSGVPDRFTGSGS
GTDFTLKISRVEAEDLGVYYCWQGTHFPYTFGGGTKVEIKRADAAPTVSIFPPSSEQLTSGGASVVCFINNE
YPR
VL2.8 nucleic acid
(SEQ ID NO: 154)
GATATTGTGATGACCCAGACTCCACTCACTTTGTCGGTTACCATTGGACAGCCAGCCTCCATCTCTTGCAAG
TCAAGTCAGAGCCTCTTAGATAGTGATGGAAAGACATATTTGAATTGGTTGTTACAGAGGCCAGGCCAGTCT
CCAAAGCGCCTAATCTATCTGGTGTCTAAACTGGACTCTGGAGTCCCTGACAGGTTCACTGGCAGTGGATCA
GGGACAGATTTCACACTGAAAATCAGCAGAGTGGAGGCTGAGGATTTGGGAGTTTATTATTGCTGGCAAGGT
ACACATTTTCCGTACACGTTCGGAGGGGGGACCAAGGTGGAAATAAAACGGGCTGATGCTGCACCAACTGTA
TCCATCTTCCCACCATCCAGTGAGCAGTTAACATCTGGAGGTGCCTCAGTCGTGTGCTTCTTGAACAACTTC
TACCCCAGA
VL2HB.2 protein
(SEQ ID NO: 143)
DIVMTQTPLTLSVTIGQPASISCKSSQSLLDSDGKTYLNWLLQRPGQSPKRLIYLVSKLDSGVPDRFTGSGS
GTDFTLKISRVEAEDLGVYYCWQGTHEPYTFGGGTKVEIKRADAAPTVSIFPPSSEQLTSGGASVVCFINNE
YPK
VL2HB.2 nucleic acid
(SEQ ID NO: 155)
GATATTGTGATGACCCAGACTCCACTCACTTTGTCGGTTACCATTGGACAGCCAGCCTCCATCTCTTGCAAG
TCAAGTCAGAGCCTCTTAGATAGTGATGGAAAGACATATTTGAATTGGTTGTTACAGAGGCCAGGCCAGTCT
CCAAAGCGCCTAATCTATCTGGTGTCTAAACTGGACTCTGGAGTCCCTGACAGGTTCACTGGCAGTGGATCA
GGGACAGATTTCACACTGAAAATCAGCAGAGTGGAGGCTGAGGATTTGGGAGTTTATTATTGCTGGCAAGGT
ACACATTTTCCGTACACGTTCGGAGGGGGGACCAAGGTGGAAATAAAACGGGCTGATGCTGCACCAACTGTA
TCCATCTTCCCACCATCCAGTGAGCAGTTAACATCTGGAGGTGCCTCAGTCGTGTGCTTCTTGAACAACTTC
TACCCCAAA
VL2HB.3 protein
(SEQ ID NO: 144)
DIVMTQTPLTLSVTIGQPASISCKSSQSLLDSDGKTYLNWLLQRPGQSPKRLIYLVSKLDSGVPDRFTGSGS
GTDFTLKISRVEAEDLGVYYCWQGTHEPYTFGGGTKVEIKRADAAPTVSIFPPSSEQLTSGGASVVCFLNNF
YPK
VL2HB.3 nucleic acid
(SEQ ID NO: 156)
GATATTGTGATGACCCAGACTCCACTCACTTTGTCGGTTACCATTGGACAGCCAGCCTCCATCTCTTGCAAG
TCAAGTCAGAGCCTCTTAGATAGTGATGGAAAGACATATTTGAATTGGTTGTTACAGAGGCCAGGCCAGTCT
CCAAAGCGCCTAATCTATCTGGTGTCTAAACTGGACTCTGGAGTCCCTGACAGGTTCACTGGCAGTGGATCA
GGGACAGATTTCACACTGAAAATCAGCAGAGTGGAGGCTGAGGATTTGGGAGTTTATTATTGCTGGCAAGGT
ACACATTTTCCGTACACGTTCGGAGGGGGGACCAAGGTGGAAATAAAACGGGCTGATGCTGCACCAACTGTA
TCCATCTTCCCACCATCCAGTGAGCAGTTAACATCTGGAGGTGCCTCAGTCGTGTGCTTCTTGAACAACTTC
TACCCCAAA
VL2HB.4 protein
(SEQ ID NO: 145)
DIVMTQTPLTLSVTIGQPASISCKSSQSLLDSDGKTYLNWLLQRPGQSPKRLIYLVSKLDSGVPDRFTGSGS
GTDETLKISRVEAEDLGVYYCWQGTHFPYTFGGGTKVEIKRADAAPTVSIFPPSSEQLTSGGASVVCFINNE
YPR
VL2HB.4 nucleic acid
(SEQ ID NO: 157)
GATATTGTGATGACCCAGACTCCACTCACTTTGTCGGTTACCATTGGACAGCCAGCCTCCATCTCTTGCAAG
TCAAGTCAGAGCCTCTTAGATAGTGATGGAAAGACATATTTGAATTGGTTGTTACAGAGGCCAGGCCAGTCT
CCAAAGCGCCTAATCTATCTGGTGTCTAAACTGGACTCTGGAGTCCCTGACAGGTTCACTGGCAGTGGATCA
GGGACAGATTTCACACTGAAAATCAGCAGAGTGGAGGCTGAGGATTTGGGAGTTTATTATTGCTGGCAAGGT
ACACATTTTCCGTACACGTTCGGAGGGGGGACCAAGGTGGAAATAAAACGGGCTGATGCTGCACCAACTGTA
TCCATCTTCCCACCATCCAGTGAGCAGTTAACATCTGGAGGTGCCTCAGTCGTGTGCTTCTTGAACAACTTC
TACCCCAGA
VL2HB.7 protein
(SEQ ID NO: 146)
DIVMTQTPLTLSVTIGQPASISCKSSQSLLDSDGKTYLNWLLQRPGQSPKRLIYLVSKLDSGVPDRFTGSGS
GTDFTLKISRVEAEDLGVYYCWQGTHEPYTFGGGTKVEIKRADAAPTVSIFPPSSEQLTSGGASVVCFLNNF
YPR
VL2HB.7 nucleic acid
(SEQ ID NO: 158)
GATATTGTGATGACCCAGACTCCACTCACTTTGTCGGTTACCATTGGACAGCCAGCCTCCATCTCTTGCAAG
TCAAGTCAGAGCCTCTTAGATAGTGATGGAAAGACATATTTGAATTGGTTGTTACAGAGGCCAGGCCAGTCT
CCAAAGCGCCTAATCTATCTGGTGTCTAAACTGGACTCTGGAGTCCCTGACAGGTTCACTGGCAGTGGATCA
GGGACAGATTTCACACTGAAAATCAGCAGAGTGGAGGCTGAGGATTTGGGAGTTTATTATTGCTGGCAAGGT
ACACATTTTCCGTACACGTTCGGAGGGGGGACCAAGGTGGAAATAAAACGGGCTGATGCTGCACCAACTGTA
TCCATCTTCCCACCATCCAGTGAGCAGTTAACATCTGGAGGTGCCTCAGTCGTGTGCTTCTTGAACAACTTC
TACCCCAGA
Consensus
(positions in parenthesis indicate potential
substitutions at that position)
(SEQ ID NO: 147)
D(I/V)VMTQ(T/S)PL(T/S)(L/S)(S/P)VT(I/L/P)GQPASISC(K/R)SSQSLLDSDGKTYLNW(L
/F/E/Y)(L/Q)Q(R/K)PGQ(S/P)P(K/R/Q)(R/L)LIYLVSKLDSGVPDRE(T/S)GSG(S/A)GTDF
TLKISRVE(A/P)ED(L/V)GVYYCWQGTHEPYTFG(G/Q)GTK(V/L)E(I/V)KRADAAPTVSIFPPSSE
QLTSGGASVVCFLNNFYPR

Consensus sequence in which the most common amino acids are indicated at each position (SEQ ID NO: 148) (variable domain is highlighted in bold, with sequencing primers underlined, and CDRs double underlined)

DIVMTQTPLTLSVTIGQPASISCKSSQSLLDSDGKTYLNWLLQRPG
QSPKRLIYLVSKLDSGVPDRFTGSGSGTDFTLKISRVEAEDLGVYY
CWQGTHEPYTFGGGTKVEIKRADAAPTVSIFPPSSEQLTSGGASV
VCFLNNFYPR

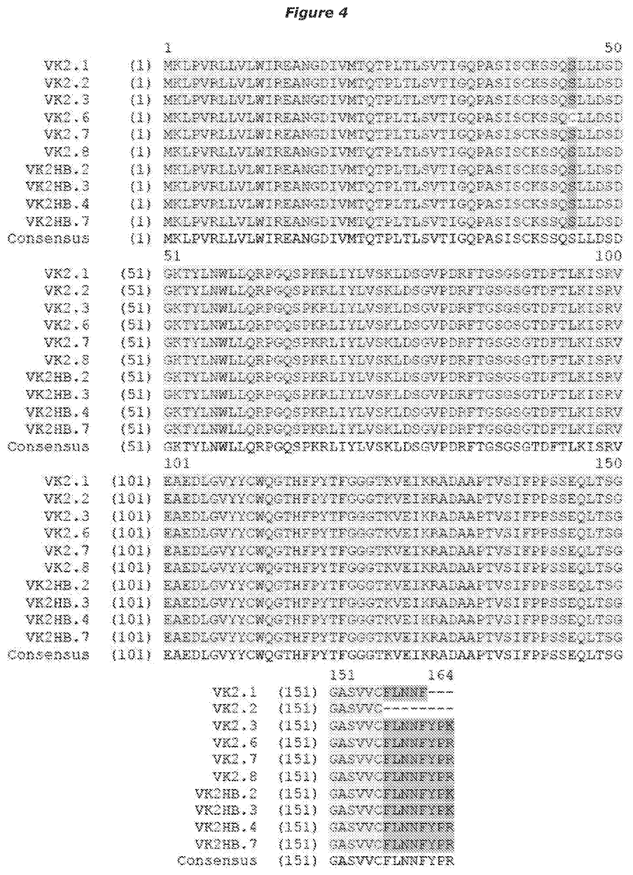

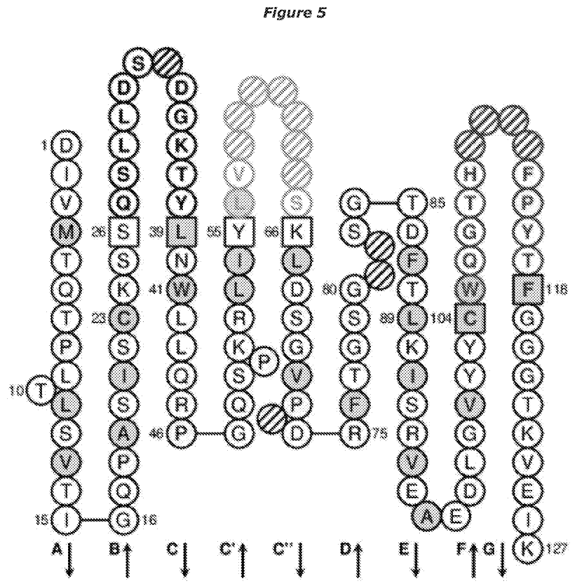

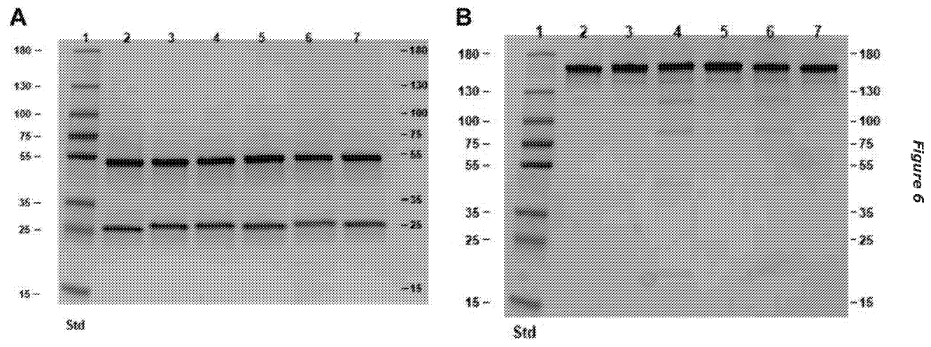

The CDRs indicated by double underlining were determined by the IMGT numbering system (Lefranc, M.-P. et al., Nucleic Acids Research, 27, 209-272 (1999)). The consensus amino acid sequence from VH sequencing was obtained from at least 5 amino acid sequences as shown by the alignment in FIG. 4. A graphical representation of the variable domain with IMGT labelling is shown in FIG. 5.

### Example 2: Transient Expression, Purification and Affinity Screening of Ab 1126 Humanised Anti-VEGF-A165b Clone mα165b (Also Referred to Herein as 56/8/31/1/16) Variants (25+1)

### Summary

Ab 1126 humanized variants are 150 kilodalton (kDa) antibodies composed of two heavy chains and two light chains, complexed together via disulphide bonds. A mammalian expression vector encoding each variant was transfected into CHO cells and batch cultures of each variant grown for up to seven days. The expressed antibodies were purified from cell culture supernatant via affinity chromatography. The concentration and purity were determined for the purified antibody products, Results from these quality control experiments are detailed below.

### Expression and Purification of Ab 1126

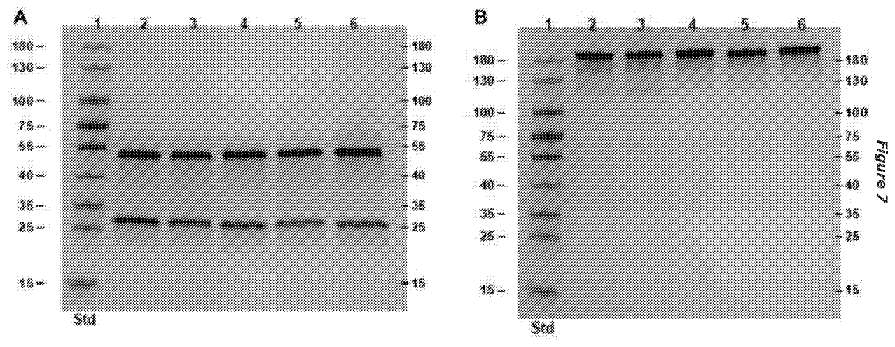

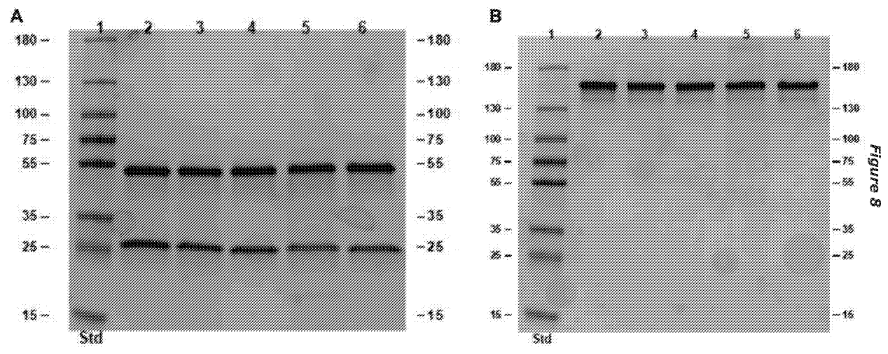

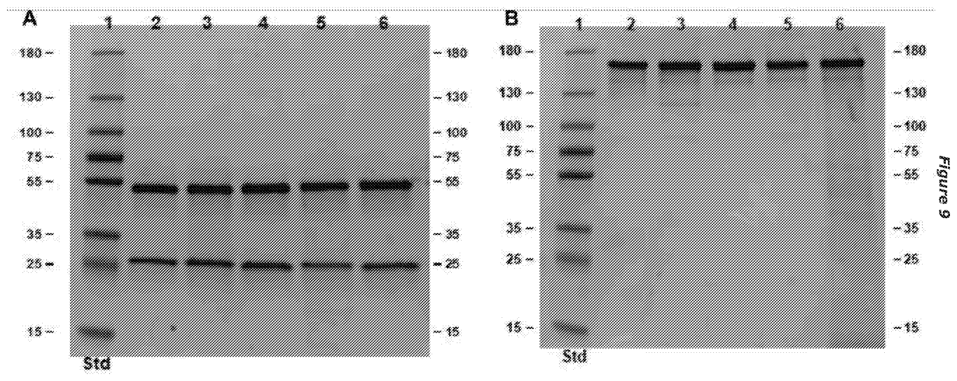

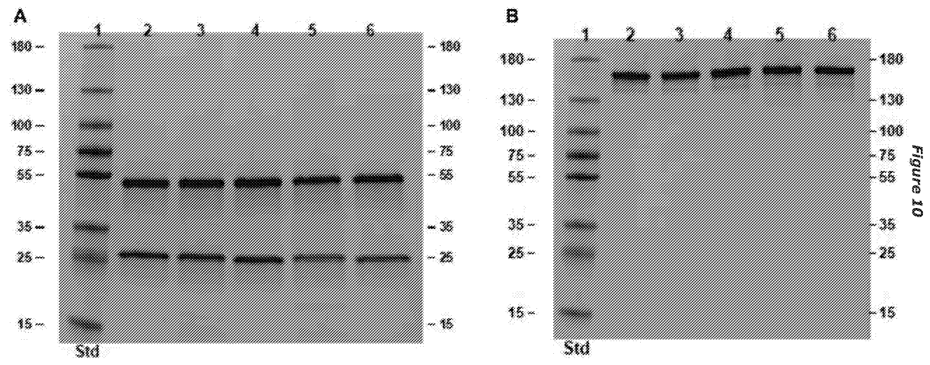

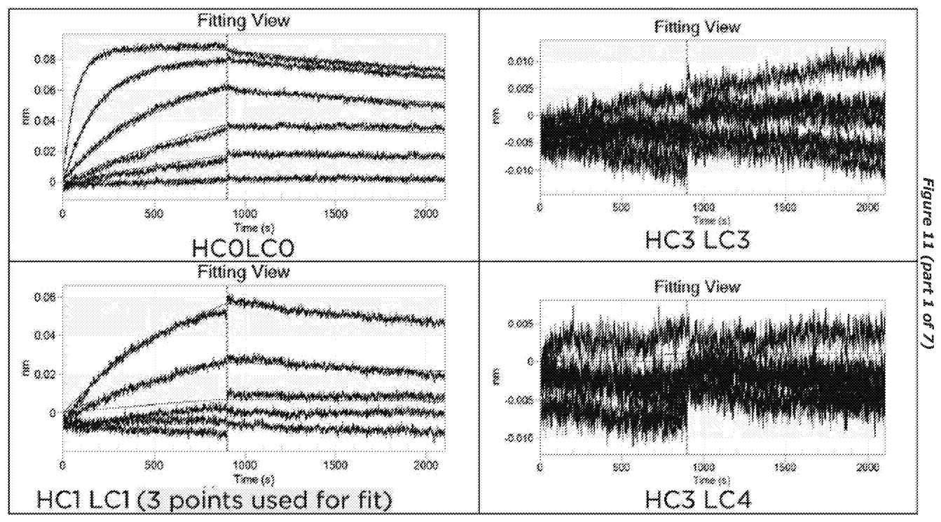

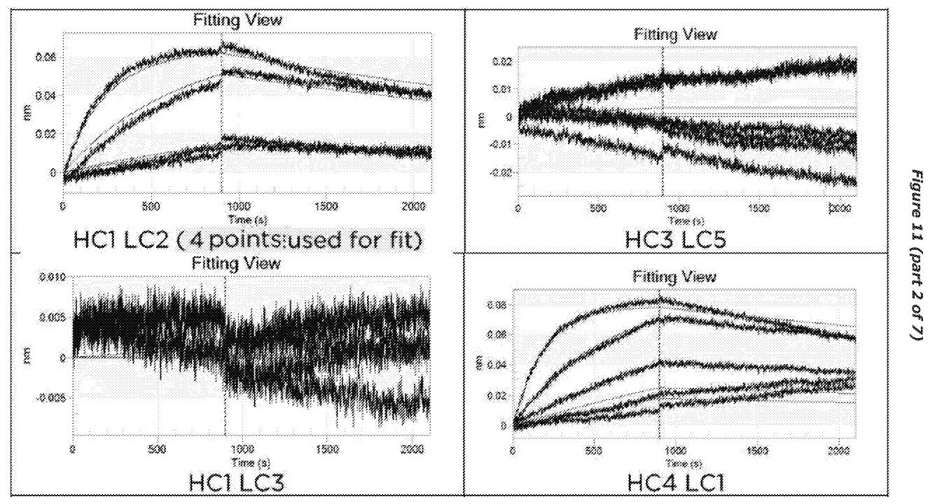

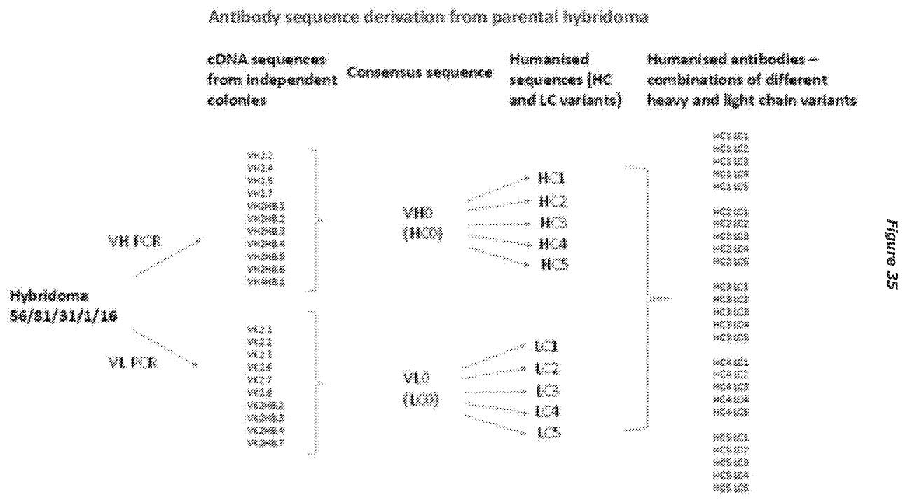

DNA coding for the amino acid sequence of the Ab 1126 variants (see sequences below) were synthesised and cloned into the mammalian transient expression plasmid pETE V2 (property of Fusion antibodies). Ab 1126 variants were expressed using a CHO based transient expression system and the resulting antibody containing cell culture supernatants were clarified by centrifugation and filtration. Ab 1126 variants were purified (using state-of-the-art AKTA chromatography equipment) from cell culture supernatants via affinity chromatography. Purified antibody was dialyzed/buffer exchanged into phosphate buffered saline solution. The purity of the antibody was determined to be >95%, as judged by reducing and denaturing Sodium Dodecyl Sulfate Polyacrylamide gels (FIG. 6-10). Antibody concentration was determined by measuring absorbance at 280 nm and calculated using the standard extinction coefficient 205,500 M⁻¹ cm⁻¹ (or 1.0 mg/ml=A280 of 1.37 [assuming a MW=150,000 Da]) for an antibody. Details of the supplied purified antibody product are summarized in Table 1; and antibody sequence derivation from parental hybridomas is summarised in FIG. 35.

Ab 1126 variant sequences (the signal peptides used for expression of the antibody heavy and light chains are underlined, which may be excluded from the heavy and light chain sequences):

>Ab 1126 HCQ
(SEQ ID NO: 71, with signal peptide)
MGWTLVFLFLLSVTAGVHSEVKLLESGGGLVQPGGSLKLSCAASGEDESRYWMSWVRQAPGKGLEWIGE
IHPYSSTINYTPSVKDKFIISRDNAKNTLYLQMSEVRSEDTALYYCARAFAYWGQGTLVTVSAASTKGP
SVEPLAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVFLFPPKPKDTLMISRTPEVTC
VVVDVSHEDPEVKENWYVDGVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK
>Ab 1126 HCQ
(SEQ ID NO: 125, without signal peptide)
EVKLLESGGGLVQPGGSLKLSCAASGEDESRYWMSWVRQAPGKGLEWIGEIHPYSSTINYTPSVKDKFI
ISRDNAKNTLYLQMSEVRSEDTALYYCARAFAYWGQGTLVTVSAASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVTCVVVDVSHEDPEVKENWYVD
GVEVHNAKTKPREEQYNSTYRVVSVLTVLHQDWINGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGK
>Ab 1126 HC1
(SEQ ID NO: 72, with signal peptide)
MGWTLVFLFLLSVTAGVHSEVQLLESGGGLVKPGGSLRLSCAASGEDESRYWMSWIRQAPGKGLEWVSE
IHPYSSTINYTPSVKDRFTISRDNAKNSLYLQMNSLRAEDTAVYYCARAFAYWGQGTLVTVSSASTKGP
SVEPLAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVFLFPPKPKDTLMISRTPEVTC
VVVDVSHEDPEVKENWYVDGVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK
>Ab 1126 HC1
(SEQ ID NO: 126 without signal peptide)
EVQLLESGGGLVKPGGSLRLSCAASGEDFSRYWMSWIRQAPGKGLEWVSEIHPYSSTINYTPSVKDRET
ISRDNAKNSLYLQMNSLRAEDTAVYYCARAFAYWGQGTLVTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVTCVVVDVSHEDPEVKENWYVD
GVEVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGK
>Ab 1126 HC2
(SEQ ID NO: 73, with signal peptide)
MGWTLVFLFLLSVTAGVHSEVQLVESGGGLVQPGGSLRLSCAASGFDFSRYWMSWVRQAPGKGLEWVAE
IHPYSSTINYTPSVKDRFIISRDNAKNSVYLQLNSLRAEDTAVYYCARAFAYWGQGTLVTVSSASTKGP
SVFPLAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVFLFPPKPKDTLMISRTPEVTC
VVVDVSHEDPEVKENWYVDGVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK
>Ab 1126 HC2
(SEQ ID NO: 127 without signal peptide)
EVQLVESGGGLVQPGGSLRLSCAASGEDESRYWMSWVRQAPGKGLEWVAEIHPYSSTINYTPSVKDRFI
ISRDNAKNSVYLQLNSLRAEDTAVYYCARAFAYWGQGTLVTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVTCVVVDVSHEDPEVKENWYVD
GVEVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGK
>Ab 1126 HC3
(SEQ ID NO: 74, with signal peptide)
MGWTLVFLFLLSVTAGVHSEVQLVESGGGLVQPGGSLRLSCAASGEDESRYWMSWVRQAPGKGLEWISE
IHPYSSTINYTPSVKDRFTISRDNAKNSLYLQMNSLRDEDTALYYCARAFAYWGQGTLVTVSSASTKGP
SVFPLAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVFLFPPKPKDTLMISRTPEVTC
VVVDVSHEDPEVKENWYVDGVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK
>Ab 1126 HC3
(SEQ ID NO: 128 without signal peptide)
EVQLVESGGGLVQPGGSLRLSCAASGEDESRYWMSWVRQAPGKGLEWISEIHPYSSTINYTPSVKDRET
ISRDNAKNSLYLQMNSLRDEDTALYYCARAFAYWGQGTLVTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKV
DKKVEPKSCDKTHTCPPCPAPELLGGPSVELEPPKPKDTLMISRTPEVTCVVVDVSHEDPEVKENWYVD
GVEVHNAKTKPREEQYNSTYRVVSVLTVLHQDWINGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGK
>Ab 1126 HC4
(SEQ ID NO: 75, with signal peptide)
MGWTLVFLFLLSVTAGVHSEVQLVESGGGLVQPGGSLRLSCAASGEDESRYWMSWVRQAPGKGLEWVAE
IHPYSSTINYTPSVKDRETISRDNAKNSLYLQMNSLRSEDTAVYYCARAFAYWGQGTLVTVSSASTKGP
SVFPLAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVFLFPPKPKDTLMISRTPEVTC
VVVDVSHEDPEVKENWYVDGVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVEWESNGQPENNYKTTPPVLD
SDGS FFLYSKLTVDKSRWQQGNVFSCSVMHEALHNHYTQKSLSLSPGK
>Ab 1126 HC4
(SEQ ID NO: 129 without signal peptide)
EVQLVESGGGLVQPGGSLRLSCAASGEDFSRYWMSWVRQAPGKGLEWVAEIHPYSSTINYTPSVKDRET
ISRDNAKNSLYLQMNSLRSEDTAVYYCARAFAYWGQGTLVTVSSASTKGPSVFPLAPSSKSTSGGTAAL
GCLVKDYFPEPVTVSWNSGALTSGVHTFPAVLQSSGLYSLSSVVTVPSSSLGTQTYI CNVNHKPSNTKV
DKKVEPKSCDKTHTCPPCPAPELLGGPSVFLFPPKPKDTLMISRTPEVTCVVVDVSHEDPEVKENWYVD
GVEVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGK
>Ab 1126 HC5
(SEQ ID NO: 76, with signal peptide)
MGWTLVFLFLLSVTAGVHSEVQLVESGGGLVQPGGSLRLSCAASGFDFSRYWMSWVRQAPGKGLEWVSE
IHPYSSTINYTPSVKDRFTISRDNAKNSLYLQMNSLRAEDTAVYYCARAFAYWGQGTLVTVSAASTKGP
SVFPLAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWINGKEYKCKVSNKALPA
PIEKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK
>Ab 1126 HC5
(SEQ ID NO: 130 without signal peptide)
EVQLVESGGGLVQPGGSLRLSCAASGFDFSRYWMSWVRQAPGKGLEWVSEIHPYSSTINYTPSVKDRET
ISRDNAKNSLYLQMNSLRAEDTAVYYCARAFAYWGQGTLVTVSAASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVTCVVVDVSHEDPEVKENWYVD
GVEVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGK
>Ab 1126 LCQ
(SEQ ID NO: 77, with signal peptide)
MVSSAQFLGLLLLCFQGTRCDIVMTQTPLTLSVTIGQPASISCKSSQSLLDSDGKTYLNWLLQRPGQSP
KRLIYLVSKLDSGVPDRFTGSGSGTDFTLKISRVEAEDLGVYYCWQGTHEPYTFGGGTKVEIKRTVAAP
SVFIFPPSDEQLKSGTASVVCLLNNFYPREAKVQWKVDNALQSGNSQESVTEQDSKDSTYSLSSTLTLS
KADYEKHKVYACEVTHQGLSSPVTKSENRGEC
>Ab 1126 LCQ
(SEQ ID NO: 131 without signal peptide)
DIVMTQTPLTLSVTIGQPASISCKSSQSLLDSDGKTYLNWLLQRPGQSPKRLIYLVSKLDSGVPDRETG
SGSGTDFTLKISRVEAEDLGVYYCWQGTHFPYTFGGGTKVEIKRTVAAPSVFIFPPSDEQLKSGTASVV
CLLNNFYPREAKVQWKVDNALQSGNSQESVTEQDSKDSTYSLSSTLTLSKADYEKHKVYACEVTHQGLS
SPVTKSENRGEC
>Ab 1126 LC1
(SEQ ID NO: 78, with signal peptide)
MVSSAQFLGLLLLCFQGTRCDVVMTQSPLSLPVTLGQPASISCKSSQSLLDSDGKTYLNWFQQRPGQSP
RRLIYLVSKLDSGVPDRFSGSGSGTDFTLKISRVEAEDVGVYYCWQGTHFPYTFGGGTKVEIKRTVAAP
SVFIFPPSDEQLKSGTASVVCLINNFYPREAKVQWKVDNALQSGNSQESVTEQDSKDSTYSLSSTLTLS
KADYEKHKVYACEVTHQGLSSPVTKSENRGEC
>Ab 1126 LC1
(SEQ ID NO: 132 without signal peptide)
DVVMTQSPLSLPVTLGQPASISCKSSQSLLDSDGKTYLNWFQQRPGQSPRRLIYLVSKLDSGVPDRFSG
SGSGTDFTLKISRVEAEDVGVYYCWQGTHFPYTFGGGTKVEIKRTVAAPSVFIFPPSDEQLKSGTASVV
CLINNFYPREAKVQWKVDNALQSGNSQESVTEQDSKDSTYSLSSTLTLSKADYEKHKVYACEVTHQGLS
SPVTKSENRGEC
>Ab 1126 LC2
(SEQ ID NO: 79, with signal peptide)
MVSSAQFLGLLLLCFQGTRCDVVMTQSPLSLPVTLGQPASISCRSSQSLLDSDGKTYLNWFLQRPGQSP
RRLIYLVSKLDSGVPDRESGSGSGTDFTLKISRVEAEDVGVYYCWQGTHEPYTFGQGTKVEIKRTVAAP
SVFIFPPSDEQLKSGTASVVCLINNFYPREAKVQWKVDNALQSGNSQESVTEQDSKDSTYSLSSTLTLS
KADYEKHKVYACEVTHQGLSSPVTKSENRGEC
>Ab 1126 LC2
(SEQ ID NO: 133 without signal peptide)
DVVMTQSPLSLPVTLGQPASISCRSSQSLLDSDGKTYLNWFLQRPGQSPRRLIYLVSKLDSGVPDRESG
SGSGTDFTLKISRVEAEDVGVYYCWQGTHFPYTFGQGTKVEIKRTVAAPSVFIFPPSDEQLKSGTASVV
CLLNNFYPREAKVQWKVDNALQSGNSQESVTEQDSKDSTYSLSSTLTLSKADYEKHKVYACEVTHQGLS
SPVTKSENRGEC
>Ab 1126 LC3
(SEQ ID NO: 80, with signal peptide)
MVSSAQFLGLLLLCFQGTRCDIVMTQTPLSLSVTPGQPASISCKSSQSLLDSDGKTYLNWYLQKPGQSP
QLLIYLVSKLDSGVPDRFSGSGSGTDFTLKISRVEPEDVGVYYCWQGTHEPYTFGGGTKVEVKRTVAAP
SVFIFPPSDEQLKSGTASVVCLLNNFYPREAKVQWKVDNALQSGNSQESVTEQDSKDSTYSLSSTLTLS
KADYEKHKVYACEVTHQGLSSPVTKSENRGEC
>Ab 1126 LC3
(SEQ ID NO: 134 without signal peptide)
DIVMTQTPLSLSVTPGQPASISCKSSQSLLDSDGKTYLNWYLQKPGQSPQLLIYLVSKLDSGVPDRESG
SGSGTDFTLKISRVEPEDVGVYYCWQGTHFPYTFGGGTKVEVKRTVAAPSVFIFPPSDEQLKSGTASVV
CLLNNFYPREAKVQWKVDNALQSGNSQESVTEQDSKDSTYSLSSTLTLSKADYEKHKVYACEVTHQGLS
SPVTKSENRGEC
>Ab 1126 LC4
(SEQ ID NO: 81, with signal peptide)
MVSSAQFLGLLLLCFQGTRCDIVMTQTPLSSPVTLGQPASISCRSSQSLLDSDGKTYLNWLQQRPGQPP
RLLIYLVSKLDSGVPDRESGSGAGTDETLKISRVEAEDVGVYYCWQGTHFPYTFGQGTKLEIKRTVAAP
SVFIFPPSDEQLKSGTASVVCLLNNFYPREAKVQWKVDNALQSGNSQESVTEQDSKDSTYSLSSTLTLS
KADYEKHKVYACEVTHQGLSSPVTKSENRGEC
>Ab 1126 LC4
(SEQ ID NO: 135 without signal peptide)
DIVMTQTPLSSPVTLGQPASISCRSSQSLLDSDGKTYLNWLQQRPGQPPRLLIYLVSKLDSGVPDRESG
SGAGTDFTLKISRVEAEDVGVYYCWQGTHEPYTFGQGTKLEIKRTVAAPSVFIFPPSDEQLKSGTASVV
CLINNFYPREAKVQWKVDNALQSGNSQESVTEQDSKDSTYSLSSTLTLSKADYEKHKVYACEVTHQGLS
SPVTKSENRGEC
>Ab 1126 LC5
(SEQ ID NO: 82, with signal peptide)
MVSSAQFLGLLLLCFQGTRCDIVMTQTPLSLSVTPGQPASISCKSSQSLLDSDGKTYLNWYLQKPGQSP
QLLIYLVSKLDSGVPDRFSGSGSGTDFTLKISRVEAEDVGVYYCWQGTHFPYTFGGGTKVEIKRTVAAP
SVFIFPPSDEQLKSGTASVVCLINNFYPREAKVQWKVDNALQSGNSQESVTEQDSKDSTYSLSSTLTLS
KADYEKHKVYACEVTHQGLSSPVTKSENRGEC
>Ab 1126 LC5
(SEQ ID NO: 136 without signal peptide)
DIVMTQTPLSLSVTPGQPASISCKSSQSLLDSDGKTYLNWYLQKPGQSPQLLIYLVSKLDSGVPDRESG
SGSGTDFTLKISRVEAEDVGVYYCWQGTHFPYTFGGGTKVEIKRTVAAPSVFIFPPSDEQLKSGTASVV
CLLNNFYPREAKVQWKVDNALQSGNSQESVTEQDSKDSTYSLSSTLTLSKADYEKHKVYACEVTHQGLS
SPVTKSENRGEC

TABLE 1
Purification summary: Ab 1126 variants. Abbreviations
are as follows: ND = not determined. *Purity
determined by (relative band intensity) analysis of
Coomassie blue stained reducing and denaturing SDS-PAGE.

| | | | | Potential | |
|---|---|---|---|---|---|
| | Concentration | Volume | Total | yield | |
| Sample | (mg/mL) | (mL) | (mg) | (mg/L) | *Purity |
| HC0 LC0 | 0.64 | 1.66 | 1.06 | 42.40 | ≥95% |
| HC1 LC1 | 0.77 | 1.70 | 1.31 | 52.40 | ≥95% |
| HC1 LC2 | 0.58 | 1.71 | 0.99 | 39.60 | ≥95% |
| HC1 LC3 | 0.72 | 1.74 | 1.25 | 50.00 | ≥95% |
| HC1 LC4 | 0.71 | 1.74 | 1.24 | 49.60 | ≥95% |
| HC1 LC5 | 1.00 | 1.79 | 1.79 | 71.60 | ≥95% |
| HC2 LC1 | 1.04 | 1.83 | 1.90 | 76.00 | ≥95% |
| HC2 LC2 | 0.89 | 1.93 | 1.72 | 68.80 | ≥95% |
| HC2 LC3 | 0.63 | 2.96 | 1.89 | 75.60 | ≥95% |
| HC2 LC4 | 0.99 | 1.89 | 1.87 | 74.80 | ≥95% |
| HC2 LC5 | 0.72 | 1.86 | 1.36 | 53.60 | ≥95% |
| HC3 LC1 | 1.05 | 1.97 | 2.07 | 82.80 | ≥95% |
| HC3 LC2 | 0.85 | 1.99 | 1.69 | 67.60 | ≥95% |
| HC3 LC3 | 0.57 | 1.35 | 0.77 | 51.30 | ≥95% |
| HC3 LC4 | 0.50 | 1.50 | 0.75 | 30.00 | ≥95% |
| HC3 LC5 | 0.86 | 1.47 | 1.26 | 50.40 | ≥95% |
| HC4 LC1 | 0.89 | 1.57 | 1.40 | 56.00 | ≥95% |
| HC4 LC2 | 1.30 | 1.68 | 2.18 | 87.20 | ≥95% |
| HC4 LC3 | 1.00 | 1.79 | 1.79 | 71.60 | ≥95% |
| HC4 LC4 | 0.73 | 1.88 | 1.39 | 54.80 | ≥95% |
| HC4 LC5 | 0.87 | 1.61 | 1.40 | 56.00 | ≥95% |
| HC5 LC1 | 1.13 | 1.90 | 2.15 | 86.00 | ≥95% |
| HC5 LC2 | 0.93 | 1.91 | 1.78 | 71.20 | ≥95% |
| HC5 LC3 | 0.84 | 1.61 | 1.35 | 54.00 | ≥95% |
| HC5 LC4 | 0.81 | 1.81 | 1.47 | 58.80 | ≥95% |
| HC5 LC5 | 0.79 | 1.82 | 1.44 | 57.60 | ≥95% |

### Kinetic Analysis: Summary

IgG antibodies were immobilized on biosensors using suitable capture surfaces and binding of soluble antigen to immobilized antibodies was monitored by BLI (Octet). The resulting sensorgrams were analyzed using the manufacturer's supplied software (Fortebio).

### Kinetic Analysis: Materials and Methods

Antigen: Human VEGF165b, Sino Biological, Catalog No. 29656-HNAB, Lot #LC13JU1402 **NOTE ** VEGF165b often exists as a disulfide-linked homodimer

Details of antibodies tested:
Antibody identifier=HCX LCX
Species=Humanized
Capture method=Anti-human IgG Fc (AHC)
   Kinetic analysis: Optimisation

A series of experiments were initially performed to optimize assay parameters (data not shown), resulting in the following parameters:
Summary of optimized kinetic screening parameters for the characterisation of IgG: antigen interactions

| Antibody loading concentration | 0.15 | μg/mL |
|---|---|---|
| Antigen stability at 25 °C, shaking at 1000 rpm | <4 | hours |
| Time for association/ dissociation | 900/1200 | seconds |
| Kinetic screening protocol required | Modified | |
| Antigen screening range (seven concentration, | 30-0.0412 | nM |
| dilute 3-fold) | | |

| | |
|---|---|
| Interaction/fitting model | 1:1 |

From standard assay optimisation experiments, using the chimeric control antibody, very slow dissociation rates (kd) were observed (<1.0×10⁻⁴) for the antigen when using standard IgG capture levels (0.5-1.0 nm).

### Kinetic Analysis: Experimental Parameters

Kinetic assays were performed by first capturing IgG using anti-human Fc biosensors. The mAb capture biosensors were then submerged in wells containing different concentrations of antigen (association stage) followed by a dissociation step in running buffer. To allow for reference correction, IgG-captured sensors were dipped into wells containing only buffer. This referencing provided a means of compensating for the natural dissociation of the capture IgG. Steps were performed at 25 °C at a constant flow-rate of 1000 rpm. New sensors were used for each sample. Dissociation rate constants (K_{D}) were calculated using the ForteBio Data Analysis software. All consumables used were those recommended by ForteBio.

### Kinetic Analysis: Results for Ab 1126 Variants

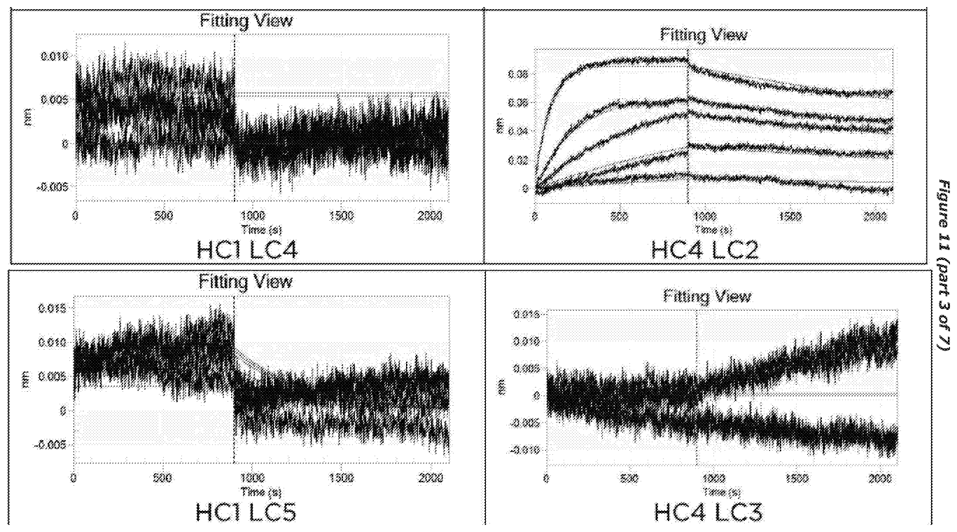

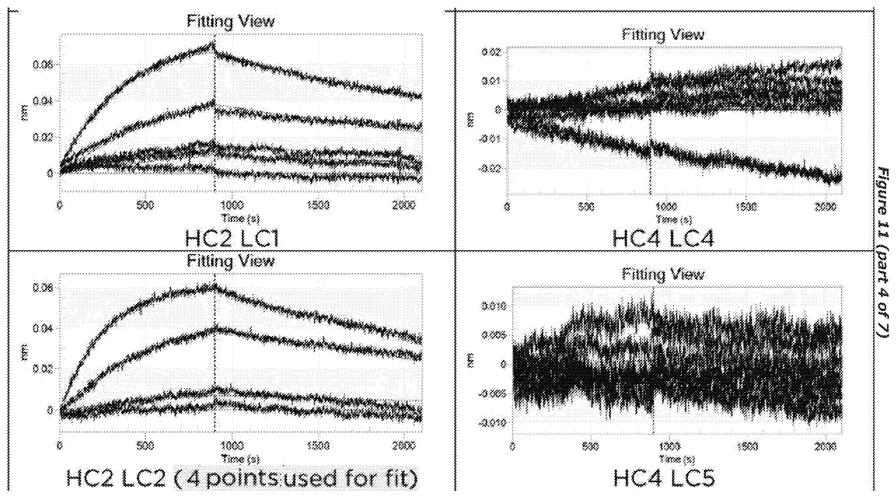

All samples were diluted in freshly prepared running buffer. Antibody variants were immobilized onto the surface of a series of biosensors using the capture methods described (see materials and methods). Antigen was passed over the surface to generate a binding response. Binding data for the IgG: antigen interactions were collected at 25 °C on the biosensors. A dilution series of the antigen was used in the association step, in order to fit results globally and get the best values for ka, kd, and K_{D}. The response data for the binding of antigen to the surface immobilized IgG were fitted to a 1:1 binding model, yielding the data traces (red-see FIG. 11). Kinetic parameters are summarized in Table 2 below.

TABLE 2
Kinetic parameters (fit to 1:1 interaction model)
for Ab 1126 variants and antigen interaction.

| | Capture | | | | | | |
|---|---|---|---|---|---|---|---|
| | level | kₐ | k_{d} | K_{D} | | | Mean |
| Antibody | (nm) | (m⁻¹s⁻¹) | (s⁻¹) | (nM) | R² | X² | Rₘₐₓ |
| HC0 LC0 | 0.595 | 5.06E+05 | 1.35E−04 | 0.3 | 0.99 | 0.05 | 0.08 |
| HC1 LC1 | 0.620 | 3.34E+04 | 1.67E−04 | 5.0 | 0.98 | 0.05 | 0.10 |
| HC1 LC2 | 0.632 | 1.26E+05 | 2.63E−04 | 2.1 | 0.98 | 0.09 | 0.08 |
| HC1 LC3 | 0.671 | NF | NF | NF | NF | NF | NF |
| HC1 LC4 | 0.601 | NF | NF | NF | NF | NF | NF |
| HC1 LC5 | 0.659 | NF | NF | NF | NF | NF | NF |
| HC2 LC1 | 0.669 | 9.38E+04 | 4.16E−04 | 4.4 | 0.99 | 0.05 | 0.09 |
| HC2 LC2 | 0.626 | 1.17E+05 | 4.22E−04 | 3.6 | 0.99 | 0.03 | 0.04 |
| HC2 LC3 | 0.660 | NF | NF | NF | NF | NF | NF |
| HC2 LC4 | 0.634 | NF | NF | NF | NF | NF | NF |
| HC2 LC5 | 0.681 | NF | NF | NF | NF | NF | NF |
| HC3 LC1 | 0.646 | 1.62E+05 | 2.47E−04 | 1.5 | 0.98 | 0.10 | 0.11 |
| HC3 LC2 | 0.622 | 1.71E+05 | 2.00E−04 | 1.17 | 0.99 | 0.05 | 0.08 |
| HC3 LC3 | 0.673 | NF | NF | NF | NF | NF | NF |
| HC3 LC4 | 0.641 | NF | NF | NF | NF | NF | NF |
| HC3 LC5 | 0.642 | NF | NF | NF | NF | NF | NF |
| HC4 LC1 | 0.573 | 1.70E+05 | 1.49E−04 | 0.9 | 0.98 | 0.15 | 0.16 |
| HC4 LC2 | 0.613 | 4.22E+05 | 2.65E−04 | 0.6 | 0.99 | 0.08 | 0.08 |
| HC4 LC3 | 0.688 | NF | NF | NF | NF | NF | NF |
| HC4 LC4 | 0.669 | NF | NF | NF | NF | NF | NF |
| HC4 LC5 | 0.626 | NF | NF | NF | NF | NF | NF |
| HC5 LC1 | 0.657 | 1.19E+05 | 3.81E−04 | 3.20 | 0.99 | 0.04 | 0.20 |
| HC5 LC2 | 0.618 | 2.11E+05 | 1.17E−04 | 0.6 | 1.00 | 0.05 | 0.20 |
| HC5 LC3 | 0.666 | NF | NF | NF | NF | NF | NF |
| HC5 LC4 | 0.650 | NF | NF | NF | NF | NF | NF |
| HC5 LC5 | 0.651 | NF | NF | NF | NF | NF | NF |

R² values indicate how well the fit and experimental data correlate and above 0.95 are considered a good fit; X² is the sum of the squared deviation should be generally below 3 (Ideally <0.5). X² is the measure of error between the experimental data and the fitted line. The smaller the X² indicates a better fit. Mean Rₘₐₓ should be similar (±20%) to that of the chimeric control. K_{D} should be within 2-fold of the chimeric control antibody, HC0LC0. Abbreviations are as follows; NF, does not fit to 1:1 binding model.

### Conclusion

All Ab 1126 variants have been successfully expressed and purified.

From the SDS-PAGE analysis, all antibodies exhibit adequate levels of purity. Under denaturing and reducing conditions, both heavy and the light chains of the antibody are visible and are observed at the expected molecular weight of ~50 and 25 kDa, respectively. Under denaturing and non-reducing conditions, a single major band and several minor bands are observed. The additional bands (impurities) are likely the result of non-glycosylated IgG and IgG degradation products (e.g. a single [partial] light chain, a combination of two heavy and one light chain, two heavy chains, two heavy and one light chain).

Kinetic (Octet) analysis: HCX LC1 and HCX LC2 all exhibit binding to the antigen and in most cases, the experimental data fits well to a 1:1 binding model. Under the experimental conditions used here, HC5 LC2 and HC4 LC2 exhibit binding characteristics similar to that of the chimeric control antibody HC0 LC0, exhibiting dissociation constants within 2-fold of the chimeric HC0 LC0 control antibody.

### Example 3: Transient Expression of Ab 1126 Variants, HC0 LC0, HC4 LC2 and HC5 LC5 (20 mg Scale Up)

### Summary

Ab 1126 variants HC0 LC0, HC4 LC2 and HC5 LC2 are 150 kilodalton (kDa) antibodies composed of two heavy chains and two light chains, complexed together via disulphide bonds. A mammalian expression vector encoding each antibody was transfected into CHO cells. The expressed antibodies were subsequently purified from cell culture supernatant using standard chromatography techniques. The concentration, purity and endotoxin were determined for the purified antibody products.

### Expression and Purification of Ab 1126 Variants

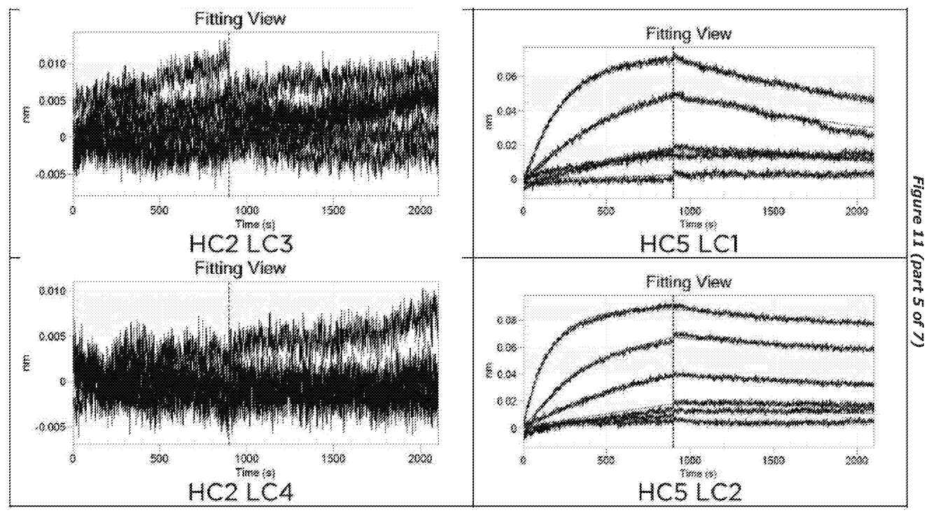

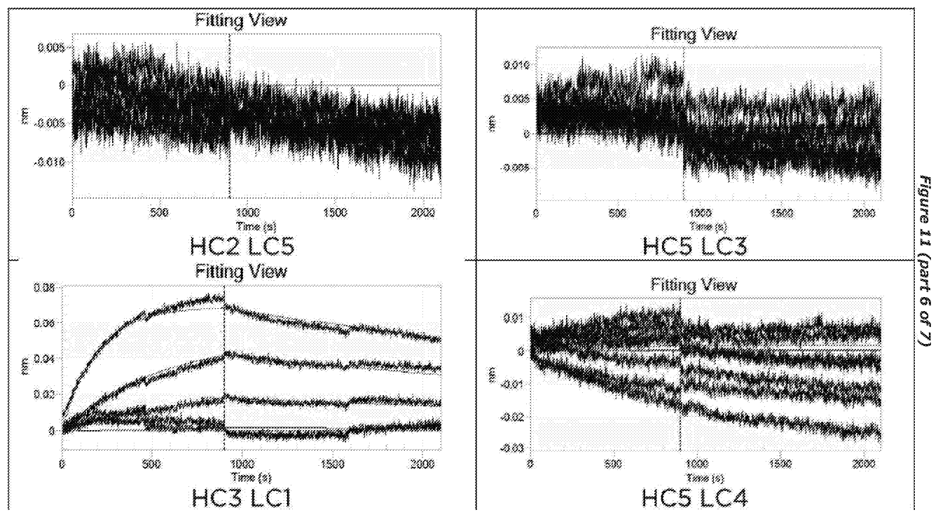

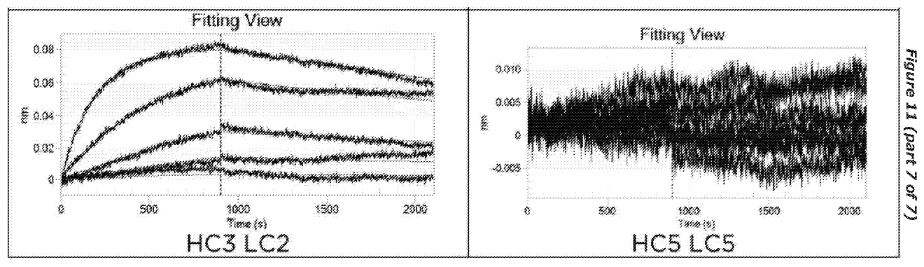

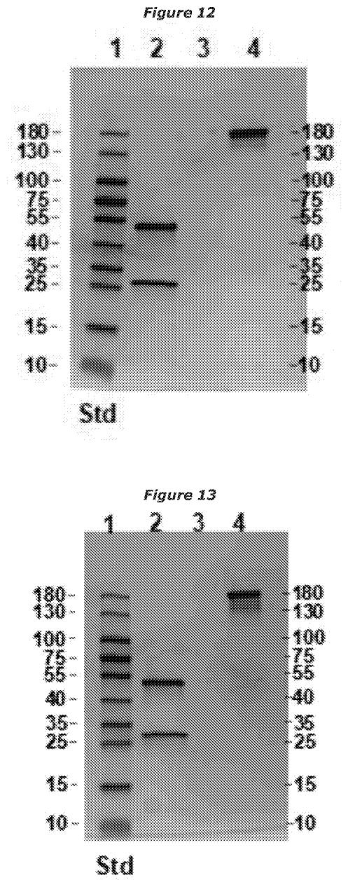

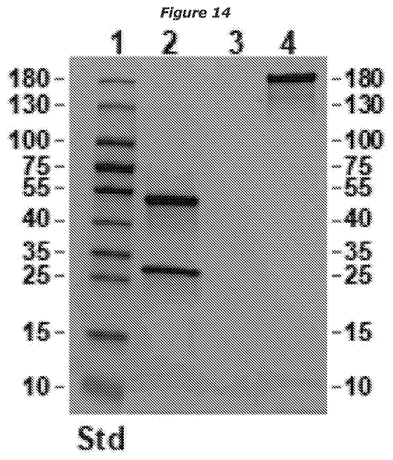

DNA coding for the amino acid sequence of each antibody was synthesised and cloned into the mammalian transient expression plasmid pETE V2 (property of Fusion antibodies). Antibodies were expressed using a CHO based transient expression system and the resulting antibody containing cell culture supernatants were clarified by centrifugation and filtration. Antibodies were purified (using state-of-the-art AKTA chromatography equipment) from cell culture supernatants via affinity chromatography and preparative grade size exclusion chromatography (SEC column was pre-equilibrated in phosphate buffered saline solution). Purified antibodies were buffer exchanged into phosphate buffered saline solution. The purity of the antibodies was determined to be >95%, as judged by reducing and denaturing Sodium Dodecyl Sulfate Polyacrylamide gels (FIG. 12-14).

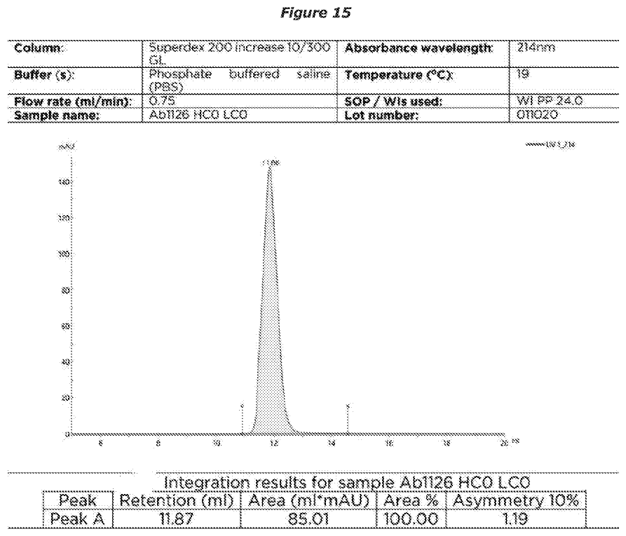

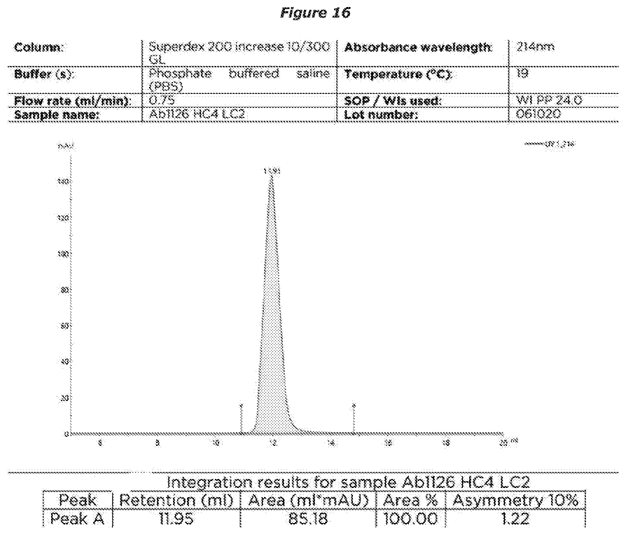

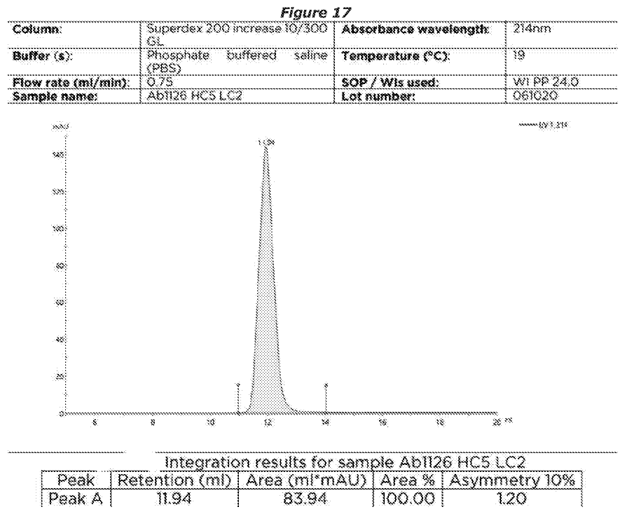

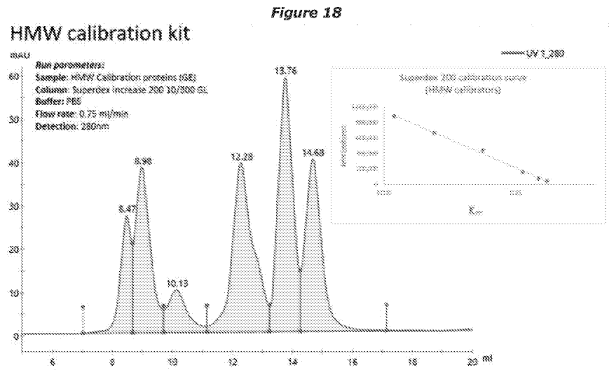

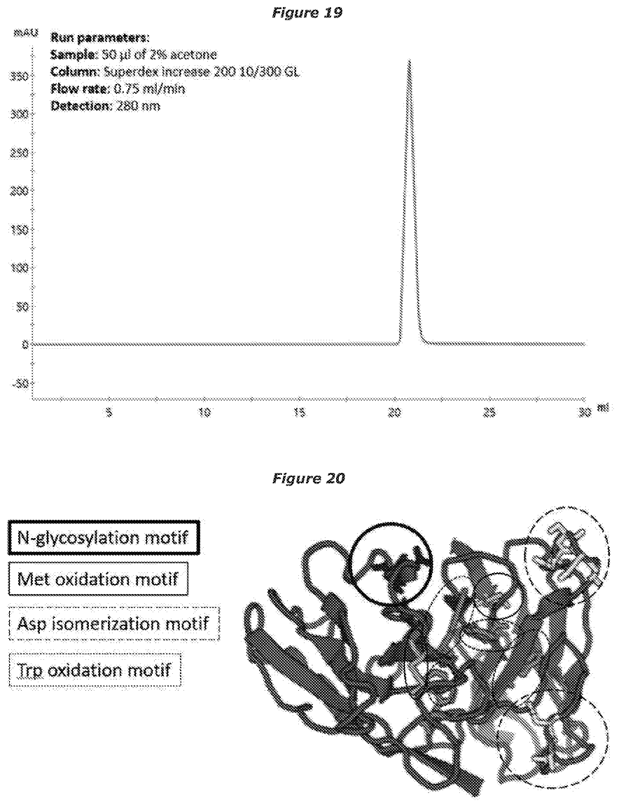

Antibody samples were analysed via size exclusion chromatography (SEC). The chromatograms each show one major peak (>95% total area [FIG. 15-17]). Bacterial endotoxin levels were determined using the Endosafe®-PTS system and Endosafe® PTS cartridges (Charles River Laboratories).

Antibody concentration was determined by measuring absorbance at 280 nm and calculated using the standard extinction coefficient 205,500 M-1 cm-1 (or 1.0 mg/ml=A280 of 1.37 [assuming a MW=150,000 Da]) for an antibody, Details of the supplied purified antibody product are summarized in Table 3.

TABLE 3
Purification summary: Ab 1126 variants.

| | Concen- | | | | Monomer | |
|---|---|---|---|---|---|---|
| | tration | Volume | Total | *Purity | level | Endotoxin |
| Sample | (mg/ml) | (ml) | (mg) | (%) | (%) | (EU/mg) |
| Ab 1126 | 7.33 | 8.90 | 65.24 | >95 | >98 | <1.00 |
| HC0 LC0 | | | | | | |
| Ab 1126 | 3.75 | 14.00 | 52.50 | >95 | >98 | <1.00 |
| HC4 LC2 | | | | | | |
| Ab 1126 | 4.19 | 15.00 | 62.85 | >95 | >98 | <1.00 |
| HC5 LC2 | | | | | | |

*Purity determined by (relative band intensity) analysis of Coomassie blue stained reducing and denaturing SDS polyacrylamide gel.

### Conclusion

Antibodies were successfully expressed and purified.

SEC analyses were performed successfully for all antibodies. The chromatogram shows one major peak (>98% total area).

From the SDS-PAGE analysis, all antibodies exhibit adequate levels of purity, under reducing and denaturing conditions. Under reducing conditions, both heavy and the light chains of the antibody are visible and are observed at the expected molecular weight of ~50 and 25 kDa, respectively. Under non-reducing conditions, a single major band and several minor bands are observed. The additional bands (impurities) are likely the result of non-glycosylated IgG and IgG degradation products (e.g. a single [partial] light chain, a combination of two heavy and one light chain, two heavy chains, two heavy and one light chain).

### Example 4: Evaluation of the Suitability of a Novel Humanised Monoclonal Antibody Against a Specific Isoform of VEGF-A (VEGF-A₁₆₅b) as an Antibody Therapy for Peripheral Artery Disease

### ABBREVIATIONS AND ACRONYMS

| Abbreviation | Definition |
|---|---|
| aSEC | Analytical size exclusion chromatography |
| BLI | Biolayer interferometry |
| CDR | Complementarity determining region |
| CH2 | Constant heavy chain domain 2 |
| CH3 | Constant heavy chain domain 3 |
| CHO | Chines Hamster Ovary |
| CIEF | Capillary Isoelectric focusing |
| CMO | Contract manufacturing organisation |
| CRO | Contract research organisation |
| DLS | Dynamic light scattering |
| DSC | Differential scanning calorimetry |
| DSF | Differential scanning fluorimetry |
| FAb | Fragment antigen binding |
| Fc | Fragment crystallisable |
| FcRn | Neonatal Fc receptor |
| Fv | Fragment variable domains |
| GLP | Good Laboratory Practice |
| HC | Heavy chain |
| HIC | Hydrophobic interaction chromatography |
| HLA | Human Lymphocyte Antigen |
| HPLC | High performance liquid chromatography |
| i.p. | Intraperitoneal |
| i.v. | Intravenous |
| IEDB | Immune Epitope Database |
| IgG1 | Immunoglobulin gamma isotype 1 |
| IMGT | Immunogenetics Information System ® |
| INN | International Non-proprietary Name |
| KD | Dissociation constant |
| KLH | Keyhole limpet haemocyanin |
| LC | Light chain |
| LC-MS | Liquid chromatography - mass spectrometry |
| LPS | Lipopolysaccharide |
| mAb | Monoclonal Antibody |
| MHC | Major Histocompatibility Complex |
| PAD | Peripheral Artery Disease |
| PBS | Phosphate Buffered Saline |
| PK | Pharmacokinetic |
| s.c. | Subcutaneous |
| SDS-PAGE | Sodium dodecyl sulfate polyacrylamide gel electrophoresis |
| SEC | Size exclusion chromatography |
| SPR | Surface plasmon resonance |
| TPP | Target Product Profile |
| VEGF-A | Vascular endothelial growth factor isoform A |
| VH | Heavy chain variable domain |
| VL | Light chain variable domain |
| WHO | World Health Organisation |

### Background

Peripheral Artery Disease (PAD) is a major unmet clinical need afflicting approximately 10 million people in the United States. Vascular insufficiency in PAD results in tissue ischemia. In response, circulating monocytes produce vascular endothelial growth factor (VEGF-A). Patients with PAD and murine models of metabolic ischemia have reduced soluble frizzled related protein 5 (sfrp5) resulting in increased Wnt5a activity resulting in overexpression of the anti-angiogenic isoform, VEGF-A₁₆₅b. It has previously been shown that antibodies to VEGF-A₁₆₅b can reverse impaired revascularisation in mouse models of hindlimb ischemia. Lower-extremity ischemia in PAD is painful and disabling, causing non-healing ulcers and results in 200,000 amputations per year in the United States alone. Limb ischemia induced by arterial obstructive lesions is exacerbated by insufficient angiogenesis and collateral vessel formation.

Recent studies have shown that an anti-angiogenic splice variant of VEGF-A, VEGF-A₁₆₅b is expressed in clinical and experimental settings that are associated with impaired vascularization¹. VEGF-A₁₆₅b is a competitive inhibitor of VEGF-A₁₆₅a mediated angiogenesis. Circulating VEGF-A in patients with PAD is predominantly comprised of the VEGF-A₁₆₅b isoform. In mouse experimental models, mVEGF-A₁₆₅b expression is upregulated under conditions of systemic metabolic dysfunction. The VEGF-A₁₆₅b isoform impairs revascularization in an experimental model of PAD², and acute antibody-mediated neutralization of mVEGF-A₁₆₅b isoform promotes revascularization of ischemic tissue under conditions in which the process of regenerative angiogenesis is impaired. These findings support the concept that VEGF-A₁₆₅b may serve as a new pharmacological target to treat limb ischemia in patients with PAD and that its neutralization may augment the activities of pro-angiogenic growth factors³.

### Introduction

It is an aim to develop a first in class humanised monoclonal antibody directed against the VEGF-A isoform, VEGF-A₁₆₅b for the treatment of PAD. A mouse monoclonal antibody specific for VEGF-A₁₆₅b was generated by immunisation of BALB/c mice with synthetic peptide fragments of the nine amino acid carboxy-terminal sequence of VEGF-A₁₆₅b, the final six of which (CDKPRR) are specific to VEGF-A₁₆₅b, coupled to keyhole limpet haemocyanin (KLH)⁴. One of the resultant hybridomas specifically detected VEGF-A₁₆₅b and not VEGF-A₁₆₅a. Twenty-five humanised variants of one mouse anti-VEGF-A₁₆₅b neutralizing mAb were generated. The variants were transiently expressed in CHO cells and purified from culture supernatants by single column protein A affinity chromatography.

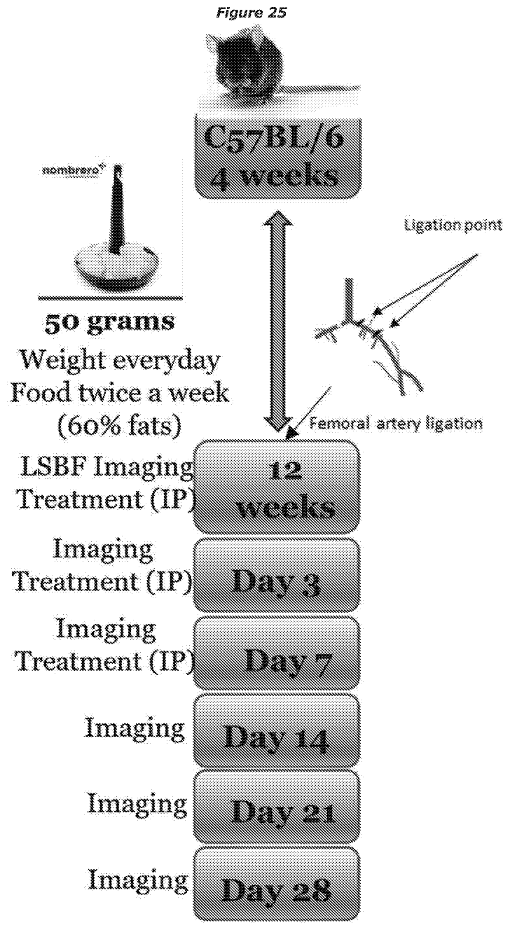

As part of this study, wildtype C57BL6 mice were fed a high fat diet for 12 weeks and underwent left femoral artery ligation. Blood flow to the paw was measured by Moor FLPI-2 Laser Speckle imaging before and after excision of the left femoral artery, and on post-operative days 3, 7, 14, 21 and 28. Animals received humanized anti-VEGF-A₁₆₅b (1 mg/kg, i.p.) or control antibody (human IgG) on day 0, 3 and 7 (see FIG. 25 for a schematic). The ratio of the ischemic vs non-ischemic LDBF was calculated and plotted against time to determine the blood flow recovery. Muscles were stained to measure capillary and arteriole density. Animals where a less than 60% inhibition of blood flow was achieved were excluded. ZDF diabetic rats were similarly treated, and blood flow measured.

### Results

Affinity ranking of the variants was performed by biolayer interferometry (BLI) on commercially available VEGF-A₁₆₅b protein. Two of the variants HC4 LC2 and HC5 LC2 had affinities (KD) which were within two-fold of the HC0 LC0 chimeric control antibody (KD=600, 600 and 300 μM respectively), with new antibodies demonstrating >100× higher affinity than the original mouse antibody to VEGF₁₆₅b, and a slower off rate and equivalent on rate, see FIG. 11.

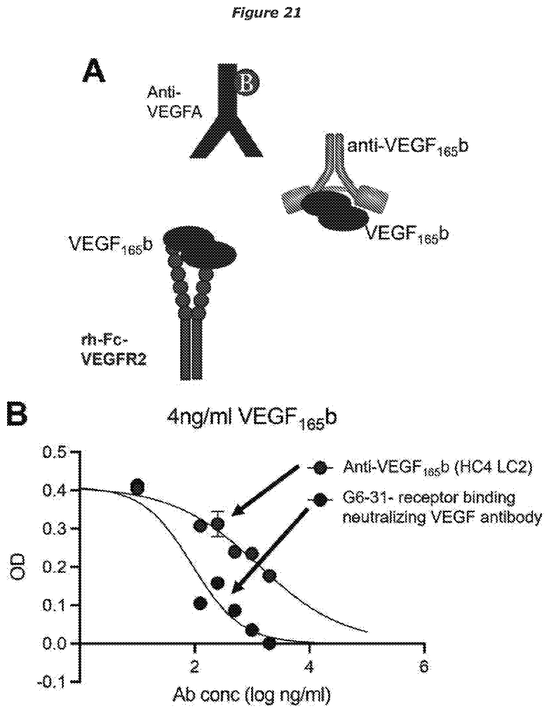

Furthermore, an exemplary antibody clone, HC4 LC2, was able to reduce the affinity of VEGF₁₆₅b to VEGF receptor 2 (VEGFR2), to a slightly lesser extent than the positive control of G6-31, an antibody that binds the receptor binding site (see FIG. 21B).

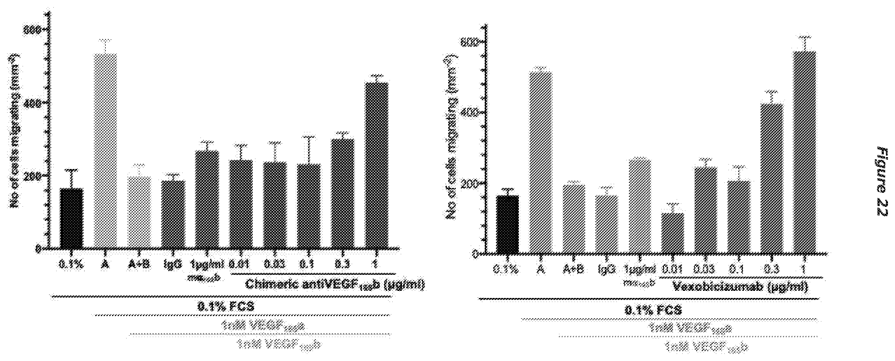

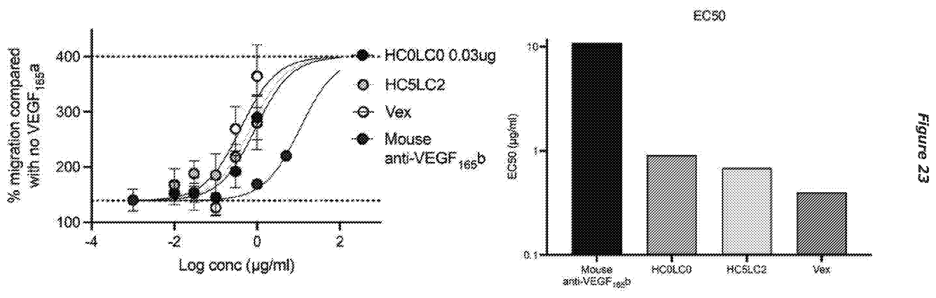

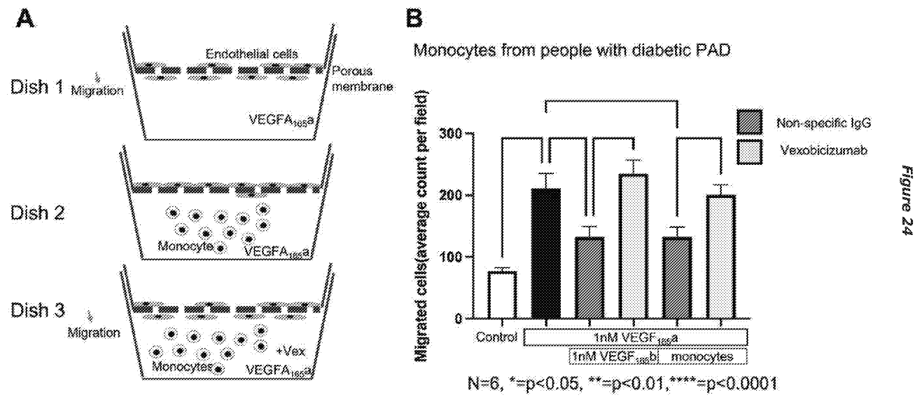

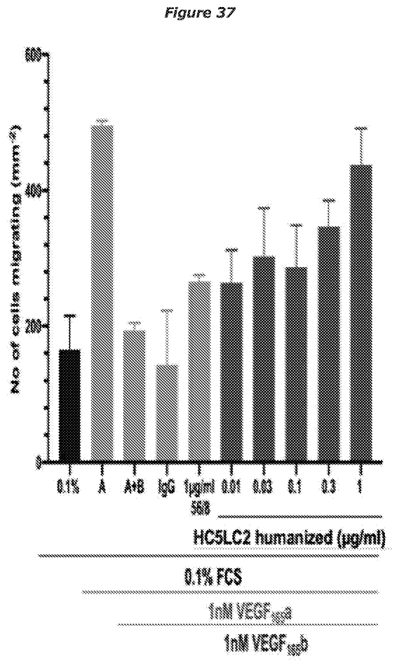

These two humanised variants were also able to block VEGF-A₁₆₅b mediated inhibition of cell migration in vitro more effectively than the parental mouse monoclonal and chimeric antibody (see FIGS. 22 and 37, and FIG. 23). Furthermore, the humanised variants were also capable of reversing diabetic peripheral arterial disease (PAD) human monocyte mediated inhibition of endothelial migration (see FIG. 24B).

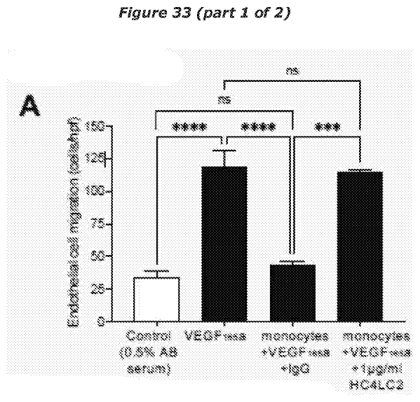

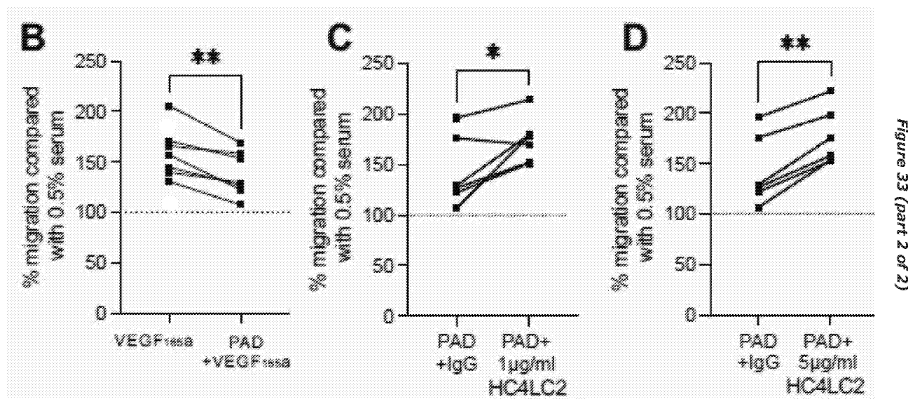

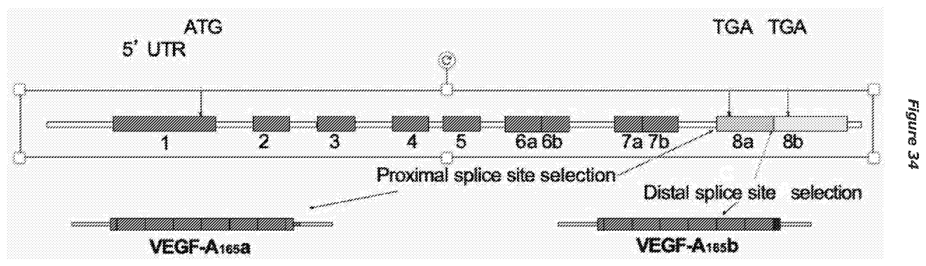

Further studies have shown that human monocyte-drive inhibition of the migration of endothelial cells (exemplified with human umbilical vein cells) across a porous membrane can be significantly reversed with 1 μg/ml HC4 LC2 (see FIG. 33A). Monocytes from 7 patients with peripheral vascular disease inhibited migration towards VEGF-A₁₆₅a (see FIG. 33B). Migration is increased at 1 μg/ml (see FIG. 33C) and 5 g/ml (see FIG. 33D) HC4 LC2.

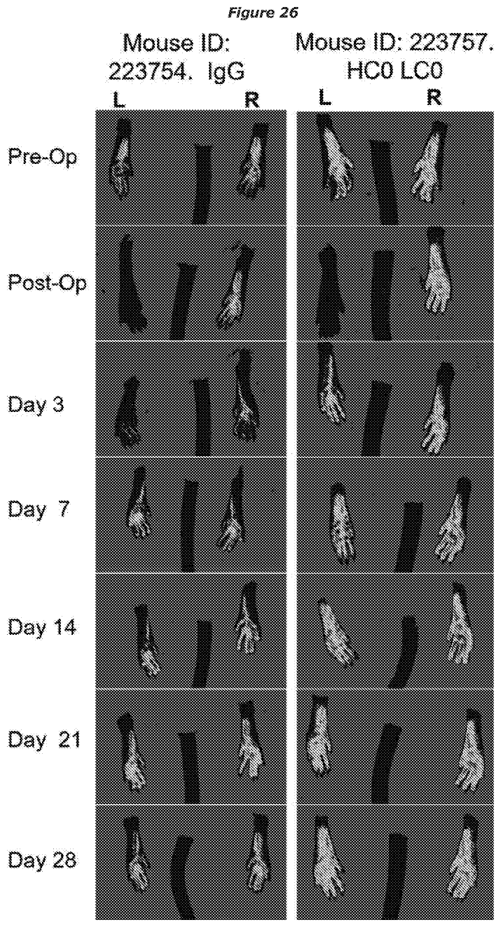

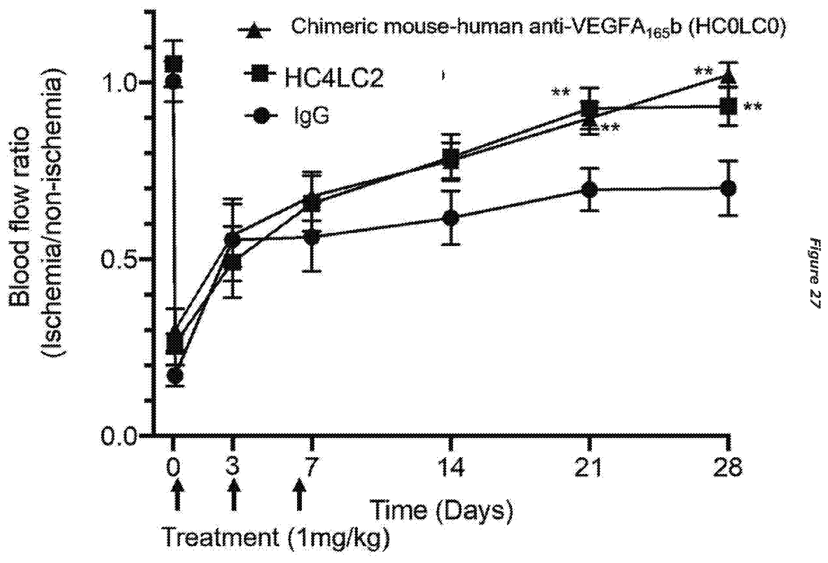

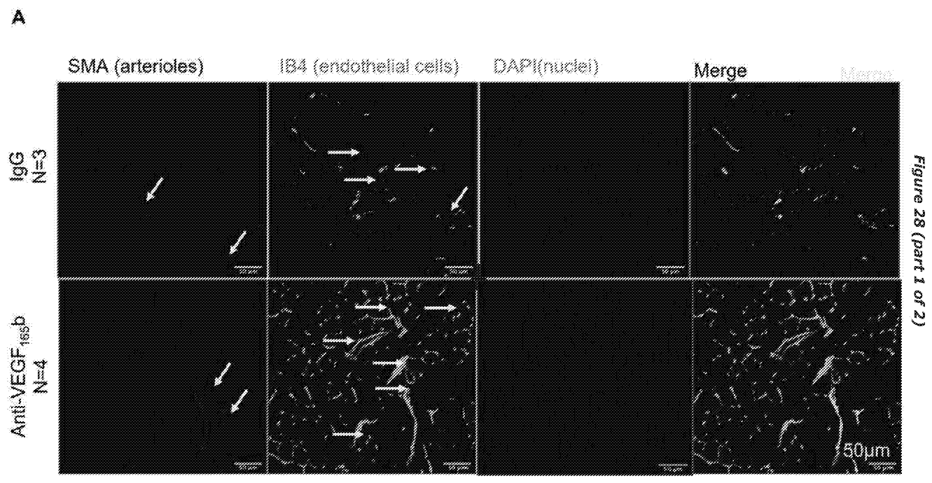

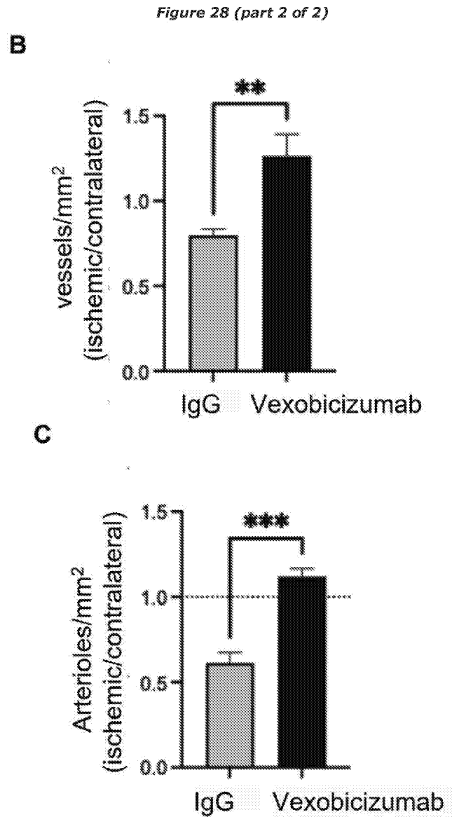

Blood flow recovery in IgG treated mice did not recover to normal after 28 days (day 14 66±6%, day 21 29±8.7%, day 28 75±8% of contralateral, N=6). This impaired revascularisation was rescued by humanised anti-VEGF-A₁₆₅b antibody (day 14, 78±5%, day 21, 90±4.6%, day 28 102±3.5%, N=7) see FIGS. 26 and 27. Furthermore, an improvement was observed in angiogenesis and arteriogenesis in HFHS obese mice (see FIG. 28A-C).

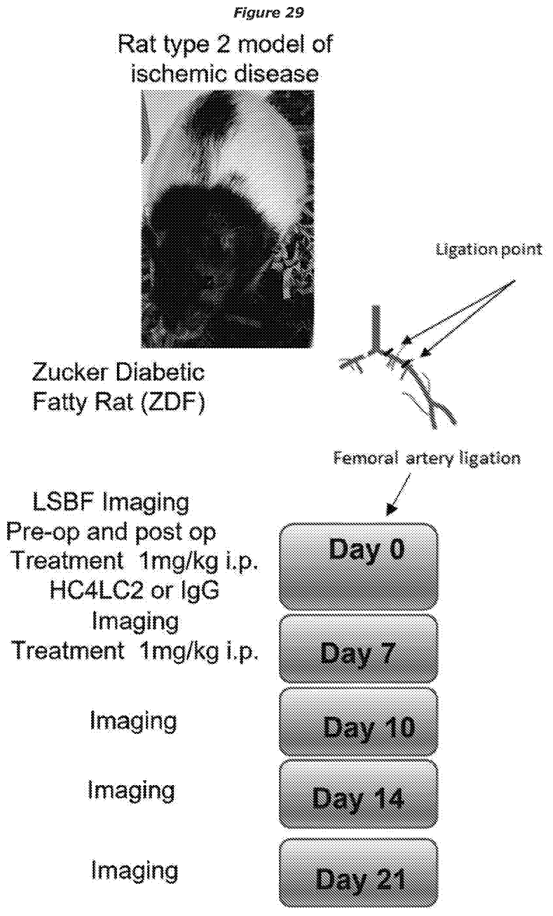

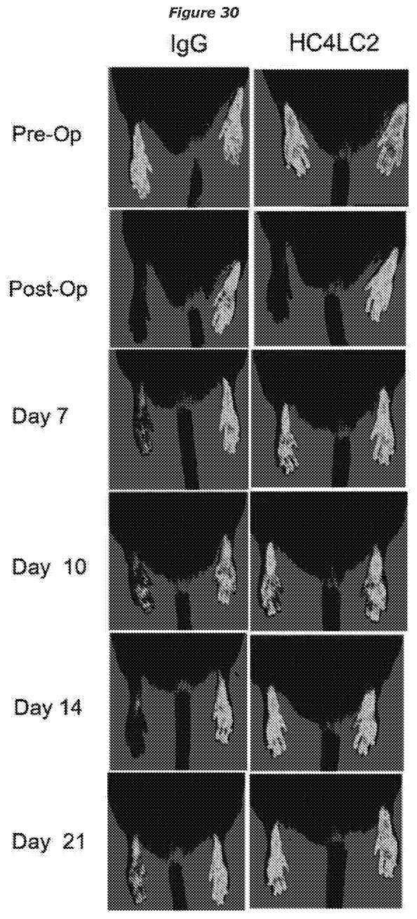

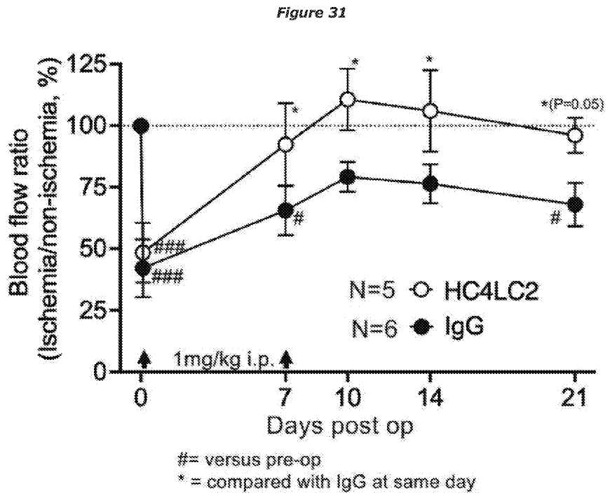

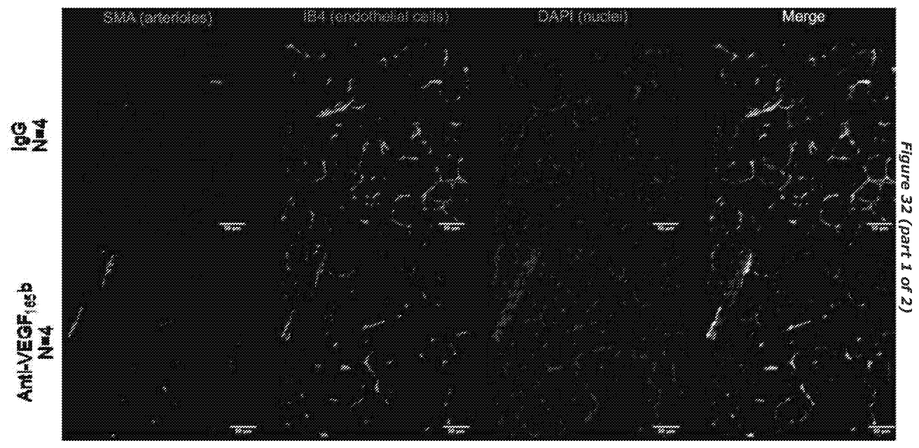

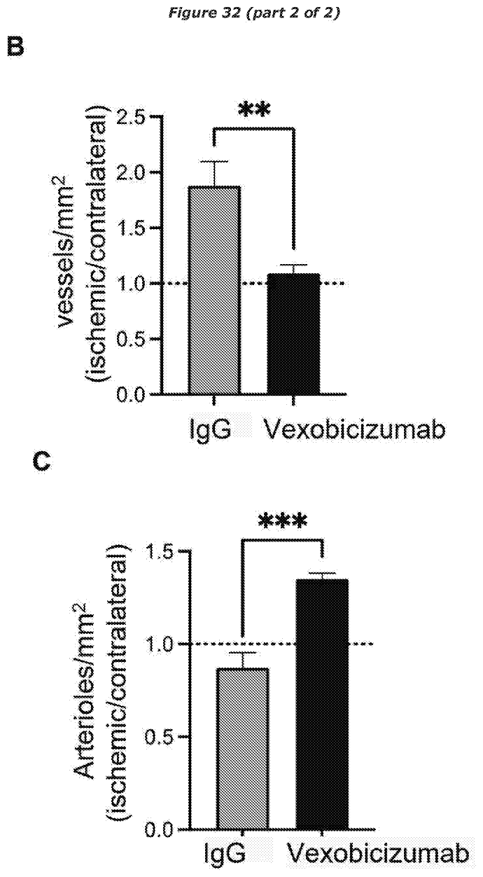

The effects were also seen in diabetic rats, the schematic of which can be seen in FIG. 29; with the resultant blood flow recovery in FIGS. 30 and 31; and the improvement to angiogenesis and arteriogenesis in FIG. 32A-C.

### Conclusion

Obesity results in impaired collateralisation and insufficient angiogenesis in PAD models. This is reversed with humanised anti-VEGF-A₁₆₅b suggesting that impaired revascularisation in patients with PAD could be treated with this antibody.

### Example 5: Humanisation of Anti-VEGF-A₁₆₅b mα165b

The sequences of the heavy chain (HC) variable region (mVH) and light chain (LC) variable region (mVL) of the parental mouse monoclonal antibody (Clone 58/8/31/1/13) are as follows:

Amino acid sequence of Clone 58/8/31/1/13 mVH
(SEQ ID NO: 15)
EVKLLESGGGLVQPGGSLKLSCAASGEDFSRYWMSWVRQAPG
KGLEWIGEIHPYSSTINYTPSVKDKFIISRDNAKNTLYLQMS
EVRSEDTALYYCARAFAYWGQGTLVTVSA
Amino acid sequence of Clone 58/8/31/1/13 mVL
(SEQ ID NO: 23)
DIVMTQTPLTLSVTIGQPASISCKSSQSLLDSDGKTYLNWLLQ
RPGQSPKRLIYLVSKLDSGVPDRETGSGSGTDFTLKISRVEAE
DLGVYYCWQGTHFPYTEGGGTKVEIKR

Blast analysis against the IMGT database of human antibody sequences⁶ reveals that the heavy chain CDRs (HCDRs—SEQ ID NOs: 1-3) from the mouse antibody (IGHV4-1*02-see Blast 1 below) have been grafted into a human IGHV3-11 framework (see Blast 2 below).

Blast 1 (HC1 VH region blast against IMGT database shows 82% identity with the closest mouse germline):

| >9323 AJ851868 Mus musculus IGHV4-1*02 VH | |
|---|---|
| Length = 98 | |
| Score = 179 bits (453) , Expect = 5e−47 | |
| Identities = 81/98 (82%), Positives = 94/98 (95%) | |
| Query: 1 | EVQLLESGGGLVKPGGSLRLSCAASGFDFSRYWMSWIRQAPGKGLEWVSEIHPYSSTINY 60 |
| | EV+LLESGGGLV+PGGSL+LSCAASGEDESRYWMSW+RQAPGKGLEW+ EI+P SSTINY |
| Sbjct: 1 | EVKLLESGGGLVQPGGSLKLSCAASGEDESRYWMSWVRQAPGKGLEWIGEINPDSSTINY 60 |
| Query: 61 | |
| | (SEQ ID NO: 83) |
| | TPSVKDRFTISRDNAKNSLYLQMNSLRAEDTAVYYCAR 98 |
| | (SEQ ID NO: 122) |
| | TPS+KD+F ISRDNAKN+LYLQM+ +R+EDTA+YYCAR |
| Sbjct: 61 | |
| | (SEQ ID NO: 84) |
| | TPSLKDKFIISRDNAKNTLYLQMSKVRSEDTALYYCAR 98 |

Blast 2 (HC1 VH region blast against IMGT database shows 86% identity with the closest human germline):

| >2883 X92287 Homo sapiens IGHV3-11*03 VH | |
|---|---|
| Length = 98 | |
| Score = 169 bits (429), Expect = 3e−44 | |
| Identities = 85/98 (86%), Positives = 87/98 (88%) | |
| Query: 1 | EVQLLESGGGLVKPGGSLRLSCAASGFDFSRYWMSWIRQAPGKGLEWVSEIHPYSSTINY 60 |
| | +VQLLESGGGLVKPGGSLRLSCAASGF FS Y+MSWIRQAPGKGLEWVS I SS NY |
| Sbjct: 1 | QVQLLESGGGLVKPGGSLRLSCAASGFTFSDYYMSWIRQAPGKGLEWVSYISSSSSYTNY 60 |
| Query: 61 | |
| | (SEQ ID NO: 83) |
| | TPSVKDRFTISRDNAKNSLYLQMNSLRAEDTAVYYCAR 98 |
| | (SEQ ID NO: 123) |
| | SVK RETISRDNAKNSLYLQMNSLRAEDTAVYYCAR |
| Sbjct: 61 | |
| | (SEQ ID NO: 85) |
| | ADSVKGRFTISRDNAKNSLYLQMNSLRAEDTAVYYCAR 98 |

The V kappa light of the parental antibody belongs to the family IGKV1-135*01. Blast analysis against the IMGT database⁷ of human antibody sequences reveals that the light chain CDRs from the mouse antibody have been grafted into a human IGKV2-30 framework (see Blast 3 below).

Blast 3 (LC1 VH region blast against IMGT database shows 91% identity with the closest human antibody VH sequence (IGKV2-30*02):

>11075 FM164408 Homo sapiens IGKV2-30*02 V-KAPPA
Length = 100
Score = 186 bits (472) , Expect = 3e−49
Identities = 91/100 (91%), Positives = 93/100 (93%)
Query: 1
DVVMTQSPLSLPVTLGQPASISCKSSQSLLDSDGKTYLNWFQQRPGQSPRRLIYLVSKLD 60
DVVMTQSPLSLPVTLGQPASISC+SSQSL+ SDG TYLNWFQQRPGQSPRRLIY VS D
Sbjct: 1
DVVMTQSPLSLPVTLGQPASISCRSSQSLVHSDGNTYLNWFQQRPGQSPRRLIYKVSNRD 60
Query: 61
(SEQ ID NO: 86)
SGVPDRESGSGSGTDFTLKISRVEAEDVGVYYCWQGTHEP 100
(SEQ ID NO: 124)
SGVPDRESGSGSGTDFTLKISRVEAEDVGVYYC QGTH P
Sbjct: 61
(SEQ ID NO: 87)
SGVPDRESGSGSGTDFTLKISRVEAEDVGVYYCMQGTHWP 100

### Humanization Check

The humanized variants were checked to determine whether they had been humanized in accordance with WHO's definition of humanized antibodies¹¹; the variable domain of a humanized chain has a V region amino acid sequence which, analysed as a whole, is closer to human than to other species (assessed using the Immunogenetics Information System® (IMGT®) DomainGapAlign tool)¹². Results are shown in Table 5. All variants can be considered humanized. The approved INN name for the therapeutic candidate is Vexobicizumab.

TABLE 5
WHO's assigned antibody INN for
the murine and humanized variants.

| | | Gene | Smith- | % | | |
|---|---|---|---|---|---|---|
| Sequence | | and | Waterman | Iden- | Over- | WHO INN |
| name | Species | allele | score | tity | lap | designation |
| VH0 | Mus | IGHV4- | 636 | 95.9 | 98 | Mouse |
| | musculus | 1*02 | | | | |
| VH1 | Homo | IGHV3- | 552 | 86.7 | 98 | Humanized |
| | sapiens | 11*03 | | | | |
| VH2 | Homo | IGHV3- | 544 | 83.7 | 98 | Humanized |
| | sapiens | 7*01 | | | | |
| VH3# | Mus | IGHV4- | 591 | 85.7 | 98 | Mouse |
| | musculus | 1*02 | | | | |
| VH3 | Homo | IGHV3- | 550 | 85.7 | 98 | Humanized |
| | sapiens | 48*02 | | | | |
| VH4 | Homo | IGHV3- | 554 | 85.7 | 98 | Humanized |
| | sapiens | 7*01 | | | | |
| VH5 | Homo | IGHV3- | 552 | 87.9 | 98 | Humanized |
| | sapiens | 48*01 | | | | |
| VL0 | Mus | IGKV1- | 659 | 98.0 | 100 | Mouse |
| | musculus | 135*01 | | | | |
| VL1 | Homo | IGKV2- | 611 | 91.0 | 100 | Humanized |
| | sapiens | 30*02 | | | | |
| VL2# | Mus | IGKV1- | 624 | 90.0 | 100 | Mouse |
| | musculus | 135*01 | | | | |
| VL2 | Homo | IGKV2- | 595 | 90.0 | 100 | Humanized |
| | sapiens | 30*02 | | | | |
| VL3 | Homo | IGKV2- | 585 | 92.0 | 100 | Humanized |
| | sapiens | 29*02 | | | | |
| VL4** | Macaca | IGKV2 | 580 | 87.8 | 98 | Macaque |
| | mulatta | S8*01 | | | | |
| VL4 | Macaca | IGKV2 | 580 | 87.8 | 98 | Macaque |
| | mulatta | S9*01 | | | | |
| VL4 | Homo | IGKV2- | 578 | 87.0 | 100 | Humanized |
| | sapiens | 24*01 | | | | |
| VL5 | Homo | IGKV2- | 591 | 91.0 | 100 | Humanized |
| | sapiens | 29*02 | | | | |

#Equally as close to human as mouse
**Equally as close to human as macaque

### Characterization of Antibodies

Twenty-five humanized full length antibody variants, designated HCX LCX (X=1-5) were generated by combining each variant light chain (LC1-5) with each variant heavy chain (HCl-5). In addition, a chimeric version of the antibody was made (designated HC0 LC0) by grafting the full-length mouse VH and VL domains onto human IgG1 heavy chain constant region (CH) and human kappa LC constant region (CL) respectively. The antibodies were transiently expressed in CHO cells, purified from culture supernatants by Protein A affinity chromatography and dialysed/buffer exchanged into PBS for in vitro testing.

The yields of antibody (in mg/L) were comparable or higher than for the chimeric antibody HC0 LC0 except for HC1 LC1 and HC3 LC4. The yield of antibodies expressed with LC3, LC4 or LC5 light chains were overall expressed at about 16% lower than for the LC1 and LC2 antibodies (n=1). All antibodies seem intact by SDS-PAGE with some degradation products or contaminants observed for HC1 LC2, HC1 LC4, HC3 LC4, HC4 LC2 and HC4 LC5 on non-reducing gels.

Kinetic analysis of each of the antibodies was performed by biolayer interferometry (BLI) using the Fortebio Octet system. Antibodies were captured on anti-human Fc biosensors then incubated with commercially available human VEGF-A₁₆₅b purchased from Sino Biological. Capture levels of each antibody to the Fc biosensor were comparable. Kinetic analysis showed that all HCX LC1 and HCX LC2 antibodies exhibited binding characteristics similar to the chimeric control antibody HC0 LC0. Using a 1:1 binding model, it was not possible to determine ka and kd values for the other antibodies. Under the conditions of the assay, the affinity of HC0 LC0 (0.3 nM) binding to VEGF-A₁₆₅b was 2× higher than for HC4 LC2 and HC5 LC2 (0.6 nM) and 3× higher than HC4 LC1 (0.9 nM). Antibodies which had affinities >1 nM were HC1 LC1 (5.0 nM), HC1 LC2 (2.1 nM), HC2 LC1 (4.4 nM) HC2 LC2 (3.6 nM), HC3 LC1 (1.5 nM), HC3 LC2 (1.17 nM) and HC5 LC1 (3.2 nM).

Scale up of HC0 LC0, HC4 LC2 and HC5 LC2 was subsequently performed. Antibodies were transiently expressed in CHO cells and purified by Protein A affinity chromatography from culture supernatants. Purified mAbs were buffer exchanged into PBS and analysed by SDS-PAGE and size exclusion chromatography. By non-reducing SDS-PAGE, purity was estimated at >95%. By SEC (n=1), a single peak corresponding to the monomeric fraction (>98%) was detected for all antibodies with a similar retention time. Endotoxin content was measured at <1.0 EU/mg satisfying the criteria for in vivo administration.

### In Vitro and In Vivo Testing

In an endothelial cell migration assay, when tested at 1 μg/ml, the rank order potency of neutralisation of the anti-angiogenic effect of VEGF-A₁₆₅b was HC4 LC2 >HC5 LC2>HC0 LC0>mAb 58/6. The IC50 values for inhibition of VEGF-A₁₆₅b (% activity) were 442 ng/ml, 137 ng/ml, and 114 ng/ml for HC0 LC0, HC4 LC2, and HC5 LC2 respectively.

The chimeric antibody HC0 LC0 is able to reverse obesity-dependent, impaired revascularisation in a mouse femoral artery ligation ischemia model for up to 30 days when administered i.p. at 1 mg/kg on Day 1, Day 3 and Day 7 after ischemia. The humanized antibodies have not yet been tested in this model. Proof of concept has also recently been demonstrated by Navarro et al¹⁴, In vivo blockade of VEGF-A₁₆₅b increased neoangiogenesis, reduced infarct size, and enhanced systolic function in reperfused, but not in non-reperfused, myocardial infarction models.

### REFERENCES

1. Cébe-Suarez, S., Zehnder-Fjällman, A. & Ballmer-Hofer, K. The role of VEGF receptors in angiogenesis; complex partnerships. Cell. Mol. Life Sci. 63, 601-615 (2006)
2. Kikuchi, R. et al. An antiangiogenic isoform of VEGF-A contributes to impaired vascularization in peripheral artery disease. Nat. Med. 20, 1464-1471 (2014).
3. Carter, J. G. et al. The carboxyl terminus of VEGF-A is a potential target for anti-angiogenic therapy. Angiogenesis 18, 23-30 (2015).
4. Woolard, J. et al. VEGF165b, an inhibitory vascular endothelial growth factor splice variant: mechanism of action, in vivo effect on angiogenesis and endogenous protein expression. Cancer Res. 64, 7822-7835 (2004).
5. Beck, A. & Liu, H. Macro- and Micro-Heterogeneity of Natural and Recombinant IgG Antibodies. (2019). doi:10.3390/antib8010018
6. Lefranc, M.-P. IMGT, the international ImMunoGeneTics information system, http://imgt.cines.fr. Novartis Found. Symp. 254, 126-142,216-222,250-252 (2003).
7. Lefranc, M.-P. et al. IMGT unique numbering for immunoglobulin and T cell receptor variable domains and Ig superfamily V-like domains. Dev. Comp. Immunol. 27, 55-77 (2003).
8. Jefferis, R. Recombinant antibody therapeutics: the impact of glycosylation on mechanisms of action. Trends Pharmacol. Sci. 30, 356-362 (2009).
9. van de Bovenkamp, F. S. et al. Variable Domain N-Linked Glycans Acquired During Antigen-Specific Immune Responses Can Contribute to Immunoglobulin G Antibody Stability. Front. Immunol. 9, 740 (2018),
10. Jarasch, A. et al. Developability assessment during the selection of novel therapeutic antibodies. J. Pharm. Sci. 104, 1885-1898 (2015).
11. Jones, T. D. et al. The INNs and outs of antibody nonproprietary names. MAbs 8, 1-9 (2016).
12. Ehrenmann, F., Kaas, Q, & Lefranc, M.-P. IMGT/3Dstructure-DB and IMGT/DomainGapAlign: a database and a tool for immunoglobulins or antibodies, T cell receptors, MHC, IgSF and MhcSF. Nucleic Acids Res. 38, D301-7 (2010).
13. Paul, S. et al. Development and validation of a broad scheme for prediction of HLA class II restricted T cell epitopes. J. Immunol. Methods 422, 28-34 (2015).
14. Ríos-Navarro, C. et al. Role of antiangiogenic VEGF-A(165)b in angiogenesis and systolic function after reperfused myocardial infarction. Rev. Esp. Cardiol. (Engl. Ed). 74, 131-139 (2021).
15. Haberger, M. et al. Functional assessment of antibody oxidation by native mass spectrometry, MAbs 7, 891-900 (2015).
16. Brader, M. L. et al. Examination of thermal unfolding and aggregation profiles of a series of developable therapeutic monoclonal antibodies. Mol. Pharm. 12, 1005-1017 (2015).
17. Igawa, T. et al. Engineering the variable region of therapeutic IgG antibodies. MAbs 3, 243-252 (2011).
18. Liu, H., Saxena, A., Sidhu, S. S. & Wu, D. Fc Engineering for Developing Therapeutic Bispecific Antibodies and Novel Scaffolds. Front Immunol 8, 38 (2017).
19. Temel, D. B., Landsman, P. & Brader, M. L. Orthogonal Methods for Characterizing the Unfolding of Therapeutic Monoclonal Antibodies: Differential Scanning calorimetry, Isothermal Chemical Denaturation, and Intrinsic Fluorescence with Concomitant Static Light Scattering. Methods Enzymol. 567, 359-389 (2016).
20. Chai, Q., Shih, J., Weldon, C., Phan, S. & Jones, B. E. Development of a high-throughput solubility screening assay for use in antibody discovery. MAbs 11, 747-756 (2019).
21. Bhirde, A. et al. High Performance Size Exclusion Chromatography and High-Throughput Dynamic Light Scattering as Orthogonal Methods to Screen for Aggregation and Stability of Monoclonal Antibody Drug Products. J. Pharm. Sci. 109, 3330-3339 (2020).
22. Avery, L. B. et al. Establishing in vitro in vivo correlations to screen monoclonal antibodies for physicochemical properties related to favorable human pharmacokinetics. MAbs 10, 244-255 (2018).
23. Schlothauer, T. et al. Analytical FcRn affinity chromatography for functional characterization of monoclonal antibodies. MAbs 5, 576-586 (2013).
24. Kraft, T. E. et al. Heparin chromatography as an in vitro predictor for antibody clearance rate through pinocytosis. MAbs 12, 1683432 (2020).
25. Nugent, P., Duncan, J. N. & Colagiovanni, D. B. The Preparation of a Preclinical Dossier to Support an Investigational New Drug (IND) Application and First-in-Human Clinical Trial. In A Comprehensive Guide to Toxicology in Preclinical Drug Development 309-334 (Elsevier Inc., 2013). doi: 10.1016/B978-0-12-387815-1.00012-5
26. Avery, L. B. et al. Utility of a human FcRn transgenic mouse model in drug discovery for early assessment and prediction of human pharmacokinetics of monoclonal antibodies. MAbs 8, 1064-1078 (2016).
27. Kuppuswamy et al., Targeting Anti-Angiogenic VEGF165b-VEGFR1 Signaling Promotes Nitric Oxide Independent Therapeutic Angiogenesis in Preclinical Peripheral Artery Disease Models, Cells 2022, 11, 2676.

### Example 6: Sequences for Site Directed Mutagenesis

### Overview

A chimeric version, HC0 LC0, an anti-VEGF-A₁₆₅b mAb was generated containing the VH and VL of the original mouse antibody (Ab 1126). Twenty-five humanized variants were generated of which HC4 LC2 and HC5 LC2 had binding affinities within 2× of the chimeric antibody. A number of sequence liabilities were detected in both the VH and VL of the humanized variants. The next step of development of the humanised antibody was to attempt to remove potential sequence liabilities through site-directed mutagenesis of the VH and VL regions, without significant loss in affinity or potency compared to the chimeric antibody.

In addition, mutations in the Fc region of IgG1 will also be tested: LALA=L234A L235A*, to remove Fc receptor binding, and YTE=M252Y S254T Y256* for half-life extension. The chimeric antibody HC0 LC0 was tested alongside YTE only and LALA+YTE variants to ensure that changes in the Fc have no impact on antibody binding to target. Sequence liability mutants were tested in wild type IgG1 format in the first instance to identify mutations which are tolerated (no loss of binding or functional activity) before making a further set of antibody variants in which HC and LC mutations will be combined. (*canonical numbering.)

### List of Expressed Antibodies:

| Heavy chain | Light chain |
|---|---|
| HC0 YTE | LC0 |
| HC0 LALA +YTE | LC0 |
| HC4 YTE | LC2 |
| HC5 YTE | LC2 |
| HC4 N59Q | LC1 |
| HC5 N59Q | LC2 |
| HC4 N59Q | LC2 |
| HC4 T61A | LC2 |
| HC5 T61A | LC2 |
| HC4 | LC1 S32A |
| HC4 | LC2 S32A |
| HC5 | LC2 S32A |
| HC4 | LC1 D33E |
| HC4 | LC2 D33E |
| HC5 | LC2 D33E |
| HC4 | LC1 G34A |
| HC4 | LC2 G34A |
| HC5 | LC2 G34A |
| HC0 | LC0 |
| HC4 | LC1 |
| HC4 | LC2 |
| HC5 | LC2 |

Antibodies were cloned, expressed and purified in mg scale. Parameters measured include: yield, SDS-PAGE (reducing and non-reducing), analytical SEC, binding affinity to VEGF-A₁₆₅b compared with parental antibodies (HC0 LC0, HC4 LC2, HC5 LC2 and HC4 LC1).

Antibody Sequences without Signal Peptide

Residues to be mutated are shown in underlined (Fc domain) or double-underlined (variable domain).

> HCQ
(SEQ ID NO: 99)
EVKLLESGGGLVQPGGSLKLSCAASGEDESRYWMSWVRQAPGKGLEWIGEIHPYSSTINYTPSVKDKFI
ISRDNAKNTLYLQMSEVRSEDTALYYCARAFAYWGQGTLVTVSAASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVTCVVVDVSHEDPEVKENWYVD
GVEVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGK
> HCQ YTE
(SEQ ID NO: 100)
EVKLLESGGGLVQPGGSLKLSCAASGEDFSRYWMSWVRQAPGKGLEWIGEIHPYSSTINYTPSVKDKFI
ISRDNAKNTLYLQMSEVRSEDTALYYCARAFAYWGQGTLVTVSAASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLYITREPEVTCVVVDVSHEDPEVKENWYVD
GVEVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGK
>HCQ LALA YTE
(SEQ ID NO: 101)
EVKLLESGGGLVQPGGSLKLSCAASGFDFSRYWMSWVRQAPGKGLEWIGEIHPYSSTINYTPSVKDKFI
ISRDNAKNTLYLQMSEVRSEDTALYYCARAFAYWGQGTLVTVSAASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKV
DKKVEPKSCDKTHTCPPCPAPEAAGGPSVFLFPPKPKDTLYITREPEVTCVVVDVSHEDPEVKENWYVD
GVEVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGK
Heavy chain mutants:
>HC4 YTE
(SEQ ID NO: 102)
EVQLVESGGGLVQPGGSLRLSCAASGEDESRYWMSWVRQAPGKGLEWVAEIHPYSSTINYTPSVKDRET
ISRDNAKNSLYLQMNSLRSEDTAVYYCARAFAYWGQGTLVTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLYITREPEVTCVVVDVSHEDPEVKENWYVD
GVEVHNAKTKPREEQYNSTYRVVSVLTVLHQDWINGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGK
>HC5 YTE
(SEQ ID NO: 103)
EVQLVESGGGLVQPGGSLRLSCAASGEDESRYWMSWVRQAPGKGLEWVSEIHPYSSTINYTPSVKDRET
ISRDNAKNSLYLQMNSLRAEDTAVYYCARAFAYWGQGTLVTVSAASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLYITREPEVTCVVVDVSHEDPEVKENWYVD
GVEVHNAKTKPREEQYNSTYRVVSVLTVLHQDWINGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGK
>HC4 N59Q
(SEQ ID NO: 104)
EVQLVESGGGLVQPGGSLRLSCAASGEDESRYWMSWVRQAPGKGLEWVAEIHPYSSTIQYTPSVKDRFT
ISRDNAKNSLYLQMNSLRSEDTAVYYCARAFAYWGQGTLVTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVTCVVVDVSHEDPEVKENWYVD
GVEVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGK
>HC5 N59Q
(SEQ ID NO: 105)
EVQLVESGGGLVQPGGSLRLSCAASGEDESRYWMSWVRQAPGKGLEWVSEIHPYSSTIQYTPSVKDRET
ISRDNAKNSLYLQMNSLRAEDTAVYYCARAFAYWGQGTLVTVSAASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVTCVVVDVSHEDPEVKENWYVD
GVEVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGK
>HC4 T61A
(SEQ ID NO: 106)
EVQLVESGGGLVQPGGSLRLSCAASGEDFSRYWMSWVRQAPGKGLEWVAEIHPYSSTINYAPSVKDRET
ISRDNAKNSLYLQMNSLRSEDTAVYYCARAFAYWGQGTLVTVSSASTKGPSVFPLAPSSKSTSGGTAAL
GCLVKDYFPEPVTVSWNSGALTSGVHTFPAVLQSSGLYSLSSVVTVPSSSIGTQTYICNVNHKPSNTKV
DKKVEPKSCDKTHTCPPCPAPELLGGPSVFLFPPKPKDTLMISRTPEVTCVVVDVSHEDPEVKENWYVD
GVEVHNAKTKPREEQYNSTYRVVSVLTVLHQDWINGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGK
>HC5 T61A
(SEQ ID NO: 107)
EVQLVESGGGLVQPGGSLRLSCAASGFDFSRYWMSWVRQAPGKGLEWVSEIHPYSSTINYAPSVKDRET
ISRDNAKNSLYLQMNSLRAEDTAVYYCARAFAYWGQGTLVTVSAASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTIMISRTPEVTCVVVDVSHEDPEVKENWYVD
GVEVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGK
Light chain mutants:
>LCQ
(SEQ ID NO: 108)
DIVMTQTPLTLSVTIGQPASISCKSSQSLLDSDGKTYLNWLLQRPGQSPKRLIYLVSKLDSGVPDRETG
SGSGTDFTLKISRVEAEDLGVYYCWQGTHFPYTFGGGTKVEIKRTVAAPSVFIFPPSDEQLKSGTASVV
CLLNNFYPREAKVQWKVDNALQSGNSQESVTEQDSKDSTYSLSSTLTLSKADYEKHKVYACEVTHQGLS
SPVTKSENRGEC
>LC1
(SEQ ID NO: 109)
DVVMTQSPLSLPVTLGQPASISCKSSQSLLDSDGKTYINWFQQRPGQSPRRLIYLVSKLDSGVPDRESG
SGSGTDFTLKISRVEAEDVGVYYCWQGTHFPYTFGGGTKVEIKRTVAAPSVFIFPPSDEQLKSGTASVV
CLLNNFYPREAKVQWKVDNALQSGNSQESVTEQDSKDSTYSLSSTLTLSKADYEKHKVYACEVTHQGLS
SPVTKSENRGEC
>LC2
(SEQ ID NO: 110)
DVVMTQSPLSLPVTLGQPASISCRSSQSLLDSDGKTYLNWFLQRPGQSPRRLIYLVSKLDSGVPDRESG
SGSGTDFTLKISRVEAEDVGVYYCWQGTHEPYTFGQGTKVEIKRTVAAPSVFIFPPSDEQLKSGTASVV
CLLNNFYPREAKVQWKVDNALQSGNSQESVTEQDSKDSTYSLSSTLTLSKADYEKHKVYACEVTHQGLS
SPVTKSENRGEC
>LC1 S32A
(SEQ ID NO: 111)
DVVMTQSPLSLPVTLGQPASISCKSSQSLLDADGKTYLNWFQQRPGQSPRRLIYLVSKLDSGVPDRESG
SGSGTDFTLKISRVEAEDVGVYYCWQGTHFPYTFGGGTKVEIKRTVAAPSVFIFPPSDEQLKSGTASVV
CLLNNFYPREAKVQWKVDNALQSGNSQESVTEQDSKDSTYSLSSTLTLSKADYEKHKVYACEVTHQGLS
SPVTKSENRGEC
>LC2 S32A
(SEQ ID NO: 112)
DVVMTQSPLSLPVTLGQPASISCRSSQSLLDADGKTYLNWFLQRPGQSPRRLIYLVSKLDSGVPDRFSG
SGSGTDFTLKISRVEAEDVGVYYCWQGTHEPYTFGQGTKVEIKRTVAAPSVFIFPPSDEQLKSGTASVV
CLLNNFYPREAKVQWKVDNALQSGNSQESVTEQDSKDSTYSLSSTLTLSKADYEKHKVYACEVTHQGLS
SPVTKSFNRGEC
>LC1 D33E
(SEQ ID NO: 113)
DVVMTQSPLSLPVTLGQPASISCKSSQSLLDSEGKTYLNWFQQRPGQSPRRLIYLVSKLDSGVPDRESG
SGSGTDFTLKISRVEAEDVGVYYCWQGTHFPYTFGGGTKVEIKRTVAAPSVFIFPPSDEQLKSGTASVV
CLLNNFYPREAKVQWKVDNALQSGNSQESVTEQDSKDSTYSLSSTLTLSKADYEKHKVYACEVTHQGLS
SPVTKSENRGEC
>LC2 D33E
(SEQ ID NO: 114)
DVVMTQSPLSLPVTLGQPASISCRSSQSLLDSEGKTYLNWFLQRPGQSPRRLIYLVSKLDSGVPDRESG
SGSGTDFTLKISRVEAEDVGVYYCWQGTHEPYTFGQGTKVEIKRTVAAPSVFIFPPSDEQLKSGTASVV
CLENNFYPREAKVQWKVDNALQSGNSQESVTEQDSKDSTYSLSSTLTLSKADYEKHKVYACEVTHQGLS
SPVTKSFNRGEC
>LC1 G34A
(SEQ ID NO: 115)
DVVMTQSPLSLPVTLGQPASISCKSSQSLLDSDAKTYLNWFQQRPGQSPRRLIYLVSKLDSGVPDRESG
SGSGTDFTLKISRVEAEDVGVYYCWQGTHFPYTFGGGTKVEIKRTVAAPSVFIFPPSDEQLKSGTASVV
CLLNNFYPREAKVQWKVDNALQSGNSQESVTEQDSKDSTYSLSSTLTLSKADYEKHKVYACEVTHQGLS
SPVTKSENRGEC
>LC2 G34A
(SEQ ID NO: 116)
DVVMTQSPLSLPVTLGQPASISCRSSQSLLDSDAKTYLNWFLQRPGQSPRRLIYLVSKLDSGVPDRESG
SGSGTDFTLKISRVEAEDVGVYYCWQGTHEPYTEGQGTKVEIKRTVAAPSVFIFPPSDEQLKSGTASVV
CLLNNFYPREAKVQWKVDNALQSGNSQESVTEQDSKDSTYSLSSTLTLSKADYEKHKVYACEVTHQGLS
SPVTKSENRGEC
>LC1 D60E
(SEQ ID NO: 117)
DVVMTQSPLSLPVTLGQPASISCKSSQSLLDSDGKTYLNWFQQRPGQSPRRLIYLVSKLESGVPDRESG
SGSGTDFTLKISRVEAEDVGVYYCWQGTHFPYTFGGGTKVEIKRTVAAPSVFIFPPSDEQLKSGTASVV
CLLNNFYPREAKVQWKVDNALQSGNSQESVTEQDSKDSTYSLSSTLTLSKADYEKHKVYACEVTHQGLS
SPVTKS ENRGEC
>LC2 D60E
(SEQ ID NO: 118)
DVVMTQSPLSLPVTLGQPASISCRSSQSLLDSDGKTYLNWFLQRPGQSPRRLIYLVSKLESGVPDRFSG
SGSGTDFTLKISRVEAEDVGVYYCWQGTHEPYTEGQGTKVEIKRTVAAPSVFIFPPSDEQLKSGTASVV
CLLNNFYPREAKVQWKVDNALQSGNSQESVTEQDSKDSTYSLSSTLTLSKADYEKHKVYACEVTHQGLS
SPVTKSENRGEC

### Example 7: Anti-VEGF₁₆₅b Antibodies Inhibit Binding of VEGF₁₆₅b to VEGFR2

### Method

A 96-well clear microplate (high sensitivity thermo immulon or costar 9018) was coated with 100 ml per well of 1 μg/μl rh-Fc-VEGFR2 (357-KD-050/CF, R&D Systems) overnight, shaking at room temp. Anti-VEGF₁₆₅b antibody/VEGF₁₆₅b mixtures was made up with 4 ng/ml VEGF-A₁₆₅b and increasing concentrations of each antibody and incubate for 2 hours at 4 °C. The VEGFR2 coated plate was washed with 0.05% tween20 in PBS (PBST) three times. The plate was blocked with Suberblock buffer and washed 3 times. 100 μl of the anti-VEGF₁₆₅b antibody/VEGF₁₆₅b mixture was added and incubated for 1 hour followed by a 3×wash. 100 μl Biotinylated anti-VEGF-A (BAF293, R&D Systems) in 1% BSA/PBS was added and incubated for 2 hours at room temp. This was washed 3 times followed by incubation with 100 μl streptavidin-HRP in filtered 1% BSA/PBS. The plate was covered with foil and Incubated shaking for 30 minutes at room temperature. After 3 washes tetramethylbenzidine (TMB) substrate was added until leave colour developed. The reaction was stopped with 50 μl of 1M HCL, Optical density was measured using a microplate reader set to 450 nm with a 620 nm reference.

### Results

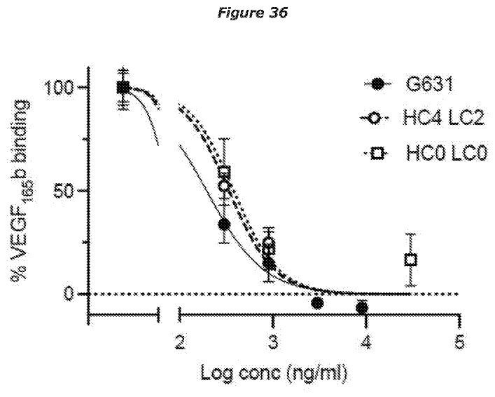

To determine the neutralising effect of the antibodies VEGFR2 was bound to an ELISA plate and the anti-VEGF-A₁₆₅b antibodies mixed with rhVEGF-A₁₆₅b at increasing concentrations of VEGF-A₁₆₅b antibody. Humanised (HC4LC2) and chimeric (HC0LC0) anti-VEGF₁₆₅b antibodies were both able to significantly reduce the affinity of VEGF₁₆₅b to VEGF receptor 2 (VEGFR2), again, to a slightly lesser extent than the positive control of G6-31 (a pan VEGF antibody that binds to the receptor binding site of VEGF-A) (FIG. 36). These results support the findings discussed in Example 4.

### Example 8: Additional Sequences for Site Directed Mutagenesis

### Background

Inducing the complement cascade has been associated with antibody injection site adverse reactions and off-target cell lysis, also known as complement dependent cellular cytotoxicity (CDC). Interaction of the Fc domain of an antibody which recognises a target on the cell surface, can lead to binding of complement factor C1q, which in turn can activate the complement cascade and mediate complement dependent cytotoxicity (CDC) resulting in destruction of the cell to which the antibody is bound. C1q binding is the first step in the initiation of the complement cascade, C1q forms a complex with the serine proteases C1r and C1s to form the C1 complex. C1q is capable of binding six antibodies, although binding to two IgGs is sufficient to activate the complement cascade. Therefore, eliminating C1q binding to Fc—the initial event in the activation of antibody-dependent complement cytotoxicity—can be achieved through site-directed mutagenesis of specific residues in the Fc domain of antibodies.

### Context

Vexobicizumab is a humanised IgG1 antibody. IgG1 is known to activate complement. However, the activation requires clustering of the antibody on the cell surface via interaction with its specific target (receptor) expressed on the cell surface. In theory, Vexobicizumab should not fix complement as its target, VEGF-A₁₆₅b, is a soluble molecule. However, non-specific binding of VEGF-A may occur through binding to heparin sulphate proteoglycans (HSPGs) expressed on the surface of non-immune cells, e.g. endothelial cells, epithelial cells and fibroblasts. Unlike the pro-angiogenic form of VEGF-A (VEGF-A₁₆₅a), the VEGF-A₁₆₅b splice variant has been reported (data not shown) to have low or no binding to heparin (despite the fact that it is predicted to contain a heparin binding domain), so, therefore, it should not bind to heparin sulphate proteoglycans on the cell surface, making it susceptible to complement mediated lysis. On the other hand, it is unknown if binding of VEGF-A₁₆₅b to its receptor VEGFR2 mediates internalization of the receptor, thus it is possible that the receptor-ligand antibody complex (transiently) present on the cell surface may elicit Fc-mediated complement activation.

Mutations may be introduced to reduce risk of possible cytopathic off-target effects on specialized epithelial cells in eye and kidney due to presence of surface bound VEGF-A₁₆₅b. These cells are on the far side of the semi-permeable barriers which normally exclude antibody-sized molecules so it may not be a problem. The mutations may contain single amino acid mutations in the Fc domain that have been reported to abrogate Ciq binding and thus limit complement activation, without impacting binding to the neonatal FC receptor (FcRn).

### Sequences

The following single amino acid mutations in human IgG1 heavy chain of HC4 LC2 are proposed:
D270A (Lazar G A, Dang W, Karki S, Vafa O, Peng J S, Hyun L, et al. Engineered antibody Fc variants with enhanced effector function. Proc Natl Acad Sci USA. (2006) 103:4005-10. doi: 10.1073/pnas.0508123103).
P329A (Idusogie E E, Presta L G, Gazzano-Santoro H, Totpal K, Wong P Y, Ultsch M, et al. Mapping of the C1q binding site on rituxan, a chimeric antibody with a human IgG1 Fc. J Immunol. (2000) 164:4178-84. doi: 10.4049/jimmunol.164.8.4178).
P331S (Xu Y, Oomen R, Klein M H. Residue at position 331 in the IgG1 and IgG4 CH2 domains contributes to their differential ability to bind and activate complement. J Biol Chem. (1994) 269:3469-74).

Key:
Bold highlighting = VH
Underlined = mutated amino acids
>HC4
(SEQ ID NO: 129)
EVQLVESGGGLVQPGGSLRLSCAASGEDFSRYWMSWVRQAPGKGLEWVAEIHPYSSTINYTPSVKDRETISRDNA
KNSLYLQMNSLRSEDTAVYYCARAFAYWGQGTLVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPSNTKVDKKVEPKSCDKTHTCPPC
PAPELLGGPSVFLFPPKPKDTLMISRTPEVTCVVVDVSHEDPEVKENWYVDGVEVHNAKTKPREEQYNSTYRVVS
VLTVLHQDWLNGKEYKCKVSNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALHNHYTQKSLSLSPGK
>HC4 D270A (Kabat numbering)
(SEQ ID NO: 161)
EVQLVESGGGLVQPGGSLRLSCAASGFDFSRYWMSWVRQAPGKGLEWVAEIHPYSSTINYTPSVKDRFTISRDNA
KNSLYLQMNSLRSEDTAVYYCARAFAYWGQGTLVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPSNTKVDKKVEPKSCDKTHTCPPC
PAPELLGGPSVFLFPPKPKDTLMISRTPEVTCVVVDVSHEAPEVKENWYVDGVEVHNAKTKPREEQYNSTYRVVS
VLTVLHQDWLNGKEYKCKVSNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALHNHYTQKSLSLSPGK
>HC4 P329A (Kabat numbering)
(SEQ ID NO: 162)
EVQLVESGGGLVQPGGSLRLSCAASGEDFSRYWMSWVRQAPGKGLEWVAEIHPYSSTINYTPSVKDRFTISRDNA
KNSLYLQMNSLRSEDTAVYYCARAFAYWGQGTLVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPSNTKVDKKVEPKSCDKTHTCPPC
PAPELLGGPSVFLFPPKPKDTLMISRTPEVTCVVVDVSHEDPEVKENWYVDGVEVHNAKTKPREEQYNSTYRVVS
VLTVLHQDWLNGKEYKCKVSNKALAAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALHNHYTQKSLSLSPGK
>HC4 P331S (Kabat numbering)
(SEQ ID NO: 163)
EVQLVESGGGLVQPGGSLRLSCAASGFDFSRYWMSWVRQAPGKGLEWVAEIHPYSSTINYTPSVKDRFTISRDNA
KNSLYLQMNSLRSEDTAVYYCARAFAYWGQGTLVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPSNTKVDKKVEPKSCDKTHTCPPC
PAPELLGGPSVFLFPPKPKDTLMISRTPEVTCVVVDVSHEDPEVKFNWYVDGVEVHNAKTKPREEQYNSTYRVVS
VLTVLHQDWINGKEYKCKVSNKALPASIEKTISKAKGQPREPQVYTLPPSRDELTKNQVSLTCLVKGFYPSDIAV
EWESNGQPENNYKTTPPVLDSDGSFFLYSKLTVDKSRWQQGNVESCSVMHEALHNHYTQKSLSLSPGK
>Mutated Fc with D270A (Kabat numbering)
(SEQ ID NO: 164)
ASTKGPSVFPLAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVFLFPPKPKDTLMISRTPEVTCVVVDVS
HEAPEVKENWYVDGVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLYSKLTVDKSRW
QQGNVFSCSVMHEALHNHYTQKSLSLSPGK
>Mutated Fc with P329A (Kabat numbering)
(SEQ ID NO: 165)
ASTKGPSVFPLAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVELFPPKPKDTLMISRTPEVTCVVVDVS
HEDPEVKENWYVDGVEVHNAKTKPREEQYNSTYRVVSVLTVLHQDWLNGKEYKCKVSNKALAAPIEKTISKAKGQ
PREPQVYTLPPSRDELTKNQVSLTCLVKGFYPSDIAVEWESNGQPENNYKTTPPVLDSDGSFFLYSKLTVDKSRW
QQGNVFSCSVMHEALHNHYTQKSLSLSPGK
>Mutated Fc with P331S (Kabat numbering)
(SEQ ID NO: 166)
ASTKGPSVFPLAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVFLFPPKPKDTLMISRTPEVTCVVVDVS
HEDPEVKENWYVDGVEVHNAKTKPREEQYNSTYRVVSVLTVLHQDWINGKEYKCKVSNKALPASIEKTISKAKGQ
PREPQVYTIPPSRDELTKNQVSLTCLVKGFYPSDIAVEWESNGQPENNYKTTPPVLDSDGSFFLYSKLTVDKSRW
QQGNVFSCSVMHEALHNHYTQKSLSLSPGK

1. An antibody or antigen-binding fragment thereof, the antibody or antibody fragment comprising at least one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13.

2. The antibody or antigen-binding fragment thereof according to claim 1, wherein the antibody or antigen-binding fragment thereof is:
an intact antibody or an antigen-binding fragment thereof.

3-6. (canceled)

7. The antibody or antigen-binding fragment thereof according to claim 1, wherein the antibody or antigen-binding fragment thereof comprises or consists of the VH4-VL2 sequences and/or the VH5-VL2 sequences.

8. The antibody or antigen-binding fragment thereof according to claim 1, wherein the antibody or antigen-binding fragment thereof:
has specificity for a splice variant of vascular endothelial growth factor (VEGF), wherein the splice variant is VEGF-A₁₆₅b or any VEGF sequence containing the sequence encoded by Exon 8b of the VEGF gene (such as VEGF-Ax and VEGF₁₈₉b); and
does not have specificity for other VEGF splice variants not containing exon 8b, such as VEGF-A₁₆₅a.

9. The antibody or antigen-binding fragment thereof according to claim 1, further comprising a moiety that increases the in vivo half-life of the antibody or antigen-binding fragment thereof.

10. The antibody or antigen-binding fragment thereof according to claim 1, wherein the antibody or antigen-binding fragment thereof is PEGylated.

11. The antibody or antigen-binding fragment thereof according to claim 1, further comprising a cytotoxic moiety.

12. The antibody or antigen-binding fragment thereof according to claim 1, further comprising a detectable moiety.

13. A pharmaceutical composition comprising an effective amount of the antibody or antigen-binding fragment thereof according to claim 1, and a pharmaceutically-acceptable diluent, carrier or excipient.

14. The pharmaceutical composition according to claim 13 adapted for subcutaneous, intravenous, intramuscular, intracranial, or intraocular delivery.

15. A kit comprising the antibody or antigen-binding fragment thereof according to claim 1.

16. A medicament comprising:
the antibody or antigen-binding fragment thereof according to claim 1.

17. (canceled)

18. (canceled)

19. A method of treatment or diagnosis of a disease, syndrome or condition, the method comprising administering an effective amount of the antibody or antigen-binding fragment thereof according to claim 1;
wherein the disease, syndrome, or condition comprises a VEGF-associated diseases; Ischemia; Peripheral arterial disease (PAD); Atherosclerosis; a condition associated with diabetes; Sclerosis; Raynaud's syndrome; Arthritis; an ischemia-associated cutaneous condition; a retinal ischemic disease; a lung condition associated with altered angiogenesis; Inflammatory bowel disease; a neuronal ischemic condition; Stroke; Pre-eclampsia; Hypertension; Obesity; Hair loss; Kidney failure; Angiogenesis/revascularisation; Cancer; Deep vein thrombosis (DVT); Refractory angina; and Myocardial infarction (MI), or a post-MI condition.

20. An in vitro/ex vivo method of diagnosis, wherein the method comprises subjecting an isolated sample to the antibody or antigen-binding fragment thereof according to claim 1.

21-25. (canceled)

26. The antibody or antigen-binding fragment thereof according to claim 1, wherein the antibody or antigen-binding fragment thereof is recombinant.

27. The antibody or antigen-binding fragment thereof according to claim 1, wherein the antibody or antigen-binding fragment thereof is murine, chimeric, human or humanised.

28. The antibody or antigen-binding fragment thereof of claim 9, wherein the moiety comprises a post translational modification comprising polyethylene glycol (PEG), glycosylation, a fatty acid, dextran, or a genetic fusion protein.

29. The antibody or antigen-binding fragment thereof of claim 28, wherein the genetic fusion protein is human serum albumin or a cytokine.

30. The antibody or antigen-binding fragment thereof of claim 11, wherein the cytotoxic moiety comprises at least one of:
a radioisotope;
a toxin; or
a chemotherapeutic agent.

31. The antibody or antigen-binding fragment thereof of claim 12, wherein the detectable moiety comprises one or more of:
a radioisotope; or
a paramagnetic isotope.


SEQUENCE LISTING (166 sequences)

SEQ ID NO: 1 - protein, 8 aa, synthetic construct
GFDFSRYW

SEQ ID NO: 2 - protein, 8 aa, synthetic construct
IHPYSSTI

SEQ ID NO: 3 - protein, 6 aa, synthetic construct
ARAFAY

SEQ ID NO: 4 - protein, 11 aa, synthetic construct
QSLLDSDGKTY

SEQ ID NO: 5 - skipped

SEQ ID NO: 6 - protein, 9 aa, synthetic construct
WQGTHFPYT

SEQ ID NO: 7 - protein, 6 aa, synthetic construct
RYWMSW

SEQ ID NO: 8 - protein, 17 aa, synthetic construct
EIHPYSSTINYTPSVKD

SEQ ID NO: 9 - protein, 4 aa, synthetic construct
AFAY

SEQ ID NO: 10 - protein, 16 aa, synthetic construct
RSSQSLLDSDGKTYLN

SEQ ID NO: 11 - protein, 16 aa, synthetic construct
KSSQSLLDSDGKTYLN

SEQ ID NO: 12 - protein, 7 aa, synthetic construct
LVSKLDS

SEQ ID NO: 13 - protein, 9 aa, synthetic construct
WQGTHFPYT

SEQ ID NO: 14 - protein, 16 aa, synthetic construct
  VARIANT 1: note = Wherein X is R or K
XSSQSLLDSDGKTYLN

SEQ ID NO: 15 - protein, 113 aa, synthetic construct
EVKLLESGGGLVQPGGSLKLSCAASGFDFSRYWMSWVRQAPGKGLEWIGEIHPYSSTINYTPSVKDKFIISRDNAKNTLYLQMSEVRSEDTALYYCARAFAYWGQGTLVTVSA

SEQ ID NO: 16 - protein, 113 aa, synthetic construct
EVQLLESGGGLVKPGGSLRLSCAASGFDFSRYWMSWIRQAPGKGLEWVSEIHPYSSTINYTPSVKDRFTISRDNAKNSLYLQMNSLRAEDTAVYYCARAFAYWGQGTLVTVSS

SEQ ID NO: 17 - protein, 113 aa, synthetic construct
EVQLVESGGGLVQPGGSLRLSCAASGFDFSRYWMSWVRQAPGKGLEWVAEIHPYSSTINYTPSVKDRFIISRDNAKNSVYLQLNSLRAEDTAVYYCARAFAYWGQGTLVTVSS

SEQ ID NO: 18 - protein, 113 aa, synthetic construct
EVQLVESGGGLVQPGGSLRLSCAASGFDFSRYWMSWVRQAPGKGLEWISEIHPYSSTINYTPSVKDRFTISRDNAKNSLYLQMNSLRDEDTALYYCARAFAYWGQGTLVTVSS

SEQ ID NO: 19 - protein, 113 aa, synthetic construct
EVQLVESGGGLVQPGGSLRLSCAASGFDFSRYWMSWVRQAPGKGLEWVAEIHPYSSTINYTPSVKDRFTISRDNAKNSLYLQMNSLRSEDTAVYYCARAFAYWGQGTLVTVSS

SEQ ID NO: 20 - protein, 113 aa, synthetic construct
EVQLVESGGGLVQPGGSLRLSCAASGFDFSRYWMSWVRQAPGKGLEWVSEIHPYSSTINYTPSVKDRFTISRDNAKNSLYLQMNSLRAEDTAVYYCARAFAYWGQGTLVTVSA

SEQ ID NO: 21 - protein, 113 aa, synthetic construct
  VARIANT 34: note = Wherein X is M or R
  VARIANT 44: note = Wherein X is G or E
  VARIANT 68: note = Wherein X is F or L
  VARIANT 74: note = Wherein X is N or S
  VARIANT 18: note = Wherein X is L or P
EVKLLESGGGLVQPGGSXKLSCAASGFDFSRYWXSWVRQAPGKXLEWIGEIHPYSSTINYTPSVKDKXIISRDXAKNTLYLQMSEVRSEDTALYYCARAFAYWGQGTLVTVSA

SEQ ID NO: 22 - protein, 146 aa, synthetic construct
EVKLLESGGGLVQPGGSLKLSCAASGFDFSRYWMSWVRQAPGKGLEWIGEIHPYSSTINYTPSVKDKFIISRDNAKNTLYLQMSEVRSEDTALYYCARAFAYWGQGTLVTVSAAKTTPPSVYPLAPGSAAQTNSMVTLGCLVKGYF

SEQ ID NO: 23 - protein, 113 aa, synthetic construct
DIVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPYTFGGGTKVEIKR

SEQ ID NO: 24 - protein, 113 aa, synthetic construct
DVVMTQSPLSLPVTLGQPASISCKSSQSLLDSDGKTYLNWFQQRPGQSPRRLIYLVSKLDSGVPDRFSGSGSGTDFTLKISRVEAEDVGVYYCWQGTHFPYTFGGGTKVEIKR

SEQ ID NO: 25 - protein, 113 aa, synthetic construct
DVVMTQSPLSLPVTLGQPASISCRSSQSLLDSDGKTYLNWELQRPGQSPRRLIYLVSKLDSGVPDRFSGSGSGTDFTLKISRVEAEDVGVYYCWQGTHFPYTFGQGTKVEIKR

SEQ ID NO: 26 - protein, 113 aa, synthetic construct
DIVMTQTPLSLSVTPGQPASISCKSSQSLLDSDGKTYLNWYLQKPGQSPQLLIYLVSKLDSGVPDRFSGSGSGTDFTLKISRVEPEDVGVYYCWQGTHFPYTFGGGTKVEVKR

SEQ ID NO: 27 - protein, 113 aa, synthetic construct
DIVMTQTPLSSPVTLGQPASISCRSSQSLLDSDGKTYLNWLQQRPGQPPRLLIYLVSKLDSGVPDRFSGSGAGTDFTLKISRVEAEDVGVYYCWQGTHFPYTFGQGTKLEIKR

SEQ ID NO: 28 - protein, 113 aa, synthetic construct
DIVMTQTPLSLSVTPGQPASISCKSSQSLLDSDGKTYLNWYLQKPGQSPQLLIYLVSKLDSGVPDRFSGSGSGTDFTLKISRVEAEDVGVYYCWQGTHFPYTFGGGTKVEIKR

SEQ ID NO: 29 - protein, 113 aa, synthetic construct
  VARIANT 7: note = Wherein X is T or S
  VARIANT 10: note = Wherein X is T or S
  VARIANT 11: note = Wherein X is L or S
  VARIANT 12: note = Wherien X is S or P
  VARIANT 15: note = Wherein X is I, L, or P
  VARIANT 24: note = Wherein X is K or R
  VARIANT 41: note = Wherein X is L, F, E, or Y
  VARIANT 42: note = Wherein X is L or Q
  VARIANT 44: note = Wherein X is R or K
  VARIANT 48: note = Wherein X is S or P
  VARIANT 50: note = Wherein X is K, R, or Q
  VARIANT 51: note = Wherein X is R or L
  VARIANT 68: note = Wherein X is T or S
  VARIANT 72: note = Wherein X is S or A
  VARIANT 85: note = Wherein X is A or P
  VARIANT 87: note = Wherein X is L or V
  VARIANT 105: note = Wherein X is G or Q
  VARIANT 109: note = Wherein X is V or L
  VARIANT 111: note = Wherein X is I or V
  VARIANT 2: note = Wherein X is I or V
DXVMTQXPLXXXVTXGQPASISCXSSQSLLDSDGKTYLNWXXQXPGQXPXXLIYLVSKLDSGVPDRFXGSGXGTDFTLKISRVEXEDXGVYYCWQGTHFPYTFGXGTKXEXKR

SEQ ID NO: 30 - protein, 147 aa, synthetic construct
DIVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPYTFGGGTKVEIKRADAAPTVSIFPPSSEQLTSGGASVVCFLNNFYPR

SEQ ID NO: 31 - protein, 130 aa, synthetic construct
DIVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPYTFGGGTKVEIKRADAAPTVSIFPPSSEQL

SEQ ID NO: 32 - protein, 19 aa, synthetic construct
MGWTLVFLFLLSVTAGVHS

SEQ ID NO: 33 - protein, 226 aa, synthetic construct
EVQLVESGGGLVQPGGSLRLSCAASGFDFSRYWMSWVRQAPGKGLEWVAEIHPYSSTINYTPSVKDRFTISRDNAKNSLYLQMNSLRSEDTAVYYCARAFAYWGQGTLVTVSSDVVMTQSPLSLPVTLGQPASISCRSSQSLLDSDGKTYLNWELQRPGQSPRRLIYLVSKLDSGVPDRFSGSGSGTDFTLKISRVEAEDVGVYYCWQGTHFPYTFGQGTKVEIKR

SEQ ID NO: 34 - protein, 226 aa, synthetic construct
EVQLVESGGGLVQPGGSLRLSCAASGFDFSRYWMSWVRQAPGKGLEWVSEIHPYSSTINYTPSVKDRFTISRDNAKNSLYLQMNSLRAEDTAVYYCARAFAYWGQGTLVTVSADVVMTQSPLSLPVTLGQPASISCRSSQSLLDSDGKTYLNWELQRPGQSPRRLIYLVSKLDSGVPDRFSGSGSGTDFTLKISRVEAEDVGVYYCWQGTHFPYTFGQGTKVEIKR

SEQ ID NO: 35 - protein, 330 aa, synthetic construct [sequence omitted]

SEQ ID NO: 36 - protein, 106 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 9 aa, synthetic construct
TCRSLTRKD

SEQ ID NO: 38 - protein, 123 aa, synthetic construct
EVKLLESGGGLVQPGGSLKLSCAASGFDFSRYWMSWVRQAPGKGLEWIGEIHPYSSTINYTPSVKDKFIISRDNAKNTLYLQMSEVRSEDTALYYCARAFAYWGQGTLVTVSAAKTTPPPVYP

SEQ ID NO: 39 - DNA, 369 bp, synthetic construct
  source 1..369: mol_type = other DNA
gaggtgaagcttctcgaatctggaggtggcctggtgcagcctggaggatccctgaaactctcctgtgcagcctcaggattcgattttagtagatactggatgagttgggtccggcaggctccagggaaagggctagaatggattggagaaattcatccatatagcagtacgataaactatacgccatctgtaaaggataaattcatcatctccagagacaacgccaaaaatacgctgtacctgcaaatgagcgaagtgagatctgaggacacagccctttattactgtgcaagggcgtttgcttactggggccaagggactctggtcactgtctctgcagccaaaacgacacccccacccgtttatcct

SEQ ID NO: 40 - protein, 123 aa, synthetic construct
EVKLLESGGGLVQPGGSLKLSCAASGFDFSRYWMSWVRQAPGKGLEWIGEIHPYSSTINYTPSVKDKFIISRDNAKNTLYLQMSEVRSEDTALYYCARAFAYWGQGTLVTVSAAKTTPPSVYP

SEQ ID NO: 41 - DNA, 369 bp, synthetic construct
  source 1..369: mol_type = other DNA
gaggtgaagcttctcgaatctggaggtggcctggtgcagcctggaggatccctgaaactctcctgtgcagcctcaggattcgattttagtagatactggatgagttgggtccggcaggctccagggaaagggctagaatggattggagaaattcatccatatagcagtacgataaactatacgccatctgtaaaggataaattcatcatctccagagacaacgccaaaaatacgctgtacctgcaaatgagcgaagtgagatctgaggacacagccctttattactgtgcaagggcgtttgcttactggggccaagggactctggtcactgtctctgcagccaaaacgacacccccatccgtctatccc

SEQ ID NO: 42 - protein, 125 aa, synthetic construct
EVKLLESGGGLVQPGGSLKLSCAASGFDFSRYWRSWVRQAPGKGLEWIGEIHPYSSTINYTPSVKDKFIISRDNAKNTLYLQMSEVRSEDTALYYCARAFAYWGQGTLVTVSAAKTTPPSVYPLA

SEQ ID NO: 43 - DNA, 375 bp, synthetic construct
  source 1..375: mol_type = other DNA
gaggtgaagcttctcgaatctggaggtggcctggtgcagcctggaggatccctgaaactctcctgtgcagcctcaggattcgattttagtagatactggaggagttgggtccggcaggctccagggaaagggctagaatggattggagaaattcatccatatagtagtacgataaactatacgccatctgtaaaggataaattcatcatctccagagacaacgccaaaaatacgctgtacctgcaaatgagcgaagtgagatctgaggacacagccctttattactgtgcaagggcgtttgcttactggggccaagggactctggtcactgtctctgcagccaaaacgacacccccatccgtctatcccttggcc

SEQ ID NO: 44 - protein, 146 aa, synthetic construct
EVKLLESGGGLVQPGGSLKLSCAASGFDFSRYWMSWVRQAPGKGLEWIGEIHPYSSTINYTPSVKDKLIISRDNAKNTLYLQMSEVRSEDTALYYCARAFAYWGQGTLVTVSAAKTTPPSVYPLAPGSAAQTNSMVTLGCLVKGYF

SEQ ID NO: 45 - DNA, 438 bp, synthetic construct
  source 1..438: mol_type = other DNA
gaggtgaagcttctcgaatctggaggtggtctggtgcagcctggaggatccctgaaactctcctgtgcagcctcaggattcgattttagtagatactggatgagttgggtccggcaggctccagggaaagggctagaatggattggagaaattcatccatatagcagtacgataaactatacgccatctgtaaaggataaattaatcatctccagagacaacgccaaaaatacgctgtacctgcaaatgagcgaagtgagatctgaggacacagccctttattactgtgcaagggcgtttgcttactggggccaagggactctggtcactgtctctgcagccaaaacgacacccccatctgtctatccactggcccctggatctgctgcccaaactaactccatggtgaccctgggatgccttgtcaaaggttatttc

SEQ ID NO: 46 - protein, 125 aa, synthetic construct
EVKLLESGGGLVQPGGSLKLSCAASGFDFSRYWMSWVRQAPGKELEWIGEIHPYSSTINYTPSVKDKFIISRDNAKNTLYLQMSEVRSEDTALYYCARAFAYWGQGTLVTVSAAKTTPPPVYPLA

SEQ ID NO: 47 - DNA, 375 bp, synthetic construct
  source 1..375: mol_type = other DNA
gaggtgaagcttctcgaatctggaggtggcctggtgcagcctggaggatccctgaaactctcctgtgcagcctcaggattcgattttagtagatactggatgagttgggtccggcaggctccagggaaagagctagaatggattggagaaattcatccatatagcagtacgataaactatacgccatctgtaaaggataaattcatcatctccagagacaacgccaaaaatacgctgtacctgcaaatgagcgaagtgagatctgaggacacagccctttattactgtgcaagggcgtttgcttactggggccaagggactctggtcactgtctctgcagccaaaacgacacccccacccgtttatccattggcc

SEQ ID NO: 48 - protein, 146 aa, synthetic construct; identical to SEQ ID NO: 22

SEQ ID NO: 49 - DNA, 438 bp, synthetic construct
  source 1..438: mol_type = other DNA
gaggtgaagcttctcgaatctggaggtggcctggtgcagcctggaggatccctgaaactctcctgtgcagcctcaggattcgattttagtagatactggatgagttgggtccggcaggctccagggaaagggctagaatggattggagaaattcatccatatagcagtacgataaactatacgccatccgtaaaggataaattcatcatctccagagacaacgccaaaaatacgctgtacctgcaaatgagcgaagtgagatctgaggacacagccctttattactgtgcaagggcgtttgcttactggggccaagggactctggtcactgtctctgcagccaaaacgacacccccatctgtctatccactggcccctggatctgctgcccaaactaactccatggtgaccctgggatgcctggtcaaaggttatttc

SEQ ID NO: 50 - DNA, 438 bp, synthetic construct
  source 1..438: mol_type = other DNA
gaggtgaagcttctcgaatctggaggtggcctggtgcagcctggaggatccctgaaactctcctgtgcagcctcaggattcgattttagtagatactggatgagttgggtccggcaggctccagggaaagggctagaatggattggagaaattcatccatatagcagtacgataaactatacgccatctgtaaaggataaattcatcatctccagagacaacgccaaaaatacgctgtacctgcaaatgagcgaagtgagatctgaggacacagccctttattactgtgcaagggcgtttgcttactggggccaagggactctggtcactgtctctgcagccaaaacgacacccccatctgtctatccactggcccctggatctgctgcccaaactaactccatggtgaccctgggatgccttgtcaaaggttatttc

SEQ ID NO: 51 - protein, 146 aa, synthetic construct
EVKLLESGGGLVQPGGSPKLSCAASGFDFSRYWMSWVRQAPGKGLEWIGEIHPYSSTINYTPSVKDKFIISRDNAKNTLYLQMSEVRSEDTALYYCARAFAYWGQGTLVTVSAAKTTPPSVYPLAPGSAAQTNSMVTLGCLVKGYF

SEQ ID NO: 52 - DNA, 438 bp, synthetic construct
  source 1..438: mol_type = other DNA
gaggtgaagcttctcgaatctggaggtggcctggtgcagcctggaggatccccgaaactctcctgtgcagcctcaggattcgattttagtagatactggatgagttgggtccggcaggctccagggaaagggctagaatggattggagaaattcatccatatagcagtacgataaactatacgccatctgtaaaggataaattcatcatctccagagacaacgccaaaaatacgctgtacctgcaaatgagcgaagtgagatctgaggacacagccctttattactgtgcaagggcgtttgcttactggggccaagggactctggtcactgtctctgcagccaaaacgacacccccatccgtctatccactggcccctggatctgctgcccaaactaactccatggtgaccctgggatgcctggtcaaaggttatttc

SEQ ID NO: 53 - protein, 125 aa, synthetic construct
EVKLLESGGGLVQPGGSLKLSCAASGFDFSRYWMSWVRQAPGKGLEWIGEIHPYSSTINYTPSVKDKFIISRDNAKNTLYLQMSEVRSEDTALYYCARAFAYWGQGTLVTVSAAKTTPPPVYPLV

SEQ ID NO: 54 - DNA, 375 bp, synthetic construct
  source 1..375: mol_type = other DNA
gaggtgaagcttctcgaatctggaggtggcctggtgcagcctggaggatccctgaaactctcctgtgcagcctcaggattcgattttagtagatactggatgagttgggtccggcaggctccagggaaagggctagaatggattggagaaattcatccatatagcagtacgataaactatacgccatctgtaaaggataaattcatcatctccagagacaacgccaaaaatacgctgtacctgcaaatgagcgaagtgagatctgaggacacagccctttattactgtgcaagggcgtttgcttactggggccaagggactctggtcactgtctctgcagccaaaacgacacccccacccgtctatcccttggtc

SEQ ID NO: 55 - DNA, 438 bp, synthetic construct
  source 1..438: mol_type = other DNA
gaggtgaagcttctcgaatctggaggtggcctggtgcagcctggaggatccctgaaactctcctgtgcagcctcaggattcgattttagtagatactggatgagttgggtccggcaggctccagggaaagggctagaatggattggagaaattcatccatatagcagtacgataaactatacgccatctgtaaaggataaattcatcatctccagagacaacgccaaaaatacgctgtacctgcaaatgagcgaagtgagatctgaggacacagccctttattactgtgcaagggcgtttgcttactggggccaagggactctggtcactgtctctgcagccaaaacgacacccccatctgtctatccactggcccctggatctgctgcccaaactaactccatggtgaccctgggatgcctggtcaagggttatttc

SEQ ID NO: 56 - protein, 146 aa, synthetic construct
EVKLLESGGGLVQPGGSLKLSCAASGFDFSRYWMSWVRQAPGKGLEWIGEIHPYSSTINYTPSVKDKFIISRDSAKNTLYLQMSEVRSEDTALYYCARAFAYWGQGTLVTVSAAKTTPPSVYPLAPGSAAQTNSMVTLGCLVKGYF

SEQ ID NO: 57 - DNA, 438 bp, synthetic construct
  source 1..438: mol_type = other DNA
gaggtgaagcttctcgaatctggaggtggcctggtgcagcctggaggatccctgaaactctcctgtgcagcctcaggattcgattttagtagatactggatgagttgggtccggcaggctccagggaaagggctagaatggattggagaaattcatccatatagcagtacgataaactatacgccatctgtaaaggataaattcatcatctccagagacagcgccaaaaatacgctgtacctgcaaatgagcgaagtgagatctgaggacacagccctttattactgtgcaagggcgtttgcttactggggccaagggactctggtcactgtctctgcagccaaaacgacacccccatctgtctatccactggcccctggatctgctgcccaaactaactccatggtgaccctgggatgcctggtcaagggctatttc

SEQ ID NO: 58 - protein, 146 aa, synthetic construct
  VARIANT 18: note = Wherein X is L or P
  VARIANT 34: note = Wherein X is M or R
  VARIANT 44: note = Wherein X is G or E
  VARIANT 68: note = Wherein X is F or L
  VARIANT 74: note = Wherein X is N or S
  VARIANT 120: note = Wherein X is S or P
  VARIANT 124: note = Wherein X is L or is deleted
  VARIANT 125: note = Wherein X is A, V, or is deleted
  VARIANT 126: note = Wherein X is P or is deleted
  VARIANT 127: note = Wherien X is G or is deleted
  VARIANT 128: note = Wherein X is S or is deleted
  VARIANT 129: note = Wherien X is A or is deleted
  VARIANT 130: note = Wherein X is A or is deleted
  VARIANT 131: note = Wherein X is Q or is deleted
  VARIANT 132: note = Wherein X is T or is deleted
  VARIANT 133: note = Wherein X is N or is deleted
  VARIANT 134: note = Wherein X is S or is deleted
  VARIANT 135: note = Wherein X is M or is deleted
  VARIANT 136: note = Wherein X is V or is deleted
  VARIANT 137: note = Wherein X is T or is deleted
  VARIANT 138: note = Wherein X is L or is deleted
  VARIANT 139: note = Wherein X is G or is deleted
  VARIANT 140: note = Wherein X is C or is deleted
  VARIANT 141: note = Wherein X is L or is deleted
  VARIANT 142: note = Wherein X is V or is deleted
  VARIANT 143: note = Wherein X is K or is deleted
  VARIANT 144: note = Wherein X is G or is deleted
  VARIANT 145: note = Wherein X is Y or is deleted
  VARIANT 146: note = Wherein X is F or is deleted
EVKLLESGGGLVQPGGSXKLSCAASGFDFSRYWXSWVRQAPGKXLEWIGEIHPYSSTINYTPSVKDKXIISRDXAKNTLYLQMSEVRSEDTALYYCARAFAYWGQGTLVTVSAAKTTPPXVYPXXXXXXXXXXXXXXXXXXXXXXX

SEQ ID NO: 59-70 - skipped (12 entries)

SEQ ID NO: 71 - protein, 462 aa, synthetic construct
MGWTLVFLFLLSVTAGVHSEVKLLESGGGLVQPGGSLKLSCAASGFDFSRYWMSWVRQAPGKGLEWIGEIHPYSSTINYTPSVKDKFIISRDNAKNTLYLQMSEVRSEDTALYYCARAFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 462 aa, synthetic construct
MGWTLVFLFLLSVTAGVHSEVQLLESGGGLVKPGGSLRLSCAASGFDFSRYWMSWIRQAPGKGLEWVSEIHPYSSTINYTPSVKDRFTISRDNAKNSLYLQMNSLRAEDTAVYYCARAFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 462 aa, synthetic construct
MGWTLVFLFLLSVTAGVHSEVQLVESGGGLVQPGGSLRLSCAASGFDFSRYWMSWVRQAPGKGLEWVAEIHPYSSTINYTPSVKDRFIISRDNAKNSVYLQLNSLRAEDTAVYYCARAFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 462 aa, synthetic construct
MGWTLVFLFLLSVTAGVHSEVQLVESGGGLVQPGGSLRLSCAASGFDFSRYWMSWVRQAPGKGLEWISEIHPYSSTINYTPSVKDRFTISRDNAKNSLYLQMNSLRDEDTALYYCARAFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 462 aa, synthetic construct
MGWTLVFLFLLSVTAGVHSEVQLVESGGGLVQPGGSLRLSCAASGFDFSRYWMSWVRQAPGKGLEWVAEIHPYSSTINYTPSVKDRFTISRDNAKNSLYLQMNSLRSEDTAVYYCARAFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 462 aa, synthetic construct
MGWTLVFLFLLSVTAGVHSEVQLVESGGGLVQPGGSLRLSCAASGFDFSRYWMSWVRQAPGKGLEWVSEIHPYSSTINYTPSVKDRFTISRDNAKNSLYLQMNSLRAEDTAVYYCARAFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 239 aa, synthetic construct
MVSSAQFLGLLLLCFQGTRCDIVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 239 aa, synthetic construct
MVSSAQFLGLLLLCFQGTRCDVVMTQSPLSLPVTLGQPASISCKSSQSLLDSDGKTYLNWFQQRPGQSPRRLIYLVSKLDSGVPDRFSGSGSGTDFTLKISRVEAEDVGVYYCWQGTHFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 239 aa, synthetic construct
MVSSAQFLGLLLLCFQGTRCDVVMTQSPLSLPVTLGQPASISCRSSQSLLDSDGKTYLNWFLQRPGQSPRRLIYLVSKLDSGVPDRFSGSGSGTDFTLKISRVEAEDVGVYYCWQGTHFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 239 aa, synthetic construct
MVSSAQFLGLLLLCFQGTRCDIVMTQTPLSLSVTPGQPASISCKSSQSLLDSDGKTYLNWYLQKPGQSPQLLIYLVSKLDSGVPDRFSGSGSGTDFTLKISRVEPEDVGVYYCWQGTHFPYTFGGGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 239 aa, synthetic construct
MVSSAQFLGLLLLCFQGTRCDIVMTQTPLSSPVTLGQPASISCRSSQSLLDSDGKTYLNWLQQRPGQPPRLLIYLVSKLDSGVPDRFSGSGAGTDFTLKISRVEAEDVGVYYCWQGTHFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 239 aa, synthetic construct
MVSSAQFLGLLLLCFQGTRCDIVMTQTPLSLSVTPGQPASISCKSSQSLLDSDGKTYLNWYLQKPGQSPQLLIYLVSKLDSGVPDRFSGSGSGTDFTLKISRVEAEDVGVYYCWQGTHFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 98 aa, synthetic construct
EVQLLESGGGLVKPGGSLRLSCAASGFDFSRYWMSWIRQAPGKGLEWVSEIHPYSSTINYTPSVKDRFTISRDNAKNSLYLQMNSLRAEDTAVYYCAR

SEQ ID NO: 84 - protein, 98 aa, synthetic construct
EVKLLESGGGLVQPGGSLKLSCAASGFDFSRYWMSWVRQAPGKGLEWIGEINPDSSTINYTPSLKDKFIISRDNAKNTLYLQMSKVRSEDTALYYCAR

SEQ ID NO: 85 - protein, 98 aa, synthetic construct
QVQLLESGGGLVKPGGSLRLSCAASGFTFSDYYMSWIRQAPGKGLEWVSYISSSSSYTNYADSVKGRFTISRDNAKNSLYLQMNSLRAEDTAVYYCAR

SEQ ID NO: 86 - protein, 100 aa, synthetic construct
DVVMTQSPLSLPVTLGQPASISCKSSQSLLDSDGKTYLNWFQQRPGQSPRRLIYLVSKLDSGVPDRFSGSGSGTDFTLKISRVEAEDVGVYYCWQGTHEP

SEQ ID NO: 87 - protein, 100 aa, synthetic construct
DVVMTQSPLSLPVTLGQPASISCRSSQSLVHSDGNTYLNWFQQRPGQSPRRLIYKVSNRDSGVPDRFSGSGSGTDFTLKISRVEAEDVGVYYCMQGTHWP

SEQ ID NO: 88 - protein, 132 aa, synthetic construct
MGWTLVFLFLLSVTAGVHSEVKLLESGGGLVQPGGSLKLSCAASGFDFSRYWMSWVRQAPGKGLEWIGEIHPYSSTINYTPSVKDKFIISRDNAKNTLYLQMSEVRSEDTALYYCARAFAYWGQGTLVTVSA

SEQ ID NO: 89 - protein, 132 aa, synthetic construct
MGWTLVFLFLLSVTAGVHSEVQLLESGGGLVKPGGSLRLSCAASGFDFSRYWMSWIRQAPGKGLEWVSEIHPYSSTINYTPSVKDRFTISRDNAKNSLYLQMNSLRAEDTAVYYCARAFAYWGQGTLVTVSS

SEQ ID NO: 90 - protein, 132 aa, synthetic construct
MGWTLVFLFLLSVTAGVHSEVQLVESGGGLVQPGGSLRLSCAASGFDFSRYWMSWVRQAPGKGLEWVAEIHPYSSTINYTPSVKDRFIISRDNAKNSVYLQLNSLRAEDTAVYYCARAFAYWGQGTLVTVSS

SEQ ID NO: 91 - protein, 132 aa, synthetic construct
MGWTLVFLFLLSVTAGVHSEVQLVESGGGLVQPGGSLRLSCAASGFDFSRYWMSWVRQAPGKGLEWISEIHPYSSTINYTPSVKDRFTISRDNAKNSLYLQMNSLRDEDTALYYCARAFAYWGQGTLVTVSS

SEQ ID NO: 92 - protein, 132 aa, synthetic construct
MGWTLVFLFLLSVTAGVHSEVQLVESGGGLVQPGGSLRLSCAASGFDFSRYWMSWVRQAPGKGLEWVAEIHPYSSTINYTPSVKDRFTISRDNAKNSLYLQMNSLRSEDTAVYYCARAFAYWGQGTLVTVSS

SEQ ID NO: 93 - protein, 132 aa, synthetic construct
MGWTLVFLFLLSVTAGVHSEVQLVESGGGLVQPGGSLRLSCAASGFDFSRYWMSWVRQAPGKGLEWVSEIHPYSSTINYTPSVKDRFTISRDNAKNSLYLQMNSLRAEDTAVYYCARAFAYWGQGTLVTVSA

SEQ ID NO: 94 - protein, 113 aa, synthetic construct
DVVMTQSPLSLPVTLGQPASISCKSSQSLLDSDGKTYLNWFQQRPGQSPRRLIYLVSKLDSGVPDRFSGSGSGTDFTLKISRVEAEDVGVYYCWQGTHEPYTFGGGTKVEIKR

SEQ ID NO: 95 - protein, 113 aa, synthetic construct
DVVMTQSPLSLPVTLGQPASISCRSSQSLLDSDGKTYLNWELQRPGQSPRRLIYLVSKLDSGVPDRFSGSGSGTDFTLKISRVEAEDVGVYYCWQGTHEPYTFGQGTKVEIKR

SEQ ID NO: 96 - protein, 113 aa, synthetic construct
DIVMTQTPLSLSVTPGQPASISCKSSQSLLDSDGKTYLNWYLQKPGQSPQLLIYLVSKLDSGVPDRFSGSGSGTDFTLKISRVEPEDVGVYYCWQGTHEPYTFGGGTKVEVKR

SEQ ID NO: 97 - protein, 113 aa, synthetic construct
DIVMTQTPLSSPVTLGQPASISCRSSQSLLDSDGKTYLNWLQQRPGQPPRLLIYLVSKLDSGVPDRFSGSGAGTDFTLKISRVEAEDVGVYYCWQGTHEPYTFGQGTKLEIKR

SEQ ID NO: 98 - protein, 113 aa, synthetic construct
DIVMTQTPLSLSVTPGQPASISCKSSQSLLDSDGKTYLNWYLQKPGQSPQLLIYLVSKLDSGVPDRFSGSGSGTDFTLKISRVEAEDVGVYYCWQGTHEPYTFGGGTKVEIKR

SEQ ID NO: 99 - protein, 443 aa, synthetic construct
EVKLLESGGGLVQPGGSLKLSCAASGFDFSRYWMSWVRQAPGKGLEWIGEIHPYSSTINYTPSVKDKFIISRDNAKNTLYLQMSEVRSEDTALYYCARAFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 443 aa, synthetic construct
EVKLLESGGGLVQPGGSLKLSCAASGFDFSRYWMSWVRQAPGKGLEWIGEIHPYSSTINYTPSVKDKFIISRDNAKNTLYLQMSEVRSEDTALYYCARAFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 443 aa, synthetic construct
EVKLLESGGGLVQPGGSLKLSCAASGFDFSRYWMSWVRQAPGKGLEWIGEIHPYSSTINYTPSVKDKFIISRDNAKNTLYLQMSEVRSEDTALYYCARAFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 443 aa, synthetic construct
EVQLVESGGGLVQPGGSLRLSCAASGFDFSRYWMSWVRQAPGKGLEWVAEIHPYSSTINYTPSVKDRFTISRDNAKNSLYLQMNSLRSEDTAVYYCARAFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 443 aa, synthetic construct
EVQLVESGGGLVQPGGSLRLSCAASGFDFSRYWMSWVRQAPGKGLEWVSEIHPYSSTINYTPSVKDRFTISRDNAKNSLYLQMNSLRAEDTAVYYCARAFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 443 aa, synthetic construct
EVQLVESGGGLVQPGGSLRLSCAASGFDFSRYWMSWVRQAPGKGLEWVAEIHPYSSTIQYTPSVKDRFTISRDNAKNSLYLQMNSLRSEDTAVYYCARAFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 443 aa, synthetic construct
EVQLVESGGGLVQPGGSLRLSCAASGFDFSRYWMSWVRQAPGKGLEWVSEIHPYSSTIQYTPSVKDRFTISRDNAKNSLYLQMNSLRAEDTAVYYCARAFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 443 aa, synthetic construct
EVQLVESGGGLVQPGGSLRLSCAASGFDFSRYWMSWVRQAPGKGLEWVAEIHPYSSTINYAPSVKDRFTISRDNAKNSLYLQMNSLRSEDTAVYYCARAFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 443 aa, synthetic construct
EVQLVESGGGLVQPGGSLRLSCAASGFDFSRYWMSWVRQAPGKGLEWVSEIHPYSSTINYAPSVKDRFTISRDNAKNSLYLQMNSLRAEDTAVYYCARAFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 219 aa, synthetic construct
DIVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 219 aa, synthetic construct
DVVMTQSPLSLPVTLGQPASISCKSSQSLLDSDGKTYLNWFQQRPGQSPRRLIYLVSKLDSGVPDRFSGSGSGTDFTLKISRVEAEDVGVYYCWQGTHFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 219 aa, synthetic construct
DVVMTQSPLSLPVTLGQPASISCRSSQSLLDSDGKTYLNWFLQRPGQSPRRLIYLVSKLDSGVPDRFSGSGSGTDFTLKISRVEAEDVGVYYCWQGTHFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 219 aa, synthetic construct
DVVMTQSPLSLPVTLGQPASISCKSSQSLLDADGKTYLNWFQQRPGQSPRRLIYLVSKLDSGVPDRFSGSGSGTDFTLKISRVEAEDVGVYYCWQGTHFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 219 aa, synthetic construct
DVVMTQSPLSLPVTLGQPASISCRSSQSLLDADGKTYLNWFLQRPGQSPRRLIYLVSKLDSGVPDRFSGSGSGTDFTLKISRVEAEDVGVYYCWQGTHFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 219 aa, synthetic construct
DVVMTQSPLSLPVTLGQPASISCKSSQSLLDSEGKTYLNWFQQRPGQSPRRLIYLVSKLDSGVPDRFSGSGSGTDFTLKISRVEAEDVGVYYCWQGTHFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 219 aa, synthetic construct
DVVMTQSPLSLPVTLGQPASISCRSSQSLLDSEGKTYLNWFLQRPGQSPRRLIYLVSKLDSGVPDRFSGSGSGTDFTLKISRVEAEDVGVYYCWQGTHFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 219 aa, synthetic construct
DVVMTQSPLSLPVTLGQPASISCKSSQSLLDSDAKTYLNWFQQRPGQSPRRLIYLVSKLDSGVPDRFSGSGSGTDFTLKISRVEAEDVGVYYCWQGTHFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 219 aa, synthetic construct
DVVMTQSPLSLPVTLGQPASISCRSSQSLLDSDAKTYLNWFLQRPGQSPRRLIYLVSKLDSGVPDRFSGSGSGTDFTLKISRVEAEDVGVYYCWQGTHFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 219 aa, synthetic construct
DVVMTQSPLSLPVTLGQPASISCKSSQSLLDSDGKTYLNWFQQRPGQSPRRLIYLVSKLESGVPDRFSGSGSGTDFTLKISRVEAEDVGVYYCWQGTHFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 219 aa, synthetic construct
DVVMTQSPLSLPVTLGQPASISCRSSQSLLDSDGKTYLNWFLQRPGQSPRRLIYLVSKLESGVPDRFSGSGSGTDFTLKISRVEAEDVGVYYCWQGTHFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 128 aa, synthetic construct
  UNSURE 31..34: note = Wherein X is unknown
  UNSURE 60..61: note = Wherein X is unknown
  UNSURE 73: note = Wherein X is unknown
  UNSURE 108..114: note = Wherein X is unknown
  UNSURE 10: note = Wherein X is unknown
EVKLLESGGXGLVQPGGSLKLSCAASGFDFXXXXSRYWMSWVRQAPGKGLEWIGEIHPYXXSSTINYTPSVKXDKFIISRDNAKNTLYLQMSEVRSEDTALYYCARAXXXXXXXFAYWGQGTLVTVSA

SEQ ID NO: 120 - protein, 9 aa, synthetic construct
GPSNDQEKR

SEQ ID NO: 121 - protein, 4 aa, synthetic construct
LALA

SEQ ID NO: 122 - protein, 98 aa, synthetic construct
  VARIANT 14: note = Wherein X is K or Q
  VARIANT 19: note = Wherein X is R or K
  VARIANT 37: note = Wherein X is I or V
  VARIANT 48: note = Wherein X is V or I
  VARIANT 49: note = Wherein X is S or G
  VARIANT 52: note = Wherein X is H or N
  VARIANT 54: note = Wherein X is Y or D
  VARIANT 64: note = Wherein X is V or L
  VARIANT 67: note = Wherein X is R or K
  VARIANT 69: note = Wherein X is T or I
  VARIANT 78: note = Wherein X is S or T
  VARIANT 84: note = wherein X is N or S
  VARIANT 85: note = Wherein X is S or K
  VARIANT 86: note = Wherein X is L or V
  VARIANT 88: note = Wherein X is A or S
  VARIANT 93: note = Wherein X is V or L
  VARIANT 3: note = Wherein X is Q or K
EVXLLESGGGLVXPGGSLXLSCAASGFDFSRYWMSWXRQAPGKGLEWXXEIXPXSSTINYTPSXKDXFXISRDNAKNXLYLQMXXXRXEDTAXYYCAR

SEQ ID NO: 123 - protein, 98 aa, synthetic construct
  VARIANT 28: note = Wherein X is D or T
  VARIANT 31: note = Wherein X is R or D
  VARIANT 33: note = Wherein X is W or Y
  VARIANT 50: note = Wherein X is E or Y
  VARIANT 52: note = Wherien X is H or S
  VARIANT 53: note = Wherein X is P or S
  VARIANT 54: note = Wherein X is Y or S
  VARIANT 57: note = Wherein X is T or Y
  VARIANT 58: note = Wherein X is I or T
  VARIANT 61: note = Wherein X is T or A
  VARIANT 62: note = Wherein X is P or D
  VARIANT 66: note = Wherein X is D or G
  VARIANT 1: note = Wherein X is E or Q
XVQLLESGGGLVKPGGSLRLSCAASGFXFSXYXMSWIRQAPGKGLEWVSXIXXXSSXXNYXXSVKXRFTISRDNAKNSLYLQMNSLRAEDTAVYYCAR

SEQ ID NO: 124 - protein, 100 aa, synthetic construct
  VARIANT 30: note = Wherein X is L or V
  VARIANT 31: note = Wherein X is D or H
  VARIANT 35: note = Wherein X is K or N
  VARIANT 55: note = Wherein X is L or K
  VARIANT 58: note = Wherein X is K or N
  VARIANT 59: note = Wherein X is L or R
  VARIANT 94: note = Wherein X is W or M
  VARIANT 99: note = Wherein X is E or W
  VARIANT 24: note = Wherein X is K or R
DVVMTQSPLSLPVTLGQPASISCXSSQSLXXSDGXTYLNWFQQRPGQSPRRLIYXVSXXDSGVPDRFSGSGSGTDFTLKISRVEAEDVGVYYCXQGTHXP

SEQ ID NO: 125 - protein, 443 aa, synthetic construct; identical to SEQ ID NO: 99

SEQ ID NO: 126 - protein, 443 aa, synthetic construct
EVQLLESGGGLVKPGGSLRLSCAASGFDFSRYWMSWIRQAPGKGLEWVSEIHPYSSTINYTPSVKDRFTISRDNAKNSLYLQMNSLRAEDTAVYYCARAFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 443 aa, synthetic construct
EVQLVESGGGLVQPGGSLRLSCAASGFDFSRYWMSWVRQAPGKGLEWVAEIHPYSSTINYTPSVKDRFIISRDNAKNSVYLQLNSLRAEDTAVYYCARAFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 443 aa, synthetic construct
EVQLVESGGGLVQPGGSLRLSCAASGFDFSRYWMSWVRQAPGKGLEWISEIHPYSSTINYTPSVKDRFTISRDNAKNSLYLQMNSLRDEDTALYYCARAFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 443 aa, synthetic construct
EVQLVESGGGLVQPGGSLRLSCAASGFDFSRYWMSWVRQAPGKGLEWVAEIHPYSSTINYTPSVKDRFTISRDNAKNSLYLQMNSLRSEDTAVYYCARAFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 443 aa, synthetic construct
EVQLVESGGGLVQPGGSLRLSCAASGFDFSRYWMSWVRQAPGKGLEWVSEIHPYSSTINYTPSVKDRFTISRDNAKNSLYLQMNSLRAEDTAVYYCARAFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 219 aa, synthetic construct; identical to SEQ ID NO: 108

SEQ ID NO: 132 - protein, 219 aa, synthetic construct; identical to SEQ ID NO: 109

SEQ ID NO: 133 - protein, 219 aa, synthetic construct; identical to SEQ ID NO: 110

SEQ ID NO: 134 - protein, 219 aa, synthetic construct
DIVMTQTPLSLSVTPGQPASISCKSSQSLLDSDGKTYLNWYLQKPGQSPQLLIYLVSKLDSGVPDRFSGSGSGTDFTLKISRVEPEDVGVYYCWQGTHFPYTFGGGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 219 aa, synthetic construct
DIVMTQTPLSSPVTLGQPASISCRSSQSLLDSDGKTYLNWLQQRPGQPPRLLIYLVSKLDSGVPDRFSGSGAGTDFTLKISRVEAEDVGVYYCWQGTHFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 219 aa, synthetic construct
DIVMTQTPLSLSVTPGQPASISCKSSQSLLDSDGKTYLNWYLQKPGQSPQLLIYLVSKLDSGVPDRFSGSGSGTDFTLKISRVEAEDVGVYYCWQGTHFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 144 aa, synthetic construct
DIVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPYTFGGGTKVEIKRADAAPTVSIFPPSSEQLTSGGASVVCFLNNF

SEQ ID NO: 138 - protein, 139 aa, synthetic construct
DIVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPYTFGGGTKVEIKRADAAPTVSIFPPSSEQLTSGGASVVC

SEQ ID NO: 139 - protein, 147 aa, synthetic construct
DIVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPYTFGGGTKVEIKRADAAPTVSIFPPSSEQLTSGGASVVCFLNNFYPK

SEQ ID NO: 140 - protein, 147 aa, synthetic construct
DIVMTQTPLTLSVTIGQPASISCKSSQCLLDSDGKTYLNWLLQRPGQSPKRLIYLVSKLDSGVPDRFTGSGSGTDFTLKISRVEAEDLGVYYCWQGTHFPYTFGGGTKVEIKRADAAPTVSIFPPSSEQLTSGGASVVCFLNNFYPR

SEQ ID NO: 141 - protein, 147 aa, synthetic construct; identical to SEQ ID NO: 30

SEQ ID NO: 142 - protein, 147 aa, synthetic construct; identical to SEQ ID NO: 30

SEQ ID NO: 143 - protein, 147 aa, synthetic construct; identical to SEQ ID NO: 139

SEQ ID NO: 144 - protein, 147 aa, synthetic construct; identical to SEQ ID NO: 139

SEQ ID NO: 145 - protein, 147 aa, synthetic construct; identical to SEQ ID NO: 30

SEQ ID NO: 146 - protein, 147 aa, synthetic construct; identical to SEQ ID NO: 30

SEQ ID NO: 147 - protein, 147 aa, synthetic construct
  VARIANT 2: note = Wherein X is I or V
  VARIANT 7: note = Wherein X is T or S
  VARIANT 11: note = Wherein X is L or S
  VARIANT 10: note = Wherein X is T or S
  VARIANT 12: note = Wherein X is S or P
  VARIANT 15: note = Wherein X is I, L or P
  VARIANT 24: note = Wherein X is K or R
  VARIANT 41: note = Wherein X is L, F, E or Y
  VARIANT 42: note = Wherein X is L or Q
  VARIANT 44: note = Wherein X is R or K
  VARIANT 48: note = Wherein X is S or P
  VARIANT 50: note = Wherein X is K, R or Q
  VARIANT 51: note = Wherein X is R or L
  VARIANT 68: note = Wherein X is T or S
  VARIANT 72: note = Wherein X is S or A
  VARIANT 85: note = Wherein X is A or P
  VARIANT 88: note = Wherein X is L or V
  VARIANT 105: note = Wherein X is G or Q
  VARIANT 109: note = Wherein X is V or L
  VARIANT 111: note = Wherein X is I or V
DXVMTQXPLXXXVTXGQPASISCXSSQSLLDSDGKTYLNWXXQXPGQXPXXLIYLVSKLDSGVPDRFXGSGXGTDFTLKISRVEXEDXGVYYCWQGTHFPYTFGXGTKXEXKRADAAPTVSIFPPSSEQLTSGGASVVCFLNNFYPR

SEQ ID NO: 148 - protein, 147 aa, synthetic construct; identical to SEQ ID NO: 30

SEQ ID NO: 149 - DNA, 433 bp, synthetic construct
  source 1..433: mol_type = other DNA
gatattgtgatgacccagactccactcactttgtcggttaccattggacagccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccgtacacgttcggaggggggaccaaggtggaaataaaacgggctgatgctgcaccaactgtatccatcttcccaccatccagtgagcagttaacatctggaggtgcctcagtcgtgtgcttcttgaacaacttct

SEQ ID NO: 150 - DNA, 417 bp, synthetic construct
  source 1..417: mol_type = other DNA
gatattgtgatgacccagactccactcactttgtcggttaccattggacagccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccgtacacgttcggaggggggaccaaggtggaaataaaacgggctgatgctgcaccaactgtatccatcttcccaccatccagtgagcagttaacatctggaggtgcctcagtcgtgtgc

SEQ ID NO: 151 - DNA, 441 bp, synthetic construct
  source 1..441: mol_type = other DNA
gatattgtgatgacccagactccactcactttgtcggttaccattggacagccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccgtacacgttcggaggggggaccaaggtggaaataaaacgggctgatgctgcaccaactgtatccatcttcccaccatccagtgagcagttaacatccggaggtgcctcagtcgtgtgcttcttgaacaacttctaccccaaa

SEQ ID NO: 152 - DNA, 441 bp, synthetic construct
  source 1..441: mol_type = other DNA
gatattgtgatgacccagactccactcactttgtcggttaccattggacagccagcctccatctcttgcaagtcaagtcagtgcctcttagatagtgatggaaagacatatttgaattggttgttacagaggccaggccagtctccaaagcgcctaatctatctggtgtctaaactggactctggagtccctgacaggttcactggcagtggatcagggacagatttcacgctgaaaatcagcagagtggaggctgaggatttgggagtttattattgctggcaaggtacacattttccgtacacgttcggaggggggaccaaggtggaaataaaacgggctgatgctgcaccaactgtatccatcttcccaccatccagtgagcagttaacatctggaggtgcctcagtcgtgtgcttcttgaacaacttctaccccaga

SEQ ID NO: 153 - DNA, 441 bp, synthetic construct
  source 1..441: mol_type = other DNA
gatattgtgatgacccagactccactcactttgtcggttaccattggacagccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccgtacacgttcggaggggggaccaaggtggaaataaaacgggctgatgctgcaccaactgtatccatcttcccaccatccagtgagcagttaacatctggaggtgcctcagtcgtgtgcttcttgaacaacttctaccccaga

SEQ ID NO: 154 - DNA, 441 bp, synthetic construct; identical to SEQ ID NO: 153
  source 1..441: mol_type = other DNA

SEQ ID NO: 155 - DNA, 441 bp, synthetic construct
  source 1..441: mol_type = other DNA
gatattgtgatgacccagactccactcactttgtcggttaccattggacagccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccgtacacgttcggaggggggaccaaggtggaaataaaacgggctgatgctgcaccaactgtatccatcttcccaccatccagtgagcagttaacatctggaggtgcctcagtcgtgtgcttcttgaacaacttctaccccaaa

SEQ ID NO: 156 - DNA, 441 bp, synthetic construct; identical to SEQ ID NO: 155
  source 1..441: mol_type = other DNA

SEQ ID NO: 157 - DNA, 441 bp, synthetic construct; identical to SEQ ID NO: 153
  source 1..441: mol_type = other DNA

SEQ ID NO: 158 - DNA, 441 bp, synthetic construct; identical to SEQ ID NO: 153
  source 1..441: mol_type = other DNA

SEQ ID NO: 159 - protein, 371 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRSLTRKD

SEQ ID NO: 160 - protein, 213 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRRSAGQEEGASLRVSGTRSLTRKD

SEQ ID NO: 161 - protein, 443 aa, synthetic construct
EVQLVESGGGLVQPGGSLRLSCAASGFDFSRYWMSWVRQAPGKGLEWVAEIHPYSSTINYTPSVKDRFTISRDNAKNSLYLQMNSLRSEDTAVYYCARAFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEAPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 443 aa, synthetic construct
EVQLVESGGGLVQPGGSLRLSCAASGFDFSRYWMSWVRQAPGKGLEWVAEIHPYSSTINYTPSVKDRFTISRDNAKNSLYLQMNSLRSEDTAVYYCARAFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 443 aa, synthetic construct
EVQLVESGGGLVQPGGSLRLSCAASGFDFSRYWMSWVRQAPGKGLEWVAEIHPYSSTINYTPSVKDRFTISRDNAKNSLYLQMNSLRSEDTAVYYCARAFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEAPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK